SEQUENCE LISTING (4 sequences)

SEQ ID NO: 1 - protein, 1646 aa, Artificial Sequence
  Synthetic Polypeptide
MAQILPIRFQEHLQLQNLGINPANIGFSTLTMESDKFICIREKVGEQAQVVIIDMNDPSNPIRRPISADSAIMNPASKVIFNIEMKSKMKAHTMTDDVTFWKWISLNTVALVTDNAVYHWSMEGESQPVKMFDRHSSLAGCQIINYRTDAALKAGKTLQIKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGNLSLLEHHHHHH

SEQ ID NO: 2 - protein, 256 aa, Artificial Sequence
  Synthetic Polypeptide
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRVADEAFYKQPFADVIGYVTNINHPCYSLEQAAEEAFVNDIDESSPGTEWERVARLCDFNPKSSKQAKDVSRMRSVLISLKQAPLVHLEHHHHHH

SEQ ID NO: 3 - protein, 1640 aa, Artificial Sequence
  Synthetic Polypeptide
MAQILPIRFQEHLQLQNLGINPANIGFSTLTMESDKFICIREKVGEQAQVVIIDMNDPSNPIRRPISADSAIMNPASKVIFNIEMKSKMKAHTMTDDVTFWKWISLNTVALVTDNAVYHWSMEGESQPVKMFDRHSSLAGCQIINYRTDAALKAGKTLQIKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGNLSLLE

SEQ ID NO: 4 - protein, 250 aa, Artificial Sequence
  Synthetic Polypeptide
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRVADEAFYKQPFADVIGYVTNINHPCYSLEQAAEEAFVNDIDESSPGTEWERVARLCDFNPKSSKQAKDVSRMRSVLISLKQAPLVHLE

(19) United States
(12) Patent Application Publication
(10) Pub. No.: US 2023/0065416 A1
(43) Pub. Date: Mar. 2, 2023

(54) CLATHRIN-CHIMETIC ANTIBODY RECEPTOR CONSTRUCTS FOR IMMUNE CELL ACTIVATION THERAPY IN VIVO

(71) Applicant: NANOTOMER, INC., Boston, MA (US)

(72) Inventors: David R. ELMALEH, Newton, MA (US); Reza ABDI, Newton, MA (US); Philip Ashton RICKARDT, Chestnut Hill, MA (US)

(73) Assignee: NANOTOMER, INC., Boston, MA (US)

(21) Appl. No.: 17/793,534

(22) PCT Filed: Jan. 26, 2021
(86) PCT No.: PCT/US2021/015058

§ 371 Date: Jul. 18, 2022

Related U.S. Application Data
(60) Provisional application No. 62/966,870, filed on Jan. 28, 2020.

(51) Int. Cl. C07K 14/725; C07K 16/18; A61P 35/00

(52) U.S. Cl. CPC C07K 14/7051; C07K 16/18; A61P 35/00; C07K 2319/03; C07K 2319/02

(57) ABSTRACT

The present invention relates to various clathrin constructs for immune cell activation in vivo. The present invention also relates to the use of various clathrin constructs for the detection or treatment of tumors, cancers, or other diseases or physiological condition. Disclosed herein are chimeric protein constructs including: a clathrin protein moiety; a chimeric antigen receptor (CAR) including: an ectodomain having an antigen binding domain; a transmembrane domain; and an endodomain having an intracellular signaling domain. Also disclosed herein are methods of producing a chimeric antigen receptor (CAR)-engineered cell of interest for in vivo activation of T-cells, B-cells and T-Reg cell, as well as others for inhibiting the growth, mutagenesis, or metastasis of a cancer, a tumor, or other neoplasm in a subject and methods of obtaining an image of a target cell of interest in a subject.

### CROSS-REFERENCE TO RELATED APPLICATION

This application claims priority to U.S. Provisional Patent Application 62/966,870, filed Jan. 28, 2020, which is incorporated by reference herein in its entirety.

### FIELD OF THE INVENTION

The present invention relates to various chimeric clathrin-chimeric antibody receptor (CAR) constructs for immune cell activation in vivo. The present invention also relates to the use of various chimeric clathrin-CAR constructs for the detection or treatment of cancers and other diseases.

### BACKGROUND OF THE INVENTION

The protein clathrin is important in the formation of coated vesicles. It serves as a molecular scaffold for vesicular uptake of cargo at the plasma membrane, where its assembly into cage-like or barrel-like lattices underlies the clathrin-coated pits of classical endocytosis. Clathrin-coated vesicles are found in all nucleated cells, from yeasts to humans.

Clathrin's triskelion shape, a three-legged object, is composed of three clathrin heavy chains and three light chains, which, when they interact, form a polyhedral lattice that surrounds the cellular vesicle and provides it with its rounded shape. The triskelia can form six-sided rings (hexagons) resulting in a flat lattice or five-sided rings (pentagons) resulting in a curved lattice formation) or combinations of the two. Alternatively, many triskelia can connect to form a basket-like structure (e.g., 36 triskelia), or a truncated icosahedron may be formed. Enclosure of a vesicle requires twelve pentagons in the lattice. One of the smallest clathrin cages (i.e., a mini-coat) comprises twelve pentagons and two hexagons. These vesicles are critical for secretory pathways, intracellular trafficking (including clathrin-mediated endocytosis [CME]) at the cell membrane, trans-Golgi network, and endosomal compartments, and for protection of the cytoplasm and other cellular components (e.g., degradative enzymes in lysosomes, oxidative enzymes in peroxisome, and cell-suicide activators in the intermembrane space of mitochondria). Clathrin-mediated endocytosis is a mechanism for endocytosis of activated cell surface receptors. After the vesicle buds into the cytoplasm, the clathrin coat disassembles and the clathrin triskelia are recycled. During mitosis, clathrin forms part of a complex that binds to the mitotic spindle and stabilizes the kinetochore fibers in order to crosslink microtubules. Membrane trafficking only occurs during interphase.

Each clathrin heavy chain (CHC) leg comprises several regions—a proximal region close to the central vertex and attached to a clathrin light chain (LC); a “knee” attaching the proximal region to a distal region; the distal region; an ankle; a linker; and an N-terminal region. Humans have two isoforms of clathrin heavy chain—CHC17, which is encoded by CLTC on chromosome 17, and CHC22, which is encoded by CLTD on chromosome 22—and two isoforms of clathrin light chain—LCa, which is encoded by CLTA on chromosome 9, and LCb, which is encoded by CLTB on chromosome 5. LCa and LCb associate with CHC17, but not with CHC22. Moreover, neuronal-specific splicing variants of LCa and LCb include 30-residue and 18-residue segments, respectively. Clathrin light chains comprise an N-terminal region; a conserved segment; an Hsc70 binding region (only in LCa); a calcium binding site; a CHC-binding region; optionally, a neuronal-specific segment; and a calmodulin-binding site. Generally, each leg comprises an approximately 190 kDa (1,676-residue) heavy chain and an approximately 25 kDa (approximately 200-220 residues) light chain. However, clathrin cages, barrels, baskets, and pits have a range of sizes and designs, and adaptor molecules assist with self-assembly and recruitment. In addition, clathrin accessory proteins may be transiently associated with coated vesicles comprising clathrin and its adaptor molecules, possibly to regulate the various steps.

While clathrin-coated vesicles are found in post-Golgi pathways and in endocytosis, coatomers coat protein I (COPI) and coat protein II (COPII) are found in many of the coats in the retrograde and anterograde pathways, respectively, between the endoplasmic reticulum (ER) and the Golgi.

The formation of clathrin-coated pits, and subsequently, vesicles, during clathrin-dependent endocytosis involves interactions of multifunctional adaptor proteins not only with clathrin and the plasma membrane, but also with several accessory proteins and phosphoinositides. The vesicles have a three-layered structure comprising an outer layer formed by a clathrin lattice, an internal layer having a lipid membrane with protein inclusions, and adaptor proteins in between the two layers, the adaptor proteins interacting with the lipid bilayer and the clathrin binding to the adaptors. One of the most abundant of the adaptor or accessory proteins is AP-2 (adaptor/assembly protein-2), which is targeted to the cell surface membrane, while AP-1 is involved in transport of proteins from the Golgi complex to early or late endosomes. AP-3 and AP-4 have also been identified. Other adaptor and accessory proteins include auxilin, Hsc70, epsins (Eps15, Eps15 interacting protein [epsin], CALM/AP180, huntingtin-interacting protein [HIP1], and HIP1R), ENTH/ANTH, disabled protein 2 (Dab2), ARH, Numb, TRIP8b, human stonin2 (hStn2), intersectin/Esc, dynamin, amphiphysin, and endophilin.

In recent years, immunotherapies, therapies that manipulate the patient's immune system to attack carcinogenic tumors, have been developed as an approach to cancer treatment. For example, monoclonal antibodies (mAbs) and bispecific monoclonal antibodies have been studied or used as oncology treatments for various cancers. However, target cells with low antigen expression may avoid recognition by mAbs or bispecific mAbs. Adoptive cell transfer (ACT) enhances cancer treatment by using the subject's immune cells to target and treat their cancer. ACT approaches include tumor-infiltrating lymphocytes (TILs), T-cells engineered to alter the specificity of the T-cell receptor (TCR), and chimeric antigen receptor (CAR) T-cell, (CAR) B-cells and (CAR) T regulatory cells (CAR Treg) therapy. The most widely developed ACT approach, chimeric antigen receptor (CAR) T-cell therapy, was approved for treating blood cancer in its advanced stages where it showed effective and accepted cancer treatment utility. More recently, in 2017, CAR T-cell therapy was expanded to and approved by the U.S. Food & Drug Administration (FDA) for the treatment of acute lymphoblastic leukemia (ALL) in children and later for the treatment of adults with advanced lymphomas, due to the successful treatment of cancers, autoimmune, inflammatory and neuroinflammatory disease, that otherwise had no cure.

CAR T-cell therapy can utilize T regulatory cells (Tregs), a subpopulation of T cells that can regulate ongoing immune reactions and play an important role in the control of autoimmunity, e.g., by secreting inhibitory cytokines, by interfering with the metabolism of T cells or other contacts, or by blocking T cell activation indirectly by interacting with antigen-presenting cells (APCs). Tregs may be polyclonal or antigen-specific (e.g., alloantigen-specific).

Researchers have been trying to expand these ACT treatments to several forms of cancer, such as melanoma, neuroblastoma, esophageal cancer, colorectal cancer, and breast cancer, which have solid tumors. CAR T-cell therapy has been shown to control tumor growth in xenograft models of T-cell leukemia and pancreatic cancer.

Typically, CAR T-cell, CAR B-cell or CAR Treg therapy involves removing blood from the patient in order to obtain the patient's T, B or Treg cells, inserting the chimeric antigen receptor (CAR) gene into the patient's T, B or Treg-cells in the laboratory to produce a CAR T-cell, CAR B-cells or CAR Treg cells (as T, B or Treg cells respectively with a specific chimeric antigen receptor), culturing and propagating the CAR T, B or Treg-cells, and infusing the CAR T, B or Treg-cells into the patient, where the antigens bind to cancer cells and kill them or where the antigens regulate inflammation (as with CAR-Treg). A CAR typically has an ectodomain outside the cell, a transmembrane domain, and an endodomain inside the cell.

However, CAR T, as an example, suffers from some disadvantages. It requires extracting the patient's immune T-cells, genetically engineering the T-cell to produce chimeric antigen receptors (CARs) to introduce antibody-like recognition, and expanding the modified T-cell, followed by lymphodepleting the patient by a chemotherapy regimen and subsequent infusion of the engineered CAR T-cells into the patient. The requirement to re-make the CAR-T product for each individual patient results in significant variation in cell product quality and in many cases prohibitively high costs of treatment. In addition, these manipulations are associated with significant toxicity and side effects that can be fatal. These side effects are due to massive toxin release, called cytokine release syndrome (CRS). Other side effects include B cell aplasia (large-scale B cell death), cerebral edema, and neurotoxicities, such as confusion and seizure-like activity. Finally, CAR-mediated recognition of cells with low antigen expression may pose a problem with respect to specificity, if the antigen in question is shared to some extent, e.g., by the corresponding non-diseased cells.

Further, this treatment is very expensive with costs amounting to hundreds of thousands of dollars per treatment. The increase of the population in the United States and many other countries and the concomitant increase in the percentage of the population suffering from various types of cancer, such as several types of aggressive lymphomas, melanoma, and pancreatic cancers, autoimmune or neuroinflammatory that have no efficacious treatment have contributed to the demand for new innate immune activation treatment of these fatal diseases.

Thus, there is a demand for, and it would be highly advantageous to have, alternative treatments that activate the adaptive immune system, which is less toxic and less expensive.

### SUMMARY OF DISCLOSURE

The present invention relates to various clathrin constructs with cancer therapy payloads (chemically derivatized, fused, and/or enclosed) for immune cell activation in vivo. The present invention also relates to the use of these various clathrin constructs for the detection or treatment, e.g., of cancers and immunoinflammatory diseases.

Unexpectedly, it has been found that a fusion of endogenous proteins (clathrin, light and/or heavy chains, either as peptides or as a self-assembled triskelion or clathrin cage, barrel, or basket) attached to chemotherapeutic payload or chimeric protein constructs (either as a fusion protein, as an attachment to the clathrin, or as attached to a triskelion or within or attached or embedded within a clathrin cage or basket, such as a self-assembled triskelion or cage) in several variant forms, resulting in new chemical chemotherapeutic entities for cancer and/or inflammatory treatments. Attachments can be made either to clathrin light chains and/or heavy chains separately, followed by assembly, or they can be made after the clathrin constructs have been assembled. According to another aspect, the present invention also provides chimeric construct entities for T and B cell, Treg immune cell activation therapy of, e.g., a specific cancer or an inflammatory or neuroinflammatory or autoimmune diseased or aberrant cellular entity, including, but not limited to, a cell infected by a virus or a bacterium or an infectious parasite or yeast. The present invention also provides synthetic biologically active constructs that activate the innate immune system in vivo for the treatment of cancer, infection, or other diseases following their administration, without removing and manipulating a patient's immune cells and readministering them.

In some aspects, provided herein are chimeric protein constructs comprising: a clathrin protein moiety or a functional derivative thereof; and a chimeric antigen receptor (CAR) comprising: an ectodomain comprising at least one antigen binding domain; a transmembrane domain; and an endodomain comprising an intracellular signaling domain.

In some aspects, provided herein are methods of producing a chimeric antigen receptor (CAR)-engineered cell of interest in vivo, the method comprising: providing a chimeric protein construct comprising: a clathrin protein moiety or a functional derivative thereof; and a chimeric antigen receptor (CAR) comprising: an ectodomain comprising at least one antigen binding domain; a transmembrane domain; and an endodomain comprising an intracellular signaling domain; administering the chimeric protein construct to a subject in need thereof; and transducing a target cell of interest in the subject with the chimeric protein construct to produce a CAR-engineered cell of interest.

In some aspects, provided herein are methods of inhibiting the growth, mutagenesis, or metastasis of a cancer, a tumor, or other neoplasm in a subject in need thereof, the method comprising: obtaining an antigenic profile of the cancer, the tumor, or the other neoplasm; providing an antigen binding domain in response to the antigenic profile of the cancer, the tumor, or the other neoplasm, wherein the antigen binding domain binds an antigen specific to the tumor or other neoplasm or an antigen specific to a target cell of interest, wherein: the target cell of interest promotes or prevents growth, mutagenesis, or metastasis of the tumor or other neoplasm; the target cell of interest is a tumor cell, a cancer cell, or another neoplastic cell; or the target cell of interest is an immune effector cell selected from the group consisting of a T-cell, a B-cell, a dendritic cell, or a natural killer (NK) cell; providing a chimeric protein construct, the chimeric protein construct comprising: a clathrin protein moiety; and a chimeric antigen receptor (CAR) comprising: an ectodomain comprising the antigen binding domain; a transmembrane domain; and an endodomain comprising an intracellular signaling domain; administering the chimeric protein construct to the subject; and inhibiting the growth, mutagenesis, or metastasis of the tumor or other neoplasm or reducing the size or amount of the tumor or other neoplasm.

In some aspects, provided herein are methods of obtaining an image of a target cell of interest in a subject, the method comprising: obtaining an antigenic profile of the target cell of interest; providing an antigen binding domain in response to the antigenic profile of the target cell of interest, wherein the antigen binding domain binds an antigen specific to the target cell of interest, wherein; providing a chimeric protein construct, the chimeric protein construct comprising: a clathrin protein moiety; a chimeric antigen receptor (CAR) comprising: an ectodomain comprising the antigen binding domain; a transmembrane domain; and an endodomain comprising an intracellular signaling domain; and an imaging agent; administering the chimeric protein construct in a detectable amount to the subject; incubating the chimeric protein construct in the subject; detecting the imaging agent; and generating an image of the target cell of interest.

Other objects, features and advantages of the present invention will become clear from the following description and drawings.

### BRIEF DESCRIPTION OF THE FIGURES

FIG. 1 is a schematic example depicting a method of producing a chimeric antigen receptor in vivo. In this embodiment, the patient is administered a chimeric protein construct comprising a clathrin triskelion, a non-clathrin protein moiety comprising chimeric antibody construct for chimeric antigen receptor (CAR) activation. The CL-CM (clathrin-chimeric construct) comprising an ectodomain having a variable heavy chain and variable light chain of scFv; a transmembrane domain; and an endodomain comprising CD28 and CD3-zeta (CD3ζ). On the left, the chimeric protein moiety is taken into a T-cell via endocytosis in a clathrin coated pit, leading to production of the CAR receptor by the T-cell. On the right the chimeric protein construct is taken into a tumor cell via endocytosis in a clathrin coated pit, resulting in a tumor cell with CAR receptors.

FIG. 2 is a schematic depicting a use of bispecific chimeric antigen receptor (CAR) conjugates with Protin-101 (comprising clathrin light chain) for suppression of neuroinflammation and neuronal regeneration. A Protin-101 molecule with a CAR composed of an scFv specific for PD-L1 (Programmed Death Ligand-1) a marker of T regulatory cells (Treg) and on the other end a CAR composed on an scFv specific for Myelin Oligodendrocyte Glycoprotein-1 (MOG-1) a marker of Oligodendrocytes (ODC). Both CARs have the CD28 and CD3 (e.g., CD3-epsilon [CD3-ε] or CD3-zeta [CD3-ζ]) signaling domains. Interaction of the anti-PD-L1 CAR of the conjugate with PD-L1 on Treg will result in the internalization of the conjugate and the trafficking of anti-MOG-1 CAR so that it is exposed on the cell surface of Tregs. Interaction of the anti-MOG-1 CAR of the conjugate with MOG-1 on ODC will result in the internalization of the conjugate and the trafficking of anti-PD-L1 CAR so that it is exposed on the cell surface of ODC. The recognition of MOG-1 on ODC by the anti-MOG-1 CAR on Tregs will result in the activation of Tregs and subsequent immunosuppression in the parenchyma of the central nervous system (CNS) (where ODC are located). Tregs also produce factors that stimulate the differentiation of ODC-precursors into mature ODC and factors that stimulate the production of neural stem cells which in turn results in the differentiation of new neurons. Both activities of Tregs will regenerate damaged neurons. Anti-PD-L1 CAR on the surface of ODC will target ODC to Tregs and so increase the effectiveness of Treg-mediated anti-inflammatory and regenerative effects.

FIG. 3 is a schematic depicting a use of bispecific CAR conjugates with Protin-101 (comprising clathrin light chain) for tumor cell killing. A Protin-101 molecule with a CAR composed of an scFv specific for CD3 a marker of Cytotoxic T cells (CTL) and on the other end a CAR composed of an scFv specific for a tumor protein antigen. Both CARs have the CD28 and CD3 (e.g., CD3-epsilon [CD3-ε] or CD3-zeta [CD3-ζ]) signaling domains. Interaction of the anti-CD3 CAR of the conjugate with CD3 on CTL will result in the internalization of the conjugate and the trafficking of anti-tumor antigen CAR so that it is exposed on the cell surface of CTL. Interaction of the anti-tumor antigen CAR of the conjugate with tumor antigen protein on tumor cells will result in the internalization of the conjugate and the trafficking of anti-CD3 CAR so that it is exposed on the cell surface of tumor cells. The recognition of tumor antigen protein by the anti-tumor antigen protein CAR on CTL will result in killing of the tumor cell by the CTL. Anti-CD3 CAR on the surface of tumor cells will bind to and recruit CTL to the tumor cell further increasing killing by CTL.

FIGS. 4A-4D show the results of assembly and disassembly of clathrin cages in buffer. The protein solution was mixed with 2-(N-morpholino) ethanesulfonic acid (MES) buffer (pH 6.2) at time zero, and the absorbance was read at 320 nm, as shown in the graph (FIG. 4A). Representative transmission electron microscopy (TEM) images of cage assembly of the mixture of Protin-101-light chain and heavy chains show the presence of clathrin cages of different sizes (FIG. 4B). The protein solution was mixed with MES buffer (pH 6.2) at time zero, and the absorbance was read at 320 nm, as shown in the graph, but disassembly of the clathrin cages was induced by addition of 1M tris(hydroxymethyl)aminomethane-HCl (Tris-HCl) buffer (pH 9) after an overall increase of the pH to 9 (FIG. 4C). A representative TEM image of a clathrin cage, disassembled after the increase of the pH to 9 is shown (FIG. 4D).

FIG. 5 shows a series of sequential photographic images depicting the trafficking of clathrin cages in a B6 mouse over the course of 90 minutes. The clathrin light chains were labeled with IR-800 fluorescent dye, then mixed with clathrin heavy chains, and the addition of MES buffer (pH 6.2) was used to synthesize clathrin cages. After concentrating the clathrin cages, 100 microliters (μl) of clathrin concentrate was injected into a C57BL/6 (B6) mouse, and live imaging was performed for 90 minutes (photographs taken at 5 min, 15 min and 30 min [top, left to right respectively] and at 90 min [bottom left] post-injection). The highest level of signal was observed in the liver and the kidneys, followed by the spleen. The organs shown are, left to right: liver, heart, lung, kidney, spleen, pancreas, and intestine (bottom right).

FIGS. 6A-6B are photographic images depicting the biodistribution of free IR800 fluorescent dye alone (FIG. 6A) in comparison to the biodistribution of clathrin cages loaded with IR800 fluorescent dye (FIG. 6B). IR800 fluorescent dye was loaded into clathrin cages and injected into C57BL/6 (B6) mice. As a control, IR800 fluorescent dye was injected directly into C57BL/6 control mice. Mice were sacrificed 24 hours post-injection, and the major organs and lymph nodes were imaged (FIGS. 6A-6B).

FIGS. 7A-7B are graphs demonstrating the purification of anti-PD-1 conjugated with Prot101 (labeled by ALEXA FLUOR® 594 by maleimide chemistry). Following purification by a 50 kDa centrifugal membrane filter (10,000 rpm, 5 min, concentration factor of 10), the first, second, third, and fourth filtrates after dialysis show no significant absorbance from the ALEXA FLUOR® 594 label (Alexa594), indicating that the conjugation yield approaches 100% (FIG. 7A). With respect to the retentates, the absorbance from ALEXA FLUOR® 594 decreases with increasing rounds of dialyses (first, second, third, and fourth), which arises from the absorption to the membrane (FIG. 7B), possibly originating from a decrease in stability of the proteins (aggregation).

FIG. 8 shows a series of sequential photographic images depicting the trafficking of clathrin light chains (Protin-101) in a B6 mouse over the course of 90 minutes. The clathrin light chains were labeled with IR-800 fluorescent dye. The clathrin light chains were then injected into a C57BL/6 (B6) mouse, and live imaging was performed for 90 minutes (photographs taken at 3 min and 15 min [top, left to right, respectively] and at 30 min and 90 min [middle, left to right, respectively] post-injection). The highest level of signal was observed in the liver and the kidneys (indicated by the dashed red ovals), followed by the spleen. The organs shown are, left to right: liver, heart, lung, kidney, spleen, pancreas, and intestine (bottom).

FIG. 9 shows a series of photographic images depicting the trafficking of clathrin heavy chains (Protin-102) in a B6 mouse. The clathrin heavy chains were labeled with CF680 dye. The clathrin heavy chains were then injected into a C57BL/6 (B6) mouse, and imaging was performed 24 hours post-injection. On the left, the photographic image shows ex vivo images of the concentration in the mesenteric lymph nodes (left). The highest level of signal was observed in the liver (top right). Further review showed the concentration in the mesenteric lymph nodes (Mes) as indicated by the oval red dashed (bottom right), as compared with the axillary (Ax) and inguinal (Ing) lymph nodes.

FIG. 10 shows a series of photographic images depicting stained left (left) and right (right) kidneys in a B6 mouse. The green regions indicate podoplanin (PDPN), which was stained. The blue regions are DNA stained with 4′,6-diamidino-2-phenylindole (DAPI), a fluorescent stain that binds strongly to adenine-thymine (AT)-rich regions in DNA. The smaller red spots are labeled Protin-101 (light chain) (Prot101).

FIG. 11 shows a photographic image depicting histological examination of mesenteric lymph nodes (MLN) and showing trafficking of Protin-101 (Prot 101) (red spots, orange arrows) to the MLN in a B6 mouse.

FIG. 12 shows a photographic image depicting histological examination of mesenteric lymph nodes (MLN) and showing trafficking of Protin-101 (Prot 101) (red spots) to the MLN in a C57BL/6 mouse. Green staining indicates dye specific for lymphatic vessel endothelial hyaluronan receptor 1 (LYVE1), also known as extracellular link domain containing 1 (XLKD1). LYVE1 is a Link domain-containing hyaladherin, a protein capable of binding to hyaluronic acid (HA), homologous to CD44, the main HA receptor.

FIG. 13 shows a series of sequential photographic images depicting the trafficking of clathrin cages in a B6 mouse over the course of 20 minutes. The clathrin cages were labeled. The clathrin cages were then injected into a C57BL/6 (B6) mouse 8 days after tumor implantation in the left kidney, and live imaging was performed for 20 minutes (photographs taken at 3 min and 5 min [top, left to right, respectively], and at 10 min and 20 min [bottom, left to right, respectively] following injection). The highest level of signal was observed as indicated in the ovals.

FIGS. 14A-14F shows a series of graphs and photographic images depicting the therapeutic effects of Prot101-TAXOL® conjugates (Protin-101 [clathrin light chains]-TAXOL® conjugates) (PTCs). PTC significantly inhibited the progress of primary tumor and lung metastasis without side effects in 4T1 murine breast cancer model. Therapeutic effects of Prot101-TAXOL® conjugates (Protin-101 [clathrin light chains]-TAXOL® conjugates; PTCs) were studied in a xenograft BALB/c mouse model bearing 4T1 murine breast cancer, which is refractory to many chemotherapeutics. On day 13 post-implant of 1×10⁵ 4T1 cells when tumor volume reaches ˜100 mm3, the mice were randomly divided into 3 groups, based on the size of tumor, and were intravenously administrated by the following formulations 2 times per week for 2 weeks: PBS, free TAXOL®, and PTC. FIG. 14A is a graph showing growth curves of 4T1 tumors after implant. Treatment was started on day 13-post implant of tumor, and drugs were intravenously administrated by 2 times per week for 2 weeks (n=6/group). On day 27 post-implant, all the mice were euthanized because the tumor in the mice treated with PBS reached ˜2 cm of diameter. FIG. 14B shows representative photographs of tumors (top) and lungs (bottom) in each group. FIG. 14C is a graph depicting body weight of 4T1 tumor-bearing mice in each group after intravenous administration (n=6/group). FIGS. 14D-14E are graphs depicting growth of curves of B16 melanoma (FIG. 14D) and Lewis lung carcinoma line 1 (LLC1) (FIG. 14E) tumors after implant. The drugs were injected on the identical schedule with the 4T1 study. FIG. 14F shows representative photographs of tumors of B16 (top) and LLC1 (bottom). Scale bar; 1 cm. Arrows; the treated days. Red circle; tumor. White circle; nodules. **p<0.01, ***p<0.001. Asterisks indicate significant differences between the mice treated with PTC and the mice treated with others.

### DETAILED DESCRIPTION

Previous work in this area has focused on methods of treating cancer with CAR T-cells by extracting the patient's immune T-cells, genetically engineering the T-cell to produce chimeric antigen receptors (CARs), expanding the modified T-cell and infusing the patient with the product. However, the necessary manipulations of the patient's immune system are associated with high costs, as well as significant toxicity and side effects that can be fatal, including massive toxin release (cytokine release syndrome [CRS]), B cell aplasia (large-scale B cell death), cerebral edema, and neurotoxicities.

Lymph nodes (LN) are a critical cite of pathogenesis in immune-mediated diseases and cancer and are critical sites of targeting delivery of immunoregulatory molecules, check point inhibitors, and chemotherapy drugs. LN targeted delivery can markedly augment the therapeutic index of therapeutics, increasing their efficacy while reducing their toxicity. Other previous work in this area has also relied on lymphatic absorption via skin injection of the therapeutics directly.

It is desirable to provide an improved alternative to the present methods of generating CAR T-cells for the detection or treatment of cancer. It is also desirable to provide more systemic, targeted delivery methods, e.g., to lymph nodes and other targets of interest.

Provided herein are improved chimeric protein constructs and methods of generating CAR T-cells, CAR B-cells and CAR Treg-cells and other CAR-engineered cells in vivo in a subject in need thereof. These methods can be used for greater accuracy in many fields, including, but not limited to, imaging, biomarking, and medical applications, providing increased multifunctionality, reduced toxicity, enhanced solubility, improved bioavailability, more specific agent/drug/etc. targeting and delivery, prolonged circulation lifetimes, decreased drug resistance, and fewer side effects. Further, cost reduction and drug availability as well as ease of treatment as compare to the complexity of the existing technology will increase the utility of CAR-engineered therapy.

Here, drug preparation of a fusion or attachment of endogenous clathrin, (light and/or heavy chains, either as peptides, or as a clathrin cage, barrel, or basket, or as attached to one or more CAR constructs for in vivo T-cells, B-cells and Treg cells are disclosed. Attachments to the clathrin moieties can occur either as attachment to clathrin light chains and/or heavy chains or as attachment after the clathrin moieties are assembled. In some embodiments light chains are attached separately. In some embodiments, heavy chains are attached separately. In some embodiments, only light chains are used. In some embodiments, only heavy chains are used. In some embodiments, both light chains and heavy chains are used. In some embodiments, for example, a light chain and a heavy chain could be attached separately or could be self-assembled prior to attachment. In some embodiments, they are fused with other proteins or chemically attached to a CAR construct to activate T and other immune cells. In other embodiment, clathrin is attached via a tether or linker or protein to CAR constructs or therapy payload, such as a chemotherapeutic drug, antibody, and/or enzyme inhibitor. In some embodiments, several variants, including chimeric protein constructs, are disclosed (fusions or chemically attached to a protein) for activating T cells, B cells and other immune cells in order to home in on and bind to specific antigens that recognize and destroy cancer cells or to treat autoimmune, inflammation and neuroinflammatory diseases. Further, these endogenous proteins such as clathrin light chain target lymph node venules. For example, the protein in in vivo trafficking of human pancreatic cancer implanted in nude mice exhibited a super high ratio of tumor cells killed in comparison to the collateral damage in the surrounding normal cells. Essentially, more tumor cells are killed, compared to the non-tumor cells, by having a higher concentration of payload target the tumor cells as opposed to any collateral damage to the non-tumor cells. More tumor cells are killed by having a higher concentration of CAR constructs targeting them as compared to collateral damage to the surrounding normal cells. These high tumor concentrations lower the toxic effect of normal cells and allow more effective tumor treatment. The lymph node venule concentration allows specific drug delivery to a tumor and metastasis via the lymphatic system. This protein fusion provides a chimeric protein to target specific T cell, B cell, Treg-cell or other cluster of differentiation (CD) proteins will enable cell in vivo immune system activation and targeted in vivo cell killing following their administration.

The advantages of this technology over traditional CAR T-cell methods are significant. First, no patient's cell extraction is required. Second. no cell expansion is required. Third, T Cell and B cell or Treg-cell activation are performed in vivo. Fourth, the protein is endogenous, and its fusion or combination with the chimeric protein are designed to target the specific antigen. Fifth, there is a decreased risk of massive cytokine release syndrome, and therefore fewer fatal side effects are expected. Sixth, the technology manipulations needed for the injectable final drug product, significantly decrease the planning, administration, and cost per treatment of this in vivo cell therapy and finally it allows more widespread utility.

In some aspects, provided herein are chimeric protein constructs comprising: a clathrin protein moiety or a functional derivative thereof; and a chimeric antigen receptor (CAR) comprising: an ectodomain comprising at least one antigen binding domain; a transmembrane domain; and an endodomain comprising an intracellular signaling domain.

In some embodiments, the CAR is linked, conjugated, bound, tethered, or fused to the clathrin protein moiety.

In some embodiments, the clathrin protein moiety and the CAR comprise a fusion protein.

In some embodiments, the chimeric protein construct further comprises a protein linker operably linking the clathrin protein moiety and the CAR.

In some embodiments, the clathrin protein moiety comprises a clathrin light chain or a clathrin heavy chain or a modified analog thereof. In some embodiments, the clathrin protein moiety comprises a clathrin light chain and a clathrin heavy chain. In some embodiments, the clathrin heavy chain at least 95% identical to SEQ ID NO: 1 or to SEQ ID NO: 3. In some embodiments, the clathrin light chain at least 95% identical to SEQ ID NO: 2 or to SEQ ID NO: 4.

In some embodiments, the clathrin protein moiety comprises a clathrin cage. In some embodiments, the clathrin protein moiety comprises a three-dimensional clathrin cage structure comprising an outer surface and an inner cavity, the CAR conjugated, bound, linked, tethered, or fused to the outer surface of the three-dimensional clathrin cage structure.

In some embodiments, the chimeric protein construct further comprises a payload. In some embodiments, the payload conjugated, bound, linked, tethered, or fused to the clathrin protein moiety or to the CAR. In some embodiments, the clathrin protein moiety comprises a three-dimensional clathrin cage structure comprising an outer surface and an inner cavity, the CAR conjugated, bound, linked, tethered, or fused to the outer surface of the three-dimensional clathrin cage structure and the payload conjugated, bound, linked, tethered, fused, or at least partially contained within the inner cavity of the clathrin cage structure.

In some embodiments, the payload comprises a medicament, an imaging agent, or a biomarker. In some embodiments, the imaging agent comprises a fluorescence, a radionuclide or an MRI contrast agent.

In some embodiments, the payload comprises a medicament or other pharmaceutical agent for treating or alleviating a disease or an abnormal physiological condition. In some embodiments, the disease or abnormal physiological condition comprises a tumor, a cancer, a neurodegenerative disease or condition, an autoimmune disease, a transplant rejection, an inflammatory or neuroinflammatory disease or condition, or an infectious disease.

In some embodiments, the medicament or other pharmaceutical agent comprises: gemcitabine or a functional derivative thereof; paclitaxel or a functional derivative thereof; docetaxel or a functional derivative thereof; carboplatin or a functional derivative thereof; cisplatin or a functional derivative thereof; azonafide or a functional derivative thereof; pembrolizumab or a functional derivative thereof; nivolumab or a functional derivative thereof; cemiplimab or a functional derivative thereof; pidilizumab or a functional derivative thereof; BMS-926559 or a functional derivative thereof; atezolizumab or a functional derivative thereof; avelumab or a functional derivative thereof; durvalumab or a functional derivative thereof; ipilimumab or a functional derivative thereof; an anti-programmed cell death protein 1 (anti-PD-1) antibody or antibody derived protein, or an anti-PD-1 antigen-binding domain; an anti-programmed death-linker 1 (anti-PD-L1) antibody or antibody derived protein, or an anti-PD-L1 antigen-binding domain; or an anti-cytotoxic T-lymphocyte-associated protein 4 (anti-CTLA4) antibody or antibody derived protein, or an anti-CTLA4 antigen-binding domain; or any combination thereof.

In some embodiments, the payload comprises a pharmaceutical composition, an antibody, an antibody-drug conjugate, a nucleic acid, a protein, a peptide, or a polypeptide or polynucleotide vector. In some embodiments, the payload comprising an antibody-drug conjugate, the antibody or antigen-binding domain thereof recognizing or binding to an antigen on a tumor cell or neoplastic cell, a cancer cell, a cell that promotes growth, mutagenesis, or metastasis of a tumor cell or other neoplastic cell, a neural cell, or an innate immune cell; and the drug thereof comprising a treatment drug.

In some embodiments, the endodomain further comprises: at least one costimulatory domain; at least one nuclear factor of activated T cell, B Cell-responsive inducible expression element; or a combination thereof.

In some embodiments, the ectodomain further comprises a transport signal peptide.

In some embodiments, the CAR further comprises at least one additional intracellular signaling domain.

In some embodiments, the at least one antigen-binding domain of the CAR recognizing or binding to an antigen specific to a target cell of interest. In some embodiments, the target cell of interest comprises: a T-cell; a B-cell; a regulatory T (Treg) cell; a mutant cell; a diseased cell; a tumor cell or neoplastic cell; a cancer cell; a cell that promotes growth, mutagenesis, or metastasis of a tumor cell or other neoplastic cell; a cell expressing a specific marker of interest; a regulatory cell; an innate immune cell; a neural cell; a glial cell; a secretory cell; a cell that inhibits or promotes cell death; an immune effector cell; a cell that regulates an immune effector cell; a cell belonging to an infectious agent; or an immunosuppressive cell.

In some embodiments, the at least one antigen-binding domain of the CAR recognizes or binds to an antigen on an immune effector cell, a lymphoid cell, a cell that regulates an immune effector cell, a regulatory cell, a lymphoid cell, a secretory cell, or a cell that inhibits or promotes cell death.

In some embodiments, the target cell of interest is an immune effector cell selected from the group consisting of a T-cell, a regulatory T (Treg) cell, a B-cell, a dendritic cell, or a natural killer (NK) cell.

In some embodiments, the target cell of interest comprises a pancreatic ductal adenocarcinoma (PDA) cell, a lymphocyte, a gamma-delta T-cell (γδ-T-cell), a lymph node venule cell, a breast tumor or cancer cell, a renal tumor or carcinoma cell, a skin tumor, a melanoma cell, a bladder tumor or cancer cell, a gastric tumor or cancer cell, a lung tumor, a non-small cell lung cancer cell, a lymphoma cell, a mesothelioma cell, a urothelial carcinoma cell, a Merkel-cell carcinoma cell, a head or neck cancer cell, a squamous cell carcinoma cell, a Treg cell, a neural cell, an innate immune cell, an inflammatory cell, or a disease modulating cell.

In some embodiments, the antigen comprises a proinflammatory or anti-inflammatory cytokine. In some embodiments, the antigen comprises interleukin-10 (IL-10), interleukin-2 (IL-2), interleukin-6 (IL-6) or another interleukin; transforming growth factor beta (TGF-0); programmed cell death protein 1 (PD-1); programmed death-ligand 1 (PD-L1); or cytotoxic T lymphocyte-associated protein 4 (CTLA-4).

In some embodiments, the target cell of interest comprises a lymphoid cell.

In some embodiments, the antigen comprises L-selectin.

In some embodiments, the antigen-binding domain of the CAR comprises an anti-peripheral lymph node addressin (PNAd) binding domain.

In some embodiments, the antibody or antigen binding domain thereof recognizes or binds to an antigen on a tumor cell or neoplastic cell, a cancer cell, or a cell that promotes growth, mutagenesis, or metastasis of a tumor cell or other neoplastic cell.

In some embodiments, the ectodomain of the CAR comprises two antigen-binding domains, each recognizing or binding to a different antigen. In some embodiments, the ectodomain of the CAR comprises two antigen-binding domains, wherein a first antigen-binding domain recognizes or binds to an antigen on a first target cell of interest and a second antigen-binding domain recognizes or binds to an antigen on a second target cell of interest.

In some embodiments, the first target cell of interest comprises an immune effector cell, a cell that regulates an immune effector cell, a regulatory cell, a lymphoid cell, secretory cell, or a cell that inhibits or promotes cell death.

In some embodiments, the second target cell of interest comprises a tumor cell or neoplastic cell, a cancer cell, or a cell that promotes growth, mutagenesis, or metastasis of a tumor cell or other neoplastic cell, a mutant cell, a diseased cell, or a cell belonging to an inflammatory or infectious agent. In some embodiments, the second target cell of interest comprises a lymphoid cell.

In some embodiments, the antigen binding domain comprises an antigen-binding single-chain Fv (scFv).

In some embodiments, the endodomain comprises CD28 or a costimulatory domain thereof, CD137 (4-1BB) or a costimulatory domain thereof, or CD134 (OX40) or a costimulatory domain thereof.

In some embodiments, the chimeric protein construct further comprises an antibody or an antigen-binding fragment thereof, the antibody or antigen-binding fragment thereof comprising an antigen-binding domain distinct from the antigen-binding domain of the CAR and recognizing or binding to an antigen of interest. In some embodiments, the antibody or antigen-binding fragment thereof recognizing or binding to a secretory protein or to a cell surface protein. In some embodiments, the antibody comprising an antibody-drug conjugate comprising an antibody or an antigen binding domain thereof linked to a drug. In some embodiments, the antibody or antigen-binding domain thereof recognizing or binding to an antigen on a tumor cell or neoplastic cell, a cancer cell, or a cell that promotes growth, mutagenesis, or metastasis of a tumor cell or other neoplastic cell; and the drug comprising a cytotoxic drug.

In some aspects, provided herein are methods of producing a chimeric antigen receptor (CAR)-engineered cell of interest in vivo, the method comprising: providing a chimeric protein construct comprising: a clathrin protein moiety or a functional derivative thereof; and a chimeric antigen receptor (CAR) comprising: an ectodomain comprising at least one antigen binding domain; a transmembrane domain; and an endodomain comprising an intracellular signaling domain; administering the chimeric protein construct to a subject in need thereof; and transducing a target cell of interest in the subject with the chimeric protein construct to produce a CAR-engineered cell of interest.

In some embodiments, the step of transducing the target cell of interest with the chimeric protein construct comprises clathrin-mediated endocytosis of the CAR or the chimeric protein construct.

In some embodiments, the target cell of interest is an immune effector cell selected from the group consisting of a T-cell, a regulatory T (Treg) cell, a B-cell, a dendritic cell, or a natural killer (NK) cell. In some embodiments, the target cell of interest is an immune modulating cell. In some embodiments, the target cell of interest is a tumor cell or neoplastic cell; a cancer cell; or a cell that promotes growth, mutagenesis, or metastasis of a tumor cell, a neoplastic cell, or a cancer cell; or a pro-inflammatory cell, an anti-inflammatory cell, or an infectious cell. In some embodiments, the target cell of interest comprises a pancreatic ductal adenocarcinoma (PDA) cell, a lymphocyte, a gamma-delta T-cell (γδ-T-cell), a lymph node venule cell, a breast tumor or cancer cell, a renal tumor or carcinoma cell, a skin tumor, a melanoma cell, a bladder tumor or cancer cell, a gastric tumor or cancer cell, a lung tumor, a non-small cell lung cancer cell, a lymphoma cell, a mesothelioma cell, a urothelial carcinoma cell, a Merkel-cell carcinoma cell, a head or neck cancer cell, a squamous cell carcinoma cell, a Treg cell, a neural cell, an innate immune cell, an inflammatory cell, or a disease modulating cell.

In some embodiments, the CAR is linked to the clathrin protein moiety. In some embodiments, the CAR is conjugated, bound, tethered, or fused to the clathrin protein moiety. In some embodiments, the clathrin protein moiety and the CAR comprise a fusion protein.

In some embodiments, the clathrin protein moiety comprising a clathrin light chain, a clathrin heavy chain, or a combination thereof. In some embodiments, the clathrin heavy chain is at least 95% identical to SEQ ID NO: 1 or to SEQ ID NO: 3. In some embodiments, the clathrin light chain at least 95% identical to SEQ ID NO: 2 or to SEQ ID NO: 4.

In some embodiments, the clathrin protein moiety comprises a clathrin cage. In some embodiments, the clathrin protein moiety further comprises a payload. In some embodiments, the payload is conjugated, bound, linked, tethered, or fused to the clathrin protein moiety or to the CAR. In some embodiments, the clathrin protein moiety comprises a three-dimensional clathrin cage structure comprising an outer surface and an inner cavity, the CAR conjugated, bound, linked, tethered, or fused to the outer surface of the three-dimensional clathrin cage structure and the payload conjugated, bound, linked, tethered, fused, or at least partially contained within the inner cavity of the clathrin cage structure.

In some embodiments, the payload comprises an imaging agent, a biomarker, or a medicament or other pharmaceutical agent.

In some embodiments, the antigen binding domain comprises an antigen-binding single-chain Fv (scFv). In some embodiments, the antigen-binding scFv comprises a heavy chain and a light chain operably linked by an scFv linker.

In some aspects, provided herein are methods of inhibiting the growth, mutagenesis, or metastasis of a cancer, a tumor, or other neoplasm in a subject in need thereof, the method comprising: obtaining an antigenic profile of the cancer, the tumor, or the other neoplasm; providing an antigen binding domain in response to the antigenic profile of the cancer, the tumor, or the other neoplasm, wherein the antigen binding domain binds an antigen specific to the tumor or other neoplasm or an antigen specific to a target cell of interest, wherein: the target cell of interest promotes or prevents growth, mutagenesis, or metastasis of the tumor or other neoplasm; the target cell of interest is a tumor cell, a cancer cell, or another neoplastic cell; or the target cell of interest is an immune effector cell selected from the group consisting of a T-cell, a B-cell, a dendritic cell, or a natural killer (NK) cell; providing a chimeric protein construct, the chimeric protein construct comprising: a clathrin protein moiety; and a chimeric antigen receptor (CAR) comprising: an ectodomain comprising the antigen binding domain; a transmembrane domain; and an endodomain comprising an intracellular signaling domain; administering the chimeric protein construct to the subject; and inhibiting the growth, mutagenesis, or metastasis of the tumor or other neoplasm or reducing the size or amount of the tumor or other neoplasm.

In some aspects, provided herein are methods of obtaining an image of a target cell of interest in a subject, the method comprising: obtaining an antigenic profile of the target cell of interest; providing an antigen binding domain in response to the antigenic profile of the target cell of interest, wherein the antigen binding domain binds an antigen specific to the target cell of interest, wherein; providing a chimeric protein construct, the chimeric protein construct comprising: a clathrin protein moiety; a chimeric antigen receptor (CAR) comprising: an ectodomain comprising the antigen binding domain; a transmembrane domain; and an endodomain comprising an intracellular signaling domain; and an imaging agent; administering the chimeric protein construct in a detectable amount to the subject; incubating the chimeric protein construct in the subject; detecting the imaging agent; and generating an image of the target cell of interest.

In certain embodiments, this invention relates to the use of self-assembling protein molecules combined with a specific engineered CAR activating T-cells, for example, a CAR construct that expresses CD28 and CD3 (e.g., CD3-epsilon or CD3-zeta), -but is not limited to this scFv construct, for activating T-cells in vivo for cancer treatment.

In certain embodiments, the protein is clathrin or a derivative of clathrin. In certain embodiments, the protein is endogenous and/or non-immunogenic. In certain embodiments, the protein is ferritin or a derivative of ferritin.

In some embodiments, the self-assembled protein cages or vehicles, made of heavy and/or light chains, mask the toxicity of the anti-cancer agent, thereby resulting in decreased serum and systemic toxicity.

In certain embodiments, the heavy chain and the light chain are gathered as one delivery system or fused (e.g., the protein may be a fusion protein). In certain embodiments, heavy chains are gathered as one delivery system or fused (e.g., as a fusion protein) and/or light chains are gathered as one delivery system or fused (e.g., as a fusion protein).

In other embodiments, the construct is used to target specific tissues, such as cancer cells, using antigen biomarkers, antibodies, or peptides that are recognized by the cell membrane of the target cell. In certain embodiments, once delivered to the target tissues, the clathrin cages are internalized by the cell for in-cell deposition of, e.g., a drug or other payload.

In certain embodiments, the payload is an anti-cancer agent, for example, a chemotherapeutic, an siRNA, an miRNA, an immunotherapeutic, or a radiotherapeutic. In some embodiments, the payload is an imaging agent, such as a contrast medium or a fluorophore, in certain embodiments, the drug is a radiotherapeutic, such as a radionuclide.

In certain embodiments, the CAR is conjugated to the protein, for example, to the clathrin light chain or to the clathrin heavy chain. “Conjugated” as used herein means ionically or, preferably, covalently attached (e.g., via a crosslinking agent).

In certain embodiments, the invention relates to a method of producing a delivery of CAR-engineered to target immune cell activation in vivo for specific disease treatment. In certain embodiments, the method delivers an imaging agent, biomarker, or medicament to a cell.

In certain embodiments, the invention relates to a method of treating a subject in need thereof comprising administering to the subject a therapeutically effective amount of any one of the delivery protein (i.e., clathrin)-CAR engineered cells for treatment. In certain embodiments, these drug-constructs are administered to the subject intravenously, intraperitoneally or intratumorally.

This technology is expected to achieve synergistic results as compared to the protein alone, the payload alone, the CAR alone, or any combination of two of these components. The advantages include, but are not limited to: 1. The CAR engineered proteins attached to self-assemble proteins. 2. The proteins are easily internalized by cells. 3. The assembled, drug-constructs are stable in serum proteins and are non-toxic while transported in vivo via the blood and lymph system. 4. The proteins and vehicle drug constructs are designed to specifically target diseased cells using specific antibodies or high-affinity fragments of antibodies. In some embodiments, the antibodies are designed to enhance the immune system by uncovering a cancer call not identified by the immune system. 5. Once targeted to immune cells, the constructs may internalize to activate the cells that specifically kill the diseased cell or allow the immune system to fight it. 6. This platform has the potential to provide mono-, bi- and multi-specific targeting. 7. Because of the clathrin ability to internalize, imaging agent or a radiotherapeutic could be attached and may be used for tumor imaging or radiotherapy. 8. For therapeutic applications where longer half-life is desired, the vehicles may be modified by increasing the molecular weight of the proteins or adding polymeric extensions. 9. The combination of (i) endogenous, self-assembled, cell-internalized proteins with (ii) self-internalized antibodies and (iii) payloads can improve cancer imaging or treatment while lowering systemic toxicity.

In certain embodiments, the invention also relates to any of the compositions described herein, wherein the composition is a cell-specific therapeutic and/or imaging-agent delivery system. Targeted therapeutic delivery systems can enhance the effective dose at the site, such as a tumor or neuroinflammation, while decreasing general exposure to the drug and its associated side effects.

In some embodiments, the CAR comprises at least one antigen-binding domain. In some embodiments, the CAR has one antigen-binding domain for a surface protein on a target cell of interest. In some embodiments, the CAR has one antigen-binding domain for an imaging agent, a biomarker, or a pharmaceutical agent. In some embodiments, the CAR has more than one antigen-binding domain, and at least one antigen-binding for a surface protein on a target cell of interest and/or an imaging agent, a biomarker, or a pharmaceutical agent. In some embodiments, the CAR has more than one antigen-binding domain, e.g., each for a different epitope of a given surface protein on a target cell of interest, each for one or more surface proteins on a target cell of interest, each for a different surface protein on each of two different target cells of interest, or one for a surface protein on a target cell of interest and the other for an imaging agent, a biomarker, a pharmaceutical agent, or an enzyme. For example, a subsequent CAR antigen-binding domain may target a different antigen on the same target, or a subsequent CAR antigen-binding domain may target an antigen on a different target (e.g., to bring different targets into proximity with each other).

In some embodiments, the chimeric protein construct additionally comprises an antibody or a moiety having antigen-binding domain distinct from the at least one antigen-binding domain(s) of the CAR, and this antibody or non-CAR antigen-binding domain provides additional selectively or provides an additional binding domain for an imaging agent, a biomarker, a pharmaceutical agent, or an enzyme. In embodiments in which the CAR has at least one antigen binding domain targeting a cell of interest, this non-CAR antibody or antigen-binding-domain may target a different antigen on the same target or this non-CAR antibody or antigen-binding domain may target an antigen on a different target (e.g., to bring different targets into proximity with each other).

Also provided herein are variations, including the following chimeric protein construct forms including, but not limited to: scFv constructs comprising CD28, CD3 (e.g., CD3-epsilon or CD3-zeta) and or other modified versions, typical or universal. Additional variations include, but are not limited to:

A. Clathrin light chain-type chimeric protein construct: a clathrin light chain is bound to the CAR through a tether or fusion.

B. Clathrin light chain (self-assembled)-type chimeric protein construct: a self-assembled clathrin light chain is bound to the CAR through a tether or fusion.

C. Clathrin heavy chain-type chimeric protein construct: a clathrin heavy chain is bound to the CAR through a tether or fusion.

D. Clathrin heavy chain (self-assembled)-type chimeric protein construct: a self-assembled clathrin heavy chain is bound to the CAR through a tether or fusion.

E. Clathrin light chain and heavy chain (self-assembled)-type chimeric protein construct: a self-assembled clathrin light chain and heavy chain combination is bound to the CAR through a tether or fusion.

F. Clathrin light and/or heavy chain-type chimeric protein construct: a clathrin light chain and/or heavy chain is bound to the CAR through a tether or fusion.

G. Clathrin light and/or heavy chain (self-assembled)-type chimeric protein construct: a self-assembled clathrin light chain and/or heavy chain is bound to the CAR through a tether or fusion.

H. According to some embodiments, treatment with a chimeric protein construct of the present invention is used in combination with other cancer therapy treatments, including, but not limited to chemotherapies, surgery, or radiotherapy. Other cancer therapy treatments are administered in prior to, in parallel, concurrently, or subsequently with respect to treatment with a chimeric protein construct of the present invention.

### Clathrins and Derivative Proteins

In some embodiments, the clathrin protein moiety or functional derivative thereof is a clathrin light chain and/or a clathrin heavy chain. Alternatively, it is a clathrin triskelion or a clathrin cage structure (including, but not limited to, a clathrin cage, a clathrin barrel, or a clathrin basket).

In some embodiments, the clathrin triskelion or the clathrin cage structure is self-assembled, namely, the clathrin protein(s) is exposed to conditions in which the triskelion or the cage structure assembles. In some embodiments, clathrin molecules exposed to, e.g., a buffer or biological fluid at an appropriate pH can be induced to assemble as a triskelion attached to the non-clathrin moiety (i.e., chimeric antibody), or to assemble as a three-dimensional clathrin structure (e.g., a cage, etc.) surrounding or enclosing all or part of the non-clathrin moiety (i.e., chimeric antibody). Alternatively, exposure to a different pH (e.g., a biological fluid or cellular microenvironment) can induce the complex clathrin structure to disassemble into its clathrin components, releasing the non-clathrin moiety (i.e., chimeric antibody), where the non-clathrin moiety (i.e., chimeric antibody), is inside a cage, barrel, or basket.

In some embodiments, clathrin molecules exposed to, e.g., a buffer or biological fluid at an appropriate pH can be induced to assemble as a triskelion attached to the non-clathrin moiety (i.e., chimeric antibody), or to assemble as a three-dimensional clathrin structure (e.g., a cage, etc.) surrounding or enclosing all or part of a payload (i.e., chimeric antibody). Alternatively, exposure to a different pH (e.g., a biological fluid or cellular microenvironment) can induce the complex clathrin structure to disassemble into its clathrin components, releasing the payload, such as chimeric antibody, or an additional chemotherapy or other antibody treatment (e.g., where attached to a clathrin triskelion) or releasing or exposing the payload (e.g., where the non-clathrin moiety is inside a cage, barrel, or basket).

In some embodiments, an adaptor protein is used for self-assembly. Examples of adaptor proteins include, but are not limited to anti-PD-1, anti PD-L1, anti-AP180 and/or anti-epsin.

In some embodiments, clathrin is attached to anti-PD-1 and/or anti-PD-L1 antibody fragments, chemotherapy, CAR constructs, or an antibody or construct comprising an antigen-binding domain; a pharmaceutical compound; an antibody-drug conjugate; a biomarker; or an imaging agent at pH 6-7 to induce assembly into a clathrin cage, including a clathrin cage enclosing, e.g., anti-PD-1, anti-PD-L1, chemotherapy, a CAR construct, or an antibody or construct comprising an antigen-binding domain; a pharmaceutical compound; an antibody-drug conjugate; a biomarker; or an imaging agent.

In some embodiments, the clathrin cage comprises a mini-coat (e.g., having 12 pentagons and 2 hexagons. In some embodiments, the clathrin cage comprises a mini-coat having tetrahedral symmetry (e.g., having 12 pentagons and 4 hexagons). In some embodiments, the clathrin cage comprises a hexagonal barrel (e.g., having 8 hexagons, 12 pentagons, and D6 symmetry). In some embodiments, the clathrin cage comprises a soccer ball (having 12 pentagons, 20 hexagons, and icosahedral symmetry, such as in a truncated icosahedron). In some embodiments, the clathrin comprises a triskelion.

In some embodiments, the clathrin moiety is attached to an antibody specifically recognizing a tumor or cancer cell surface in order to target a tumor or cancer cell specifically. In other embodiments, it has been found that clathrin is preferentially directed to a tumor or cancer cell without the need to add a tumor cell surface targeting antibody. This approach has been accomplished via the mesenteric lymph system.

In some embodiments, the clathrin heavy chain is a clathrin heavy chain having SEQ ID NO: 1:

(SEQ ID NO: 1)
Met Ala Gln Ile Leu Pro Ile Arg Phe Gln Glu His Leu Gln Leu Gln Asn Leu Gly Ile Asn Pro Ala
Asn Ile Gly Phe Ser Thr Leu Thr Met Glu Ser Asp Lys Phe Ile Cys Ile Arg Glu Lys Val Gly Glu
Gln Ala Gln Val Val Ile Ile Asp Met Asn Asp Pro Ser Asn Pro Ile Arg Arg Pro Ile Ser Ala Asp
Ser Ala Ile Met Asn Pro Ala Ser Lys Val Ile Phe Asn Ile Glu Met Lys Ser Lys Met Lys Ala His
Thr Met Thr Asp Asp Val Thr Phe Trp Lys Trp Ile Ser Leu Asn Thr Val Ala Leu Val Thr Asp Asn
Ala Val Tyr His Trp Ser Met Glu Gly Glu Ser Gln Pro Val Lys Met Phe Asp Arg His Ser Ser Leu
Ala Gly Cys Gln Ile Ile Asn Tyr Arg Thr Asp Ala Ala Leu Lys Ala Gly Lys Thr Leu Gln Ile Lys
Gln Lys Trp Leu Leu Leu Thr Gly Ile Ser Ala Gln Gln Asn Arg Val Val Gly Ala Met Gln Leu Tyr
Ser Val Asp Arg Lys Val Ser Gln Pro Ile Glu Gly His Ala Ala Ser Phe Ala Gln Phe Lys Met Glu
Gly Asn Ala Glu Glu Ser Thr Leu Phe Cys Phe Ala Val Arg Gly Gln Ala Gly Gly Lys Leu His Ile
Ile Glu Val Gly Thr Pro Pro Thr Gly Asn Gln Pro Phe Pro Lys Lys Ala Val Asp Val Phe Phe Pro
Pro Glu Ala Gln Asn Asp Phe Pro Val Ala Met Gln Ile Ser Glu Lys His Asp Val Val Phe Leu Ile
Thr Lys Tyr Gly Tyr Ile His Leu Tyr Asp Leu Glu Thr Gly Thr Cys Ile Tyr Met Asn Arg Ile Ser
Gly Glu Thr Ile Phe Val Thr Ala Pro His Glu Ala Thr Ala Gly Ile Ile Gly Val Asn Arg Lys Gly
Gln Val Leu Ser Val Cys Val Glu Glu Glu Asn Ile Ile Pro Tyr Ile Thr Asn Val Leu Gln Asn Pro
Asp Leu Ala Leu Arg Met Ala Val Arg Asn Asn Leu Ala Gly Ala Glu Glu Leu Phe Ala Arg Lys
Phe Asn Ala Leu Phe Ala Gln Gly Asn Tyr Ser Glu Ala Ala Lys Val Ala Ala Asn Ala Pro Lys
Gly Ile Leu Arg Thr Pro Asp Thr Ile Arg Arg Phe Gln Ser Val Pro Ala Gln Pro Gly Gln Thr Ser
Pro Leu Leu Gln Tyr Phe Gly Ile Leu Leu Asp Gln Gly Gln Leu Asn Lys Tyr Glu Ser Leu Glu
Leu Cys Arg Pro Val Leu Gln Gln Gly Arg Lys Gln Leu Leu Glu Lys Trp Leu Lys Glu Asp Lys
Leu Glu Cys Ser Glu Glu Leu Gly Asp Leu Val Lys Ser Val Asp Pro Thr Leu Ala Leu Ser Val
Tyr Leu Arg Ala Asn Val Pro Asn Lys Val Ile Gln Cys Phe Ala Glu Thr Gly Gln Val Gln Lys Ile
Val Leu Tyr Ala Lys Lys Val Gly Tyr Thr Pro Asp Trp Ile Phe Leu Leu Arg Asn Val Met Arg Ile
Ser Pro Asp Gln Gly Gln Gln Phe Ala Gln Met Leu Val Gln Asp Glu Glu Pro Leu Ala Asp Ile Thr
Gln Ile Val Asp Val Phe Met Glu Tyr Asn Leu Ile Gln Gln Cys Thr Ala Phe Leu Leu Asp Ala Leu
Lys Asn Asn Arg Pro Ser Glu Gly Pro Leu Gln Thr Arg Leu Leu Glu Met Asn Leu Met His Ala
Pro Gln Val Ala Asp Ala Ile Leu Gly Asn Gln Met Phe Thr His Tyr Asp Arg Ala His Ile Ala Gln
Leu Cys Glu Lys Ala Gly Leu Leu Gln Arg Ala Leu Glu His Phe Thr Asp Leu Tyr Asp Ile Lys
Arg Ala Val Val His Thr His Leu Leu Asn Pro Glu Trp Leu Val Asn Tyr Phe Gly Ser Leu Ser Val
Glu Asp Ser Leu Glu Cys Leu Arg Ala Met Leu Ser Ala Asn Ile Arg Gln Asn Leu Gln Ile Cys Val
Gln Val Ala Ser Lys Tyr His Glu Gln Leu Ser Thr Gln Ser Leu Ile Glu Leu Phe Glu Ser Lys Ser
Phe Glu Gly Leu Phe Tyr Phe Leu Gly Ser Ile Val Asn Phe Ser Gln Asp Pro Asp Val His Phe Lys
Tyr Ile Gln Ala Ala Cys Lys Thr Gly Gln Ile Lys Glu Val Glu Arg Ile Cys Arg Glu Ser Asn Cys
Tyr Asp Pro Glu Arg Val Lys Asn Phe Leu Lys Glu Ala Lys Leu Thr Asp Gln Leu Pro Leu Ile Ile
Val Cys Asp Arg Phe Asp Phe Val His Asp Leu Val Leu Tyr Leu Tyr Arg Asn Asn Leu Gln Lys
Tyr Ile Glu Ile Tyr Val Gln Lys Val Asn Pro Ser Arg Leu Pro Val Val Ile Gly Gly Leu Leu Asp
Val Asp Cys Ser Glu Asp Val Ile Lys Asn Leu Ile Leu Val Val Arg Gly Gln Phe Ser Thr Asp Glu
Leu Val Ala Glu Val Glu Lys Arg Asn Arg Leu Lys Leu Leu Leu Pro Trp Leu Glu Ala Arg Ile
His Glu Gly Cys Glu Glu Pro Ala Thr His Asn Ala Leu Ala Lys Ile Tyr Ile Asp Ser Asn Asn Asn
Pro Glu Arg Phe Leu Arg Glu Asn Pro Tyr Tyr Asp Ser Arg Val Val Gly Lys Tyr Cys Glu Lys
Arg Asp Pro His Leu Ala Cys Val Ala Tyr Glu Arg Gly Gln Cys Asp Leu Glu Leu Ile Asn Val
Cys Asn Glu Asn Ser Leu Phe Lys Ser Leu Ser Arg Tyr Leu Val Arg Arg Lys Asp Pro Glu Leu
Trp Gly Ser Val Leu Leu Glu Ser Asn Pro Tyr Arg Arg Pro Leu Ile Asp Gln Val Val Gln Thr Ala
Leu Ser Glu Thr Gln Asp Pro Glu Glu Val Ser Val Thr Val Lys Ala Phe Met Thr Ala Asp Leu
Pro Asn Glu Leu Ile Glu Leu Leu Glu Lys Ile Val Leu Asp Asn Ser Val Phe Ser Glu His Arg
Asn Leu Gln Asn Leu Leu Ile Leu Thr Ala Ile Lys Ala Asp Arg Thr Arg Val Met Glu Tyr Ile
Asn Arg Leu Asp Asn Tyr Asp Ala Pro Asp Ile Ala Asn Ile Ala Ile Ser Asn Glu Leu Phe Glu
Glu Ala Phe Ala Ile Phe Arg Lys Phe Asp Val Asn Thr Ser Ala Val Gln Val Leu Ile Glu His Ile
Gly Asn Leu Asp Arg Ala Tyr Glu Phe Ala Glu Arg Cys Asn Glu Pro Ala Val Trp Ser Gln Leu
Ala Lys Ala Gln Leu Gln Lys Gly Met Val Lys Glu Ala Ile Asp Ser Tyr Ile Lys Ala Asp Asp
Pro Ser Ser Tyr Met Glu Val Val Gln Ala Ala Asn Thr Ser Gly Asn Trp Glu Glu Leu Val Lys
Tyr Leu Gln Met Ala Arg Lys Lys Ala Arg Glu Ser Tyr Val Glu Thr Glu Leu Ile Phe Ala Leu
Ala Lys Thr Asn Arg Leu Ala Glu Leu Glu Glu Phe Ile Asn Gly Pro Asn Asn Ala His Ile Gln
Gln Val Gly Asp Arg Cys Tyr Asp Glu Lys Met Tyr Asp Ala Ala Lys Leu Leu Tyr Asn Asn
Val Ser Asn Phe Gly Arg Leu Ala Ser Thr Leu Val His Leu Gly Glu Tyr Gln Ala Ala Val Asp
Gly Ala Arg Lys Ala Asn Ser Thr Arg Thr Trp Lys Glu Val Cys Phe Ala Cys Val Asp Gly Lys
Glu Phe Arg Leu Ala Gln Met Cys Gly Leu His Ile Val Val His Ala Asp Glu Leu Glu Glu Leu
Ile Asn Tyr Tyr Gln Asp Arg Gly Tyr Phe Glu Glu Leu Ile Thr Met Leu Glu Ala Ala Leu Gly
Leu Glu Arg Ala His Met Gly Met Phe Thr Glu Leu Ala Ile Leu Tyr Ser Lys Phe Lys Pro Gln
Lys Met Arg Glu His Leu Glu Leu Phe Trp Ser Arg Val Asn Ile Pro Lys Val Leu Arg Ala Ala
Glu Gln Ala His Leu Trp Ala Glu Leu Val Phe Leu Tyr Asp Lys Tyr Glu Glu Tyr Asp Asn Ala
Ile Ile Thr Met Met Asn His Pro Thr Asp Ala Trp Lys Glu Gly Gln Phe Lys Asp Ile Ile Thr Lys
Val Ala Asn Val Glu Leu Tyr Tyr Arg Ala Ile Gln Phe Tyr Leu Glu Phe Lys Pro Leu Leu Leu
Asn Asp Leu Leu Met Val Leu Ser Pro Arg Leu Asp His Thr Arg Ala Val Asn Tyr Phe Ser Lys
Val Lys Gln Leu Pro Leu Val Lys Pro Tyr Leu Arg Ser Val Gln Asn His Asn Asn Lys Ser Val
Asn Glu Ser Leu Asn Asn Leu Phe Ile Thr Glu Glu Asp Tyr Gln Ala Leu Arg Thr Ser Ile Asp
Ala Tyr Asp Asn Phe Asp Asn Ile Ser Leu Ala Gln Arg Leu Glu Lys His Glu Leu Ile Glu Phe
Arg Arg Ile Ala Ala Tyr Leu Phe Lys Gly Asn Asn Arg Trp Lys Gln Ser Val Glu Leu Cys Lys
Lys Asp Ser Leu Tyr Lys Asp Ala Met Gln Tyr Ala Ser Glu Ser Lys Asp Thr Glu Leu Ala Glu
Glu Leu Leu Gln Trp Phe Leu Gln Glu Glu Lys Arg Glu Cys Phe Gly Ala Cys Leu Phe Thr Cys
Tyr Asp Leu Leu Arg Pro Asp Val Val Leu Glu Thr Ala Trp Arg His Asn Ile Met Asp Phe Ala
Met Pro Tyr Phe Ile Gln Val Met Lys Glu Tyr Leu Thr Lys Val Asp Lys Leu Asp Ala Ser Glu
Ser Leu Arg Lys Glu Glu Glu Gln Ala Thr Glu Thr Gln Pro Ile Val Tyr Gly Asn Leu Ser Leu
Leu Glu His His His His His His

In some embodiments, the clathrin heavy chain is a clathrin heavy chain having SEQ ID NO: 3:

(SEQ ID NO: 3)
Met Ala Gln Ile Leu Pro Ile Arg Phe Gln Glu His Leu Gln Leu Gln Asn Leu Gly Ile Asn
Pro Ala Asn Ile Gly Phe Ser Thr Leu Thr Met Glu Ser Asp Lys Phe Ile Cys Ile Arg Glu Lys Val
Gly Glu Gln Ala Gln Val Val Ile Ile Asp Met Asn Asp Pro Ser Asn Pro Ile Arg Arg Pro Ile Ser
Ala Asp Ser Ala Ile Met Asn Pro Ala Ser Lys Val Ile Phe Asn Ile Glu Met Lys Ser Lys Met Lys
Ala His Thr Met Thr Asp Asp Val Thr Phe Trp Lys Trp Ile Ser Leu Asn Thr Val Ala Leu Val Thr
Asp Asn Ala Val Tyr His Trp Ser Met Glu Gly Glu Ser Gln Pro Val Lys Met Phe Asp Arg His Ser
Ser Leu Ala Gly Cys Gln Ile Ile Asn Tyr Arg Thr Asp Ala Ala Leu Lys Ala Gly Lys Thr Leu Gln
Ile Lys Gln Lys Trp Leu Leu Leu Thr Gly Ile Ser Ala Gln Gln Asn Arg Val Val Gly Ala Met Gln
Leu Tyr Ser Val Asp Arg Lys Val Ser Gln Pro Ile Glu Gly His Ala Ala Ser Phe Ala Gln Phe Lys
Met Glu Gly Asn Ala Glu Glu Ser Thr Leu Phe Cys Phe Ala Val Arg Gly Gln Ala Gly Gly Lys
Leu His Ile Ile Glu Val Gly Thr Pro Pro Thr Gly Asn Gln Pro Phe Pro Lys Lys Ala Val Asp Val
Phe Phe Pro Pro Glu Ala Gln Asn Asp Phe Pro Val Ala Met Gln Ile Ser Glu Lys His Asp Val Val
Phe Leu Ile Thr Lys Tyr Gly Tyr Ile His Leu Tyr Asp Leu Glu Thr Gly Thr Cys Ile Tyr Met Asn
Arg Ile Ser Gly Glu Thr Ile Phe Val Thr Ala Pro His Glu Ala Thr Ala Gly Ile Ile Gly Val Asn Arg
Lys Gly Gln Val Leu Ser Val Cys Val Glu Glu Glu Asn Ile Ile Pro Tyr Ile Thr Asn Val Leu Gln
Asn Pro Asp Leu Ala Leu Arg Met Ala Val Arg Asn Asn Leu Ala Gly Ala Glu Glu Leu Phe Ala
Arg Lys Phe Asn Ala Leu Phe Ala Gln Gly Asn Tyr Ser Glu Ala Ala Lys Val Ala Ala Asn Ala
Pro Lys Gly Ile Leu Arg Thr Pro Asp Thr Ile Arg Arg Phe Gln Ser Val Pro Ala Gln Pro Gly Gln
Thr Ser Pro Leu Leu Gln Tyr Phe Gly Ile Leu Leu Asp Gln Gly Gln Leu Asn Lys Tyr Glu Ser Leu
Glu Leu Cys Arg Pro Val Leu Gln Gln Gly Arg Lys Gln Leu Leu Glu Lys Trp Leu Lys Glu Asp
Lys Leu Glu Cys Ser Glu Glu Leu Gly Asp Leu Val Lys Ser Val Asp Pro Thr Leu Ala Leu Ser
Val Tyr Leu Arg Ala Asn Val Pro Asn Lys Val Ile Gln Cys Phe Ala Glu Thr Gly Gln Val Gln Lys
Ile Val Leu Tyr Ala Lys Lys Val Gly Tyr Thr Pro Asp Trp Ile Phe Leu Leu Arg Asn Val Met Arg
Ile Ser Pro Asp Gln Gly Gln Gln Phe Ala Gln Met Leu Val Gln Asp Glu Glu Pro Leu Ala Asp Ile
Thr Gln Ile Val Asp Val Phe Met Glu Tyr Asn Leu Ile Gln Gln Cys Thr Ala Phe Leu Leu Asp Ala
Leu Lys Asn Asn Arg Pro Ser Glu Gly Pro Leu Gln Thr Arg Leu Leu Glu Met Asn Leu Met His
Ala Pro Gln Val Ala Asp Ala Ile Leu Gly Asn Gln Met Phe Thr His Tyr Asp Arg Ala His Ile Ala
Gln Leu Cys Glu Lys Ala Gly Leu Leu Gln Arg Ala Leu Glu His Phe Thr Asp Leu Tyr Asp Ile
Lys Arg Ala Val Val His Thr His Leu Leu Asn Pro Glu Trp Leu Val Asn Tyr Phe Gly Ser Leu Ser
Val Glu Asp Ser Leu Glu Cys Leu Arg Ala Met Leu Ser Ala Asn Ile Arg Gln Asn Leu Gln Ile Cys
Val Gln Val Ala Ser Lys Tyr His Glu Gln Leu Ser Thr Gln Ser Leu Ile Glu Leu Phe Glu Ser Lys
Ser Phe Glu Gly Leu Phe Tyr Phe Leu Gly Ser Ile Val Asn Phe Ser Gln Asp Pro Asp Val His Phe
Lys Tyr Ile Gln Ala Ala Cys Lys Thr Gly Gln Ile Lys Glu Val Glu Arg Ile Cys Arg Glu Ser Asn
Cys Tyr Asp Pro Glu Arg Val Lys Asn Phe Leu Lys Glu Ala Lys Leu Thr Asp Gln Leu Pro Leu
Ile Ile Val Cys Asp Arg Phe Asp Phe Val His Asp Leu Val Leu Tyr Leu Tyr Arg Asn Asn Leu
Gln Lys Tyr Ile Glu Ile Tyr Val Gln Lys Val Asn Pro Ser Arg Leu Pro Val Val Ile Gly Gly Leu
Leu Asp Val Asp Cys Ser Glu Asp Val Ile Lys Asn Leu Ile Leu Val Val Arg Gly Gln Phe Ser Thr
Asp Glu Leu Val Ala Glu Val Glu Lys Arg Asn Arg Leu Lys Leu Leu Leu Pro Trp Leu Glu Ala
Arg Ile His Glu Gly Cys Glu Glu Pro Ala Thr His Asn Ala Leu Ala Lys Ile Tyr Ile Asp Ser Asn
Asn Asn Pro Glu Arg Phe Leu Arg Glu Asn Pro Tyr Tyr Asp Ser Arg Val Val Gly Lys Tyr Cys
Glu Lys Arg Asp Pro His Leu Ala Cys Val Ala Tyr Glu Arg Gly Gln Cys Asp Leu Glu Leu Ile
Asn Val Cys Asn Glu Asn Ser Leu Phe Lys Ser Leu Ser Arg Tyr Leu Val Arg Arg Lys Asp Pro
Glu Leu Trp Gly Ser Val Leu Leu Glu Ser Asn Pro Tyr Arg Arg Pro Leu Ile Asp Gln Val Val Gln
Thr Ala Leu Ser Glu Thr Gln Asp Pro Glu Glu Val Ser Val Thr Val Lys Ala Phe Met Thr Ala Asp
Leu Pro Asn Glu Leu Ile Glu Leu Leu Glu Lys Ile Val Leu Asp Asn Ser Val Phe Ser Glu His
Arg Asn Leu Gln Asn Leu Leu Ile Leu Thr Ala Ile Lys Ala Asp Arg Thr Arg Val Met Glu Tyr
Ile Asn Arg Leu Asp Asn Tyr Asp Ala Pro Asp Ile Ala Asn Ile Ala Ile Ser Asn Glu Leu Phe
Glu Glu Ala Phe Ala Ile Phe Arg Lys Phe Asp Val Asn Thr Ser Ala Val Gln Val Leu Ile Glu
His Ile Gly Asn Leu Asp Arg Ala Tyr Glu Phe Ala Glu Arg Cys Asn Glu Pro Ala Val Trp Ser
Gln Leu Ala Lys Ala Gln Leu Gln Lys Gly Met Val Lys Glu Ala Ile Asp Ser Tyr Ile Lys Ala
Asp Asp Pro Ser Ser Tyr Met Glu Val Val Gln Ala Ala Asn Thr Ser Gly Asn Trp Glu Glu Leu
Val Lys Tyr Leu Gln Met Ala Arg Lys Lys Ala Arg Glu Ser Tyr Val Glu Thr Glu Leu Ile Phe
Ala Leu Ala Lys Thr Asn Arg Leu Ala Glu Leu Glu Glu Phe Ile Asn Gly Pro Asn Asn Ala His
Ile Gln Gln Val Gly Asp Arg Cys Tyr Asp Glu Lys Met Tyr Asp Ala Ala Lys Leu Leu Tyr Asn
Asn Val Ser Asn Phe Gly Arg Leu Ala Ser Thr Leu Val His Leu Gly Glu Tyr Gln Ala Ala Val
Asp Gly Ala Arg Lys Ala Asn Ser Thr Arg Thr Trp Lys Glu Val Cys Phe Ala Cys Val Asp Gly
Lys Glu Phe Arg Leu Ala Gln Met Cys Gly Leu His Ile Val Val His Ala Asp Glu Leu Glu Glu
Leu Ile Asn Tyr Tyr Gln Asp Arg Gly Tyr Phe Glu Glu Leu Ile Thr Met Leu Glu Ala Ala Leu
Gly Leu Glu Arg Ala His Met Gly Met Phe Thr Glu Leu Ala Ile Leu Tyr Ser Lys Phe Lys Pro
Gln Lys Met Arg Glu His Leu Glu Leu Phe Trp Ser Arg Val Asn Ile Pro Lys Val Leu Arg Ala
Ala Glu Gln Ala His Leu Trp Ala Glu Leu Val Phe Leu Tyr Asp Lys Tyr Glu Glu Tyr Asp Asn
Ala Ile Ile Thr Met Met Asn His Pro Thr Asp Ala Trp Lys Glu Gly Gln Phe Lys Asp Ile Ile Thr
Lys Val Ala Asn Val Glu Leu Tyr Tyr Arg Ala Ile Gln Phe Tyr Leu Glu Phe Lys Pro Leu Leu
Leu Asn Asp Leu Leu Met Val Leu Ser Pro Arg Leu Asp His Thr Arg Ala Val Asn Tyr Phe Ser
Lys Val Lys Gln Leu Pro Leu Val Lys Pro Tyr Leu Arg Ser Val Gln Asn His Asn Asn Lys Ser
Val Asn Glu Ser Leu Asn Asn Leu Phe Ile Thr Glu Glu Asp Tyr Gln Ala Leu Arg Thr Ser Ile
Asp Ala Tyr Asp Asn Phe Asp Asn Ile Ser Leu Ala Gln Arg Leu Glu Lys His Glu Leu Ile Glu
Phe Arg Arg Ile Ala Ala Tyr Leu Phe Lys Gly Asn Asn Arg Trp Lys Gln Ser Val Glu Leu Cys
Lys Lys Asp Ser Leu Tyr Lys Asp Ala Met Gln Tyr Ala Ser Glu Ser Lys Asp Thr Glu Leu Ala
Glu Glu Leu Leu Gln Trp Phe Leu Gln Glu Glu Lys Arg Glu Cys Phe Gly Ala Cys Leu Phe Thr
Cys Tyr Asp Leu Leu Arg Pro Asp Val Val Leu Glu Thr Ala Trp Arg His Asn Ile Met Asp Phe
Ala Met Pro Tyr Phe Ile Gln Val Met Lys Glu Tyr Leu Thr Lys Val Asp Lys Leu Asp Ala Ser
Glu Ser Leu Arg Lys Glu Glu Glu Gln Ala Thr Glu Thr Gln Pro Ile Val Tyr Gly Asn Leu Ser
Leu Leu Glu

In some embodiments, the clathrin light chain is a clathrin light chain having SEQ ID NO: 2:

(SEQ ID NO: 2)
Met Ala Glu Leu Asp Pro Phe Gly Ala Pro Ala Gly Ala Pro Gly Gly Pro Ala Leu Gly Asn
Gly Val Ala Gly Ala Gly Glu Glu Asp Pro Ala Ala Ala Phe Leu Ala Gln Gln Glu Ser Glu Ile Ala
Gly Ile Glu Asn Asp Glu Ala Phe Ala Ile Leu Asp Gly Gly Ala Pro Gly Pro Gln Pro His Gly Glu
Pro Pro Gly Gly Pro Asp Ala Val Asp Gly Val Met Asn Gly Glu Tyr Tyr Gln Glu Ser Asn Gly
Pro Thr Asp Ser Tyr Ala Ala Ile Ser Gln Val Asp Arg Leu Gln Ser Glu Pro Glu Ser Ile Arg Lys
Trp Arg Glu Glu Gln Met Glu Arg Leu Glu Ala Leu Asp Ala Asn Ser Arg Lys Gln Glu Ala Glu
Trp Lys Glu Lys Ala Ile Lys Glu Leu Glu Glu Trp Tyr Ala Arg Gln Asp Glu Gln Leu Gln Lys Thr
Lys Ala Asn Asn Arg Val Ala Asp Glu Ala Phe Tyr Lys Gln Pro Phe Ala Asp Val Ile Gly Tyr Val
Thr Asn Ile Asn His Pro Cys Tyr Ser Leu Glu Gln Ala Ala Glu Glu Ala Phe Val Asn Asp Ile Asp
Glu Ser Ser Pro Gly Thr Glu Trp Glu Arg Val Ala Arg Leu Cys Asp Phe Asn Pro Lys Ser Ser Lys
Gln Ala Lys Asp Val Ser Arg Met Arg Ser Val Leu Ile Ser Leu Lys Gln Ala Pro Leu Val His Leu
Glu His His His His His His

In some embodiments, the clathrin light chain is a clathrin light chain having SEQ ID NO: 4:

(SEQ ID NO: 4)
Met Ala Glu Leu Asp Pro Phe Gly Ala Pro Ala Gly Ala Pro Gly Gly Pro Ala Leu Gly Asn
Gly Val Ala Gly Ala Gly Glu Glu Asp Pro Ala Ala Ala Phe Leu Ala Gln Gln Glu Ser Glu Ile Ala
Gly Ile Glu Asn Asp Glu Ala Phe Ala Ile Leu Asp Gly Gly Ala Pro Gly Pro Gln Pro His Gly Glu
Pro Pro Gly Gly Pro Asp Ala Val Asp Gly Val Met Asn Gly Glu Tyr Tyr Gln Glu Ser Asn Gly
Pro Thr Asp Ser Tyr Ala Ala Ile Ser Gln Val Asp Arg Leu Gln Ser Glu Pro Glu Ser Ile Arg Lys
Trp Arg Glu Glu Gln Met Glu Arg Leu Glu Ala Leu Asp Ala Asn Ser Arg Lys Gln Glu Ala Glu
Trp Lys Glu Lys Ala Ile Lys Glu Leu Glu Glu Trp Tyr Ala Arg Gln Asp Glu Gln Leu Gln Lys Thr
Lys Ala Asn Asn Arg Val Ala Asp Glu Ala Phe Tyr Lys Gln Pro Phe Ala Asp Val Ile Gly Tyr Val
Thr Asn Ile Asn His Pro Cys Tyr Ser Leu Glu Gln Ala Ala Glu Glu Ala Phe Val Asn Asp Ile Asp
Glu Ser Ser Pro Gly Thr Glu Trp Glu Arg Val Ala Arg Leu Cys Asp Phe Asn Pro Lys Ser Ser Lys
Gln Ala Lys Asp Val Ser Arg Met Arg Ser Val Leu Ile Ser Leu Lys Gln Ala Pro Leu Val His Leu
Glu

In certain embodiments, the invention relates to a protein having a heavy chain, wherein the heavy chain has greater than 85% sequence homology to SEQ ID NO:1 or to SEQ ID NO: 3. In certain embodiments, the invention relates to any of the proteins described herein, wherein the heavy chain has greater than 90% sequence homology to SEQ ID NO:1 or to SEQ ID NO: 3. In certain embodiments, the invention relates to any of the proteins described herein, wherein the heavy chain has greater than 95% sequence homology to SEQ ID NO:1 or to SEQ ID NO: 3. In certain embodiments, the invention relates to any of the proteins described herein, wherein the heavy chain has greater than 98% sequence homology to SEQ ID NO:1 or to SEQ ID NO: 3. In certain embodiments, the invention relates to any of the proteins described herein, wherein the heavy chain has greater than 99% sequence homology to SEQ ID NO:1 or to SEQ ID NO: 3. In certain embodiments, the invention relates to any of the proteins described herein, wherein the heavy chain has SEQ ID NO:1 or SEQ ID NO: 3.

In certain embodiments, the invention relates to a protein having a light chain, wherein the light chain has greater than 85% sequence homology to SEQ ID NO:2 or to SEQ ID NO: 4. In certain embodiments, the invention relates to any of the proteins described herein, wherein the light chain has greater than 90% sequence homology to SEQ ID NO:2 or to SEQ ID NO: 4. In certain embodiments, the invention relates to any of the proteins described herein, wherein the light chain has greater than 95% sequence homology to SEQ ID NO:2 or to SEQ ID NO: 4. In certain embodiments, the invention relates to any of the proteins described herein, wherein the light chain has greater than 98% sequence homology to SEQ ID NO:2 or to SEQ ID NO: 4. In certain embodiments, the invention relates to any of the proteins described herein, wherein the light chain has greater than 99% sequence homology to SEQ ID NO:2 or to SEQ ID NO: 4. In certain embodiments, the invention relates to any of the proteins described herein, wherein the light chain has SEQ ID NO:2 or SEQ ID NO: 4.

In certain embodiments, the invention relates to any of the compositions described herein, wherein the heavy chain has a molecular weight from about 100 kDa to about 300 kDa. In certain embodiments, the invention relates to any of the compositions described herein, wherein the heavy chain has a molecular weight of about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 210 kDa, about 220 kDa, about 230 kDa, about 240 kDa, about 250 kDa, about 260 kDa, about 270 kDa, about 280 kDa, about 290 kDa, or about 300 kDa. In certain embodiments, the invention relates to any of the compositions described herein, wherein the heavy chain has a molecular weight of about 190 kDa.

In certain embodiments, the invention relates to any of the compositions described herein, wherein the light chain has a molecular weight from about 15 kDa to about 45 kDa. In certain embodiments, the invention relates to any of the compositions described herein, wherein the light chain has a molecular weight of about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, or about 45 kDa. In certain embodiments, the invention relates to any of the compositions described herein, wherein the light chain has a molecular weight of about 28 kDa.

In certain embodiments, the invention relates to any of the proteins described herein, wherein the protein has a heavy chain, a light chain, or a combination thereof.

In some embodiments, clathrin proteins are native. In other embodiments, clathrin proteins are truncated, elongated, mutated, or otherwise modified.

In some embodiments, scaffolding of truncated clathrin and their repeated sequences of these truncated peptides are used as payload carriers of anticancer internalizing peptides.

In some embodiments, clathrin cages sequester toxic chemo- and bio-therapeutic drug cargos while reducing toxic exposure to the whole body and increasing delivery exclusivity to imaging, marking, tumor/other disease, or other target sites. For example, in some embodiments, the clathrin cage complex is targeted to a tumor, cancer, or other neoplasm, where the complex is internalized by the tumor/cancer/neoplastic cells, where the environment triggers the capsule to release its toxic drug cargo.

In certain embodiments, the invention relates to any of the compositions described herein, wherein the three-dimensional cage structure has a diameter from about 10 nm to about 100 nm. In certain embodiments, the invention relates to any of the compositions described herein, wherein the three-dimensional cage structure has a diameter of about 10 nm, about 20 nm, about 30 nm, about 40 nm, about 50 nm, about 60 nm, about 70 nm, about 80 nm, about 90 nm, or about 100 nm. In certain embodiments, the invention relates to any of the compositions described herein, wherein the three-dimensional cage structures have an average diameter from about 10 nm to about 100 nm. In certain embodiments, the invention relates to any of the compositions described herein, wherein the three-dimensional cage structures have an average diameter of about 10 nm, about 20 nm, about 30 nm, about 40 nm, about 50 nm, about 60 nm, about 70 nm, about 80 nm, about 90 nm, or about 100 nm. In certain embodiments, the diameter of the three-dimensional cage structures may be estimated or measured by techniques known in the art, such as dynamic light scattering or high-resolution NMR spectroscopy.

In certain embodiments, the invention relates to any of the compositions described herein, wherein the three-dimensional cage structure is substantially spherical.

In certain embodiments, the invention relates to any of the compositions described herein, wherein the three-dimensional cage structure is non-covalently assembled, for example, self-assembled.

In certain embodiments, the invention relates to any of the compositions described herein, wherein the three-dimensional cage structure is substantially stable at about 37° C. at about pH greater than or equal to 7. In certain embodiments, the invention relates to any of the compositions described herein, wherein the three-dimensional cage structure is substantially stable at about 37° C. at about pH 7. In certain embodiments, the invention relates to any of the compositions described herein, wherein the three-dimensional cage structure is substantially stable at about 37° C. at about pH 6.5 to about pH 8. In certain embodiments, the invention relates to any of the compositions described herein, wherein the three-dimensional cage structure is substantially unstable at about 37° C. at about pH less than or equal to 5.5. In certain embodiments, the invention relates to any of the compositions described herein, wherein the three-dimensional cage structure is substantially unstable at about 37° C. at about pH 5.5.

Cage-like proteins such as clathrin, ferritins, DNA-binding proteins (dps), and heat shock proteins have three distinct surfaces (inside, outside, interface) that can be exploited to generate nanomaterials with multiple functionality by design. Protein cages are biological in origin and each cage exhibits extremely homogeneous size distribution. This homogeneity can be used to attain a high degree of homogeneity of the templated material and its associated property. A series of protein cages exhibiting diversity in size, functionality, and chemical and thermal stabilities can be utilized for materials synthesis under a variety of conditions. Because synthetic approaches to materials science often use harsh temperature and pH, in certain embodiments, it can be an advantage to utilize protein cages from extreme environments, such as acidic thermal hot springs.

Protein cage architectures, 10-100 nm in diameter, are self-assembled hollow spheres derived from viruses and other biological cages, including heat shock proteins (Hsp), DNA-binding proteins from starved cells (Dps), and ferritins. These architectures play critical biological roles. For example, heat shock proteins are thought to act as chaperones that prevent protein denaturation, and ferritins are known to store iron (which is both essential and toxic) as a nanoparticle of iron oxide. While each of these structures has evolved to perform a unique natural function, they are similar in that they are all essentially proteinaceous containers with three distinct surfaces (interior, exterior, and subunit interface) to which one can impart function by design. Protein cage architectures have demonstrated utility in nanotechnology with applications including inorganic nanoparticle synthesis and the development of targeted therapeutic and imaging delivery agents.

Protein cage architectures are naturally diverse; each has unique attributes (including size, structure, solvent accessibility, chemical and temperature stability, structural plasticity, assembly and disassembly parameters, and electrostatics) useful to particular applications. Importantly, one can capitalize on these features or alter them via genetic or chemical modification. Atomic level structural information identifies the precise location of amino acids within protein cage architectures and in turn allows for the rational inclusion, exclusion, and substitution of amino acid(s) (at the genetic level) resulting in protein cages with novel functional properties.

Protein cages isolated from thermophilic environments are desirable as building blocks for nanotechnology due to their potential stability in harsh reaction conditions including high temperature and pH extremes. Interestingly, one of the most stable protein cage architectures, ferritin, is commonly found in mesophilic organisms, including animals, plants, and microbes. For example, horse spleen ferritin exhibits broad pH (pH 2-8) and temperature stability (<70° C.). Ferritins are involved in iron sequestration, which they accomplish through the oxidation of soluble Fe(II) using O2. This oxidation results in the formation of a nanoparticle of Fe2O3 encapsulated (and rendered nontoxic) within the protein cage. High charge density on the inner surface of the protein cage promotes this reaction, which is assisted by an enzymatic (ferroxidase) activity in some ferritin subunits. Ferritins are made up of 24 subunits, which form a spherical cage 12 nm in diameter. The ferritin family also includes the 24 subunit bacterioferritins and the Dps class of proteins, which assemble from 12 monomers.

A cavity forming protein cage is described in U.S. Pat. No. 7,393,924 (incorporated by reference). The cage is formed in vitro from a plurality of 3-legged triskelia, each triskelion having 6 protein subunits; 3 clathrin heavy chain and 3 clathrin light chain subunits. In certain embodiments, the 3-legged triskelia are not required (see, e.g., U.S. Patent Application Publication No. 2015/0307570, incorporated by reference). For example, the protein may be an isolated, synthetic or recombinant, protein comprising in whole or in part one or more types of clathrin proteins of one or more isoforms, including cloned isoforms.

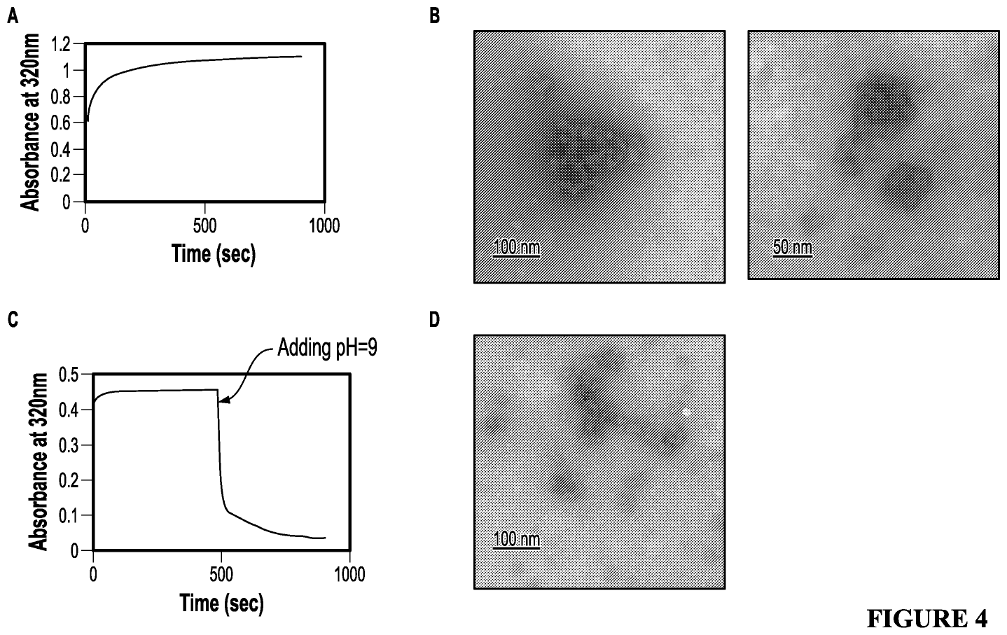

Protein cage architectures have three surfaces (interior, subunit interface, and exterior) amenable to both genetic and chemical modification. Each surface can play a distinct role in the development of new targeted therapeutic and imaging agent delivery systems. See FIG. 4B. The cage interior can house therapeutics, the subunit interface incorporates gadolinium (an MRI contrast agent) and the exterior presents cell-specific targeting ligands (such as peptides and antibodies).

Protein cages have many beneficial attributes that are useful in their development as targeted therapeutic and imaging agent delivery systems. Their size falls into the nanometer range shown to localize in tumors due to the enhanced permeability and retention effect. Their multivalent nature enables the incorporation of multiple functionalities (including targeting peptides and imaging agents) on a single protein cage. They are malleable to both chemical and genetic manipulation and can be produced in heterologous expression systems (including bacterial, yeast, and baculoviral systems). In addition, detailed atomic resolution structural information enables the rational design of genetic mutants with specific functions, including cell-specific targeting.

Another key component for the development of protein cage architectures as imaging and therapeutic agents is cell-specific targeting. In vivo application of the phage display library technique enabled the identification of peptides that bind specifically to the vasculature of particular organs as well as tumors. One of the most characterized of these targeting peptides is RGD-4C (CDCRGDCFC), which binds alphaVbeta3 and alphaVbeta5 integrins that are more prevalently expressed within tumor vasculature. For example, RGD-4C and other targeting peptides may be incorporated on the exteriors of the proteins. Fluorescein labeling of cell-specific targeted cages enables their visualization by epifluorescence microscopy. In addition to genetic incorporation, cell-specific targeting ligands, including antibodies and peptides, have also been chemically coupled to protein cage platforms. For example, an anti-CD4 monoclonal antibody conjugated to a protein could enable targeting of CD4+ lymphocytes within a population of splenocytes. The multivalent nature of protein cage architectures results in the presentation of multiple targeting ligands on their surfaces and may potentially aid in the interaction of these protein cages with many surfaces including receptors on a variety of cell types.

Chimeric Antigen Receptors

In some embodiments, the non-clathrin protein moiety comprises a chimeric antigen receptor (CAR). Generally, the CAR comprises an ectodomain, a transmembrane domain, and an endodomain.

When the CAR serves as a receptor on a cell surface, the ectodomain is the region of a receptor exposed to the outside of the cell, where its antigen-binding domain interacts with potential target molecules (e.g., potential antigens). In some embodiments, the CAR ectodomain comprises an antigen-binding domain. Examples of antigen-binding domains include, but are not limited to, domains that recognize and bind to a target cell of interest. Examples of a target cell of interest include, but are not limited to, a cell belonging to an infectious agent (e.g., a bacterial cell or parasite cell), a cell belonging to the subject but infected or otherwise compromised (e.g., by a bacterial cell, virus, or parasite, or by damage, such as DNA damage), a cell belonging to the subject but which expresses a particular cell-surface protein (e.g., a mutant cell, a diseased cell, a tumor cell, or a cancer cell). Other examples of a target cell of interest include, but are not limited to, a regulatory cell, a secretory cell (e.g., a hormone-secreting cell), a cell that promotes growth, mutagenesis, or metastasis of a tumor or other neoplasm (e.g., a growth hormone producing or secreting cell), a cell that inhibits or promotes cell death, or an immune effector cell or a cell that regulates an immune effector cell.

In some embodiments, the CAR is a first-generation CAR. In some embodiments, the CAR is a second-generation CAR. In some embodiments, the CAR is a third generation CAR. In some embodiments, the CAR is a fourth generation CAR (also known as an armored CAR or TRUCK). In some embodiments, the CAR is a UniCAR, a dual-antigen receptor CAR, or is part of an on-switch system.

A “first generation CAR” typically comprises, e.g., an antigen binding domain (e.g., a single chain variable fragment [scFv]), an extracellular hinge (optionally), a transmembrane domain (TMD), and an intracellular signaling domain (CD3-zeta [CD3ζ]). A “second generation CAR” typically includes, e.g., an additional costimulatory domain (e.g., CD28, CD137, CD3-zeta, or 4-1BB). A “third generation CAR” typically includes, e.g., multiple costimulatory domains (e.g., CD28, CD137, CD3-zeta, CD3-epsilon, 4-1BB, OX40). A “fourth generation CAR” (an “armored CAR” or a “TRUCK”) includes, e.g., an expression component (e.g., for expression of a cytokine [e.g., an interleukin, such as IL-2, IL-5, or IL-12; a costimulatory ligand; or an apoptosis or suicide inducer, such as caspase 9/inducible caspase 9 or HSV thymidine kinase]). The expression component is optionally inducible (e.g., iCasp9).

A “UniCAR” T cell recruitment system includes two components, namely, a universal CAR having an extracellular P1 (peptide or protein) domain attached to the hinge region and capable of binding to another peptide or protein P2, which is fused to an scFv recognizing a surface molecule on a target cell. “Dual-antigen receptor” CARs are engineered to express two antigen receptors at the same time (e.g., two tumor-associated antigens) to increase specificity of the T cells and to reduce side effects, including non-specific binding. In an “On-Switch” system, the CAR has a first receptor protein containing the antigen-binding domain and a second protein containing downstream signaling elements and costimulatory domains. The presence of an exogenous molecule causes dimerization of the binding and costimulatory proteins, e.g., to enable the CAR T cell to attack the tumor. Similarly, bispecific CAR molecules (e.g., CD20/CD3, fluorescein isothiocyanate [FITC]) can be used as switches, targeting a tumor-associated antigen and a surface molecule (e.g., CD3) on the surface of a T cell. In addition, a UniCAR T cell constructed to bind to a benign molecule (e.g., FITC) and coadministered with a bispecific small molecule drug conjugate (SMDC) adaptor molecule combining, e.g., a tumor-homing molecule with a FITC molecule with, e.g., anti-tumor activity induced only in the presence of both molecules.

“Antigens” (Ag) are structures or substances (e.g., proteins, polypeptides, polysaccharides) specifically bound by antibodies (Ab) (produced by a T cell) or by a B cell antigen receptor (BCR) (a surface receptor on a B cell), or in the case of CARs, are specifically bound by antigen-binding domains. With respect to CAR antigen-binding domains, examples include, but are not limited to, chimeric antigen receptor-polyclonal regulatory T cells (CAR-Treg cells) as immunosuppression regulatory cells that balance or regulate inflammation, as an example the neuroinflammation associated with neurodegenerative disease. CAR-T and B cells are part of the adaptive immune system. An antigen often includes multiple epitopes (i.e., distinct surface features of an antigen or antigenic determinant). Examples of antigens include, but are not limited to, interleukin10 (IL-10), transforming growth factor beta (TGF-beta, TGF-0), programmed cell death protein 1 (like PD-1), programmed death-ligand 1 (PD-L1; cluster of differentiation 274 [CD274] or B7 homolog 1 [1B7-H1]), cytotoxic T lymphocyte-associated protein 4 (CTLA-4), and in the case of CAR-Treg, T helper 17 (Th17), T helper 1 (Th1), cytotoxic T lymphocyte (CTL), M1 macrophage, and others.

In an CAR, the “antigen-binding site” or “antigen-binding domain” comprises the part of an CAR molecule comprised of the variable regions of an antigen-binding single-chain Fv (scFv) (e.g., a light chain [VL] and a heavy chain [VH], optionally linked by a scFv linker), such as an antigen-binding domain in a CAR.

Provided herein are chimeric protein constructs comprising a CAR having an ectodomain comprising at least one antigen-binding domain.

An antigen-binding domain or an affinity reagent binds to an antigen or receptor or other molecule. In some embodiments, an antigen-binding domain or an affinity reagent is a molecule that specifically binds to an antigen or receptor or other molecule. In certain embodiments, some or all of an antigen-binding domain or an affinity reagent is composed of amino acids (including natural, non-natural, and modified amino acids), nucleic acids, or saccharides. In certain embodiments, an affinity reagent or antigen-binding domain is a small molecule.

Antigen-binding domains in certain embodiments of the invention specifically bind to molecules or targets, such as a cell surface antigen, a cell surface receptor, or other cell surface molecule.

In some embodiments, the ectodomain comprises a scFv (e.g., a light chain and a heavy chain, optionally linked by a scFv linker) with an antigen binding domain. In some embodiments, the scFv has multiple light chains and/or multiple heavy chains (e.g., tandem scFvs, such as tandem di-scFv or tandem tri-scFv). Alternatively, the scFv has linker peptides that are too short for the two variable regions to fold together (e.g., approximately 5 amino acid residues) to yield a diabody or even shorter linker peptides (e.g., 1-2 amino acid residues) to yield a tri(a)body. In other embodiments, the variable fragments have specificity for two different antigens (i.e., two antigen-binding domains in a bispecific CAR). In some embodiments, the scFv is a dual-antigen receptor (e.g., expressing two tumor-associated antigen receptors simultaneously).

In certain embodiments, the invention relates to any of the compositions described herein, wherein the CAR comprises an anti-PD (program cell death proteins [e.g., anti-PD-1 or anti-PD-L1]) antigen-binding site.

Lymph nodes (LN) are a critical site of pathogenesis in immune-mediated diseases and cancer and, as major points of lymphocyte accumulation, are critical sites of targeting delivery of immunoregulatory molecules, checkpoint inhibitors, and chemotherapy drugs. For example, naïve T cells and central memory cells circulate between the blood and lymph nodes. L-selectin is expressed on leukocytes, L-selectin plays a key role in the continuous homing of naïve T cells to the LN. Further, it recognizes sulfated sialyl-LewisX-like sugars, called peripheral node addessin (PNAd), which are expressed by high endothelial venules (HEV) in the LN, attracting the naïve T-cells. Through this interaction, LN targeted delivery can markedly augment the therapeutic index of therapeutics, increasing their efficacy while reducing their toxicity.

The mesenteric lymph nodes (MLN; mesenteric glands) are one of the three principal groups of superior mesenteric lymph nodes, and approximately 100-150 MLN are located between layers of the mesentery in two main groups: the ileocolic group, which is situated near the wall of the small intestine, and the mesocolic group. The MLN are primarily located in the lower abdomen and lie throughout the various intestinal loops and near the superior mesenteric artery. They can often be affected by cancers in the abdominal region and by cancers that affect all lymph nodes (e.g., lymphoma).

Other types of lymph nodes include, but are not limited to, axillary, mediastinal, supratrochlear, and inguinal.

In some embodiments, the ectodomain further comprises a transport signal peptide (e.g., N-terminal to the scFv). In other embodiments, the ectodomain further comprises a spacer between the antigen binding domain and the transmembrane region, e.g., to serve as a flexible hinge region to provide flexibility in the orientation of the antigen-binding domain to make it more available for binding. In some embodiments, the spacer comprises a hinge domain from immunoglobulin G (IgG), a peptide or CD8.

When the CAR serves as a receptor on a cell surface, the transmembrane region is a structural component spanning the cell membrane and is often responsible for the stability of the receptor. In some embodiments, the transmembrane domain is largely hydrophobic (e.g., a hydrophobic alpha helix or beta barrel). In some embodiments, the transmembrane domain from the most membrane-proximal component of the endodomain is used. Alternatively, a different transmembrane domain is selected to provide a different receptor stability (e.g., an increased receptor stability). In one embodiment, the CD28 transmembrane domain is used.

When the CAR serves as a receptor on a cell surface, the endodomain is the region of the receptor that is the internal cytoplasmic end of the receptor that perpetuates signaling inside the T-cell. In some embodiments, the endodomain comprises one or more immunoreceptor tyrosine-based activation motifs (ITAMs), e.g., for T-cell activation via phosphorylation. In some embodiments, the endodomain comprises CD3-zeta (CD3ζ, CD247) or a cytoplasmic intracellular signaling domain thereof (including the CD3-zeta ITAM). Alternatively, one or more other ITAMs may be used or ITAMs from different sources.

In some embodiments, the endodomain further comprises at least one costimulatory domain, e.g., to aid T-cell activation. In some embodiments, the endodomain comprises cluster of differentiation 28 (CD28) or a costimulatory domain thereof; cluster of differentiation 137 (CD137; 4-1BB) or a costimulatory domain thereof; or cluster of differentiation 134 (CD134; OX40) or a costimulatory domain thereof; or cluster of differentiation 278 (CD278; inducible T cell costimulatory [ICOS]) or a costimulatory domain thereof. One or more costimulatory domains may be used, either identical or non-identical.

In some embodiments, the endodomain further comprises at least one nuclear factor of activated T-cell-responsive inducible expression element (resulting in a CAR that is a TRUCK or armored CAR). In some embodiments, the endodomain comprises a nuclear factor of activated T-cell-responsive inducible expression for an inducible transgenic cytokine, e.g., to enhance T cell expansion, persistence, and anti-tumor activity. Inducible transgenic cytokines include, but are not limited to, IL-2, IL-5, IL-12, and costimulatory ligands. In some embodiments, the nuclear factor of activated T-cell-responsive inducible element is NFAT5 (See www.ncbi.nlm.nih.gov/gene/10725; Gene ID: 10725, updated on 18 Nov. 2019; incorporated herein by reference).

In some embodiments, the CAR-expressing cell of interest (T-cells, B-cells, Treg cells) treats or alleviates a disease or an abnormal physiological condition. Examples of diseases and/or abnormal physiological conditions include, but are not limited to, a tumor, a cancer/other neoplasm, and neuroinflammation or a neurodegenerative disease (such as Alzheimer's Disease, Parkinson's Disease, amyotrophic lateral sclerosis [ALS], multiple sclerosis [MS], Progressive Supranuclear Palsy [PSP], or a prion disease).

In some embodiments, the term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

In some embodiments, the protein moiety or moieties of the present invention, including the CAR, is constructed, e.g., by using a nucleic acid vector encoding the protein, polypeptides, peptides, antigen-binding domains, and/or recombinant fusions provided herein. Modifications to the nucleic acid encoding the protein can be used to introduce a modification, truncation, or elongation of the expressed protein.

In one embodiment, provided herein are primers used for amplification and construction of the vectors and nucleic acids provided herein. It is to be understood by a skilled artisan that other primers can be used or designed to arrive at the vectors, nucleic acids and conjugates provided herein.

In one embodiment, provided herein is a vector comprising the nucleic acid encoding for the conjugate components provided herein. In another embodiment, the vector comprises nucleic acid encoding the protein, polypeptides, peptides, antigen-binding domains, and recombinant fusions provided herein. Modifications to the nucleic acid encoding the protein can be used to introduce a modification, truncation, or elongation of the expressed protein.

In some embodiments, the conjugates are purified or isolated after expression and prior to administration (e.g., CAR either before and/or after addition of the clathrin moiety; optional antibodies either before and/or after their addition to the chimeric protein construct). Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc, CARs, and other biomolecules, and these proteins can find use in the present invention for purification of conjugates. For example, the bacterial proteins A and G bind to the Fc region. Likewise, the bacterial protein L binds to the Fab region of some CARs, as of course does the CAR's target antigen. Purification can often be enabled by a particular fusion partner. For example, proteins may be purified using glutathione resin if a GST fusion is employed, Ni+2 affinity chromatography if a His-tag is employed, or immobilized anti-flag antibody, if a flag-tag is used. The degree of purification necessary will vary depending on the screen or use of the conjugates. In some instances, no purification is necessary. For example, in one embodiment, if the conjugates are secreted, screening may take place directly from the media. As is well known in the art, some methods of selection do not involve purification of proteins. Thus, for example, if a library of conjugates is made into a phage display library, protein purification may not be performed.

The CAR of interest may be produced via expression of a DNA construct, wherein the polynucleotides of the present invention are incorporated in a DNA construct enabling their expression in the organism's cell to produce a CAR of interest. DNA vector constructs suitable for use in subjects, tissues, cells, or cell lines are known to a person skilled in the art. According to one embodiment, the DNA construct comprises at least one expression regulating element selected from the group consisting of a promoter, an enhancer, an origin of replication, a transcription termination sequence, a polyadenylation signal and the like. The promoter can be constitutive, induced or tissue specific as is known in the art. Optionally, the DNA construct further comprises a selectable marker, enabling the convenient selection of the transformed cell/tissue. Additionally, or alternatively, a reporter gene can be incorporated into the construct, so as to enable selection of transformed cells or tissue expressing the reporter gene. The DNA constructs are designed according to the results to be achieved.

Payloads

In certain embodiments, the chimeric protein construct attached to clathrin construct further comprises a payload. In certain embodiments, the payload is conjugated, bound, linked, tethered, or fused to the clathrin protein moiety (a “clathrin-bound payload”) or to the CAR (e.g., in an manner to avoid blocking the antigen-binding domain) (a “CAR-bound payload”). In some embodiments, the clathrin protein moiety comprises a three-dimensional clathrin cage structure comprising an outer surface and an inner cavity, and the payload is conjugated, bound, linked, tethered, fused, or at least partially contained within the inner cavity of the clathrin cage structure. In some embodiments, the chimeric-clathrin protein composition further comprises an antibody or antigen-binding domain distinct from the antigen-binding domain of the CAR, and the payload is conjugated, bound, linked, tethered, or fused to the antibody or to the antigen-binding domain (e.g., in an manner to avoid blocking the antigen-binding domain of either the antibody or the antigen binding domain). Some embodiments comprise a combination of two or more of these approaches. In another non-limiting example, the chimeric protein construct comprises a clathrin cage or other clathrin moiety having a clathrin-bound payload and a CAR having a CAR-bound payload. In yet another non-limiting example, the chimeric protein construct comprises a clathrin cage or other clathrin moiety having a clathrin-bound payload, a CAR having a CAR-bound payload, and/or a payload attached to another component of the construct. In some embodiments two or more payloads are selected independently. In other embodiments, two or more payloads are identical. In some embodiments, two payloads are identical, while a third is selected independently.

In certain embodiments, the invention relates to any of the first compositions described herein, wherein the payload is any therapeutic agent, but preferably an anti-cancer agent, such as paclitaxel, protein-bound paclitaxel, docetaxel, gemcitabine, or an azonafide (e.g., a compound described in U.S. Pat. No. 8,008,316, which is incorporated by reference).

Pharmaceuticals and Other Therapeutic Agents

As used herein, the term “therapeutic agent” is defined broadly as anything that, e.g., organisms, organs, tissues, cells, antibodies, signal transduction factors, proteins, nucleic acids, bacteria, viruses, and the like, may be exposed to in a therapeutic protocol for the purpose of treating or curing a disease, disorder, or other aberrant biological condition. In some embodiments, it includes a preventive or prophylactic agent. In a non-limiting example, a therapeutic agent includes a drug or a pharmaceutical agent, composition, compound, drug or formulation. In one embodiment, such agents can be used according to the compositions and methods described herein in conjunction with each other, or in any combination thereof. As used herein, the term “drug” is defined broadly as any substance that causes a change in an organism's physiology or psychology when administered to the organism. As used herein, the terms “pharmaceutical drug,” “medicament,” “medication,” or “medicine,” are defined broadly as any chemical substance used to treat, cure, prevent, or diagnose a disease, disorder, or aberrant biological condition or to promote well-being. As used herein, the term “biologic,” “biologic drug,” “biologic response modifier,” or “biologic product” is defined broadly as any substance include a wide variety of products derived from human, animal, or microorganisms, e.g., by using biotechnology. In some embodiments, it may comprise, e.g., a protein or fragment of a protein that controls the action of (a) another protein or cellular process, (b) a gene that controls production of a protein of interest, (c) a modified human hormone, or (d) a cell that produces a substance that suppresses or activates a component of a biological pathway or system (e.g., the immune system).

As used herein, the terms “anti-cancer agent” and “anti-cancer therapeutic agent” are defined broadly as anything that cancer cells, including tumor cells, may be exposed to in a therapeutic protocol for the purpose of inhibiting their growth or kill the cells. In one embodiment, such agents can be used according to the compositions and methods described herein in conjunction with each other (e.g., LY294002 plus gemcitabine, TAXOL® (paclitaxel) plus U0126, TAXOL® plus gemcitabine, etc.), or in any combination thereof. Such agents include, but are not limited to, chemotherapeutic agents, such as anti-metabolic agents, e.g., Ara AC, 5-FU and methotrexate, antimitotic agents, e.g., TAXOL®, inblastine and vincristine, alkylating agents, e.g., melphalan, BCNU and nitrogen mustard, topoisomerase II inhibitors, e.g., VW-26, topotecan and Bleomycin, strand-breaking agents, e.g., doxorubicin and DHAD, cross-linking agents, e.g., cisplatin and CBDCA, radiation and ultraviolet light.

As used herein, the term “chemotherapeutic agent” is intended to include chemical reagents which inhibit the growth of proliferating cells or tissues wherein the growth of such cells or tissues is undesirable. Particular chemotherapeutic agents include, but are not limited to (i) antimetabolites, such as cytarabine, fludarabine, 5-fluoro-2′-deoxyuiridine, gemcitabine, hydroxyurea or methotrexate; (ii) DNA-fragmenting agents, such as bleomycin, (iii) DNA-crosslinking agents, such as chlorambucil, cisplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphtheria toxin; (vi) topoisomerase I poisons, such as camptothecin or topotecan; (vii) topoisomerase II poisons, such as etoposide (VP-16) or teniposide; (viii) microtubule-directed agents, such as colcemid, colchicine, paclitaxel, vinblastine or vincristine; (ix) kinase inhibitors such as flavopiridol, staurosporin, STI571 (CPG 57148B) or UCN-01 (7-hydroxystaurosporine); (x) enhancers of the AMPK signaling pathway, (xi) inhibitors of the PI3K/AKT/mTORC1 signaling pathway, (xii) inhibitors of the MEK/ERK signaling pathway, (xiii) miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; (xiv) hormones such as glucocorticoids or fenretinide; and (xv) hormone antagonists, such as tamoxifen, finasteride or LHRH antagonists. In some embodiments, the chemotherapeutic compound is one or more of gemcitabine, cisplatin, doxorubicin, daunarubicin, paclitaxel, protein-bound paclitaxel, docetaxel, taxotere and mitomycin C. In a particular embodiment, the chemotherapeutic compound is one or more of gemcitabine, cisplatin and paclitaxel.

Other chemotherapeutic agents, including biologics, are known in the art (see e.g., Gilman A. G., et al., The Pharmacological Basis of Therapeutics, 8th Ed., Sec 12:1202-1263 (1990)), and are typically used to treat neoplastic diseases. Chemotherapeutic agents generally employed in chemotherapy treatments are listed below in Table 1:

TABLE 1

| | | NONPROPRIETARY NAMES |
|---|---|---|
| CLASS | TYPE OF AGENT | (OTHER NAMES) |
| Alkylating | Nitrogen Mustards | Mec h foretham ine(HN₂) |
| | | Cyclophosphamide |
| | | Ifosfamide |
| | | Melphalan (L-sarcolysin) |
| | | Chlorambucil |
| | Ethylenimines | Hexamethy/melamine |
| | And Methyimelamines | Thiotepa |
| | Alkyl Sulfonates | Busulfan |
| | Nitrosoureas | Carustine (BCNU) |
| | | Lomustine (CCNU) |
| | | Semstine (methyl-CCNU) |
| | | Streptozocin (streptozotocin) |
| | Triazenes | Decarbazine (DTIC; imethyltriazenoimidazolecarboxamide) |
| | Alkylator | cis-diamminedichlorplatinum II (CDDP) |
| Antimetabolites | Folic Acid Analogs | Methotrexate (amethopterin) |
| | Pyrimidine Analogs | Fluorouracil (5-fluorouracil; 5-FU) |
| | | Flosuridine (forode-oxyuridine; FUdR) |
| | | Cytarabine (cytosine arabinoside) |
| | | gemcitabine (deoxycytidine analog) |
| | Purine Analogs and | Mercaptopaine (6-mercaptoparine; 6-MP) |
| | Related Inhibitors | Thiognsnine (6-thioguanine; TG) |
| | | Pentostatin (2′-deoxyeoformyein) |
| Natural Products | Vinca Alkaloids | Vinblastin (VLB) |
| | | Vincristine |
| | Topoisomerase inhibitors | Etoposide |
| | | Teniposide |
| | | Camptothecin |
| | | Topotecan |
| | | 9-amino-campotothecin CPT-11 |
| | Antibiotics | Dactinomycin (actinomycin D) |
| | | Adriamycin (Doxorubicin) |
| | | Daunorubicin (daunomycin; |
| | | rubindomycin) |
| | | Doxorubicin |
| | | Bleomycin |
| | | Plicamycin (mithramycin) |
| | | Mitomycin (mitomyein C) |
| | | TAXOL (paclitaxel) |
| | | Taxotere |
| | Enzymes | L-Asparaginase |
| | Biological Response | Interfon alfa |
| | Modifiers | interlenkin 2 |
| Misc. Agents | Platinum Coordination | cis-diamminedichloroplatinum II (CDDP) |
| | Complexes | Carboplat in |
| | | Oxaliplatin |
| | | Cisplatin |
| | Anthracendione | Mitoxantrone |
| | Substituted Urea | Hydroxyurea |
| | Methyl Hydraxzine | Procarbazine (N-methylbydrazine, |
| | Derivative | (MIE) |
| | Adrenocortical | Mitota ne (o.p′-DDD) |
| | Suppressant | Aminoglutethimide |
| Hormones and | Adrenocorticosteroids | Prednisone |
| Antagonists | Progestins | Dexamethasone |
| | | Hydroxyprogesterone |
| | | Caproate |
| | | Medroxyprogesterone |
| | | Acetate |
| | | Megestrol acetate |
| | Estrogens | Diethylstilbestrol |
| | | Ethinyi estradiol |
| | Antiestrogen | Tamoxifen |
| | Androgens | Testosterone propionate |
| | | Fluoxymesterone |
| | Antiandrogen | Flutamide |
| | Gonadotropin-releasing | Leuprolide |
| | Hormone analog | |

In certain embodiments, the chemotherapeutic agents used in the compositions and methods can be a single agent or a combination of agents. Preferred combinations will include agents that have different mechanisms of action, e.g., the use of an anti-mitotic agent in combination with an alkylating agent.

In some embodiments, the anti-cancer agent is an inhibitor of ERK signaling, such as an inhibitor of MEK. As used herein, the term “inhibitor of MEK” refers to a compound or agent, such as a small molecule, that inhibits, decreases, lowers, or reduces the activity of MEK. Examples of inhibitors of MEK include, but are not limited to, AZD6244 (6-(4-Bromo-2-chloro-phenylamino)-7-fluoro-3-methyl-3H-benzoimidazole-5-carboxylic acid (2-hydroxy-ethoxy)-amide; selumetinib; Structure IV), and U0126 (1,4-diamino-2,3-dicyano-1,4-bis [2-aminophenylthio]butadiene; ARRY-142886; Structure V). Further non-limiting examples of MEK inhibitors include PD0325901, AZD2171, GDC-0973/XL-518, PD98059, PD184352, GSK1120212, RDEA436, RDEA119/BAY869766, AS703026, BIX 02188, BIX 02189, CI-1040 (PD184352), PD0325901, and PD98059. These and other inhibitors of MEK, as well as non-limiting examples of their methods of manufacture, are described in U.S. Pat. Nos. 5,525,625; 6,251,943; 7,820,664; 6,809,106; 7,759,518; 7,485,643; 7,576,072; 7,923,456; 7,732,616; 7,271,178; 7,429,667; 6,649,640; 6,495,582; 7,001,905; US Patent Publication No. US2010/0331334, US2009/0143389, US2008/0280957, US2007/0049591, US2011/0118298, International Patent Application Publication No. WO98/43960, WO99/01421, WO99/01426, WO00/41505, WO00/42002, WO00/42003, WO00/41994, WO00/42022, WO00/42029, WO00/68201, WO01/68619, WO02/06213 and WO03/077914, the contents of which are herein incorporated by reference in their entireties.

In another embodiment, the anti-cancer agent is an inhibitor of epidermal growth factor receptor (EGFR). EGFR is a member of the type 1 subgroup of receptor tyrosine kinase family of growth factor receptors which play critical roles in cellular growth, differentiation and survival. Activation of these receptors typically occurs via specific ligand binding which results in hetero- or homodimerization between receptor family members, with subsequent autophosphorylation of the tyrosine kinase domain. Specific ligands which bind to EGFR include epidermal growth factor (EGF), transforming growth factor alpha (TGF alpha), amphiregulin and some viral growth factors. Activation of EGFR triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation. A number of human malignancies are associated with aberrant or overexpression of EGFR and/or overexpression of its specific ligands. Aberrant or overexpression of EGFR has been associated with an adverse prognosis in a number of human cancers, including head and neck, breast, colon, prostate, lung (e.g., NSCLC, adenocarcinoma and squamous lung cancer), ovarian, gastrointestinal cancers (gastric, colon, pancreatic), renal cell cancer, bladder cancer, glioma, gynecological carcinomas and prostate cancer. In some instances, overexpression of tumor EGFR has been correlated with both chemoresistance and a poor prognosis. Mutations in EGFR are associated with many types of cancer as well. For example, EGFR mutations are highly prevalent in non-mucinous BAC patients. Finberg, et al., J. Mol. Diagnostics. (2007) 9(3):320-26. In an embodiment the EGFR inhibitor is an antibody such as ERBITUTUX™ (cetuximab, Imclone Systems Inc.) and ABX-EGF (panitumumab, Abgenix, Inc.). In another embodiment the EGFR inhibitor is a small molecule that competes with ATP such as TARCEVA™ (erlotinib, OSI Pharmaceuticals), IRESSA™ (gefitinib, Astra-Zeneca), tyrphostins described by Dvir et al. (1991) J Cell Biol. 113:857-865; tricyclic pyrimidine compounds disclosed in U.S. Pat. No. 5,679,683; compound 6-(2,6-dichlorophenyl)-2-{[3-(hydroxymethyl)phenyl]amino}-8-methylpyrido[2,3-d]pyrimidin-7(8H)-one (or 6-(2,6-dichlorophenyl)-2-(4-(2-diethylaininoethoxy)phenylamino)-8-methyl-8H-pyrido(2,3-d)pyrimidin-7-one) (also known as PD166285) disclosed in Panek et al. (1997) Journal of Pharmacology and Experimental Therapeutics 283: 1433-1444.

Programmed cell death protein 1 (PD-1; cluster of differentiation 279 [CD279]) is a cell surface protein receptor and inhibitory checkpoint molecule that regulates the immune system's response to self by down-regulating the immune system and promoting self-tolerance by suppressing T cell inflammatory activity. While this activity prevents autoimmune diseases and rejection of a fetus or reduces the risk of a tissue graft or organ transplant, it also prevents the immune system from killing cancer cells. Programmed death-ligand 1 (PD-L1) is a transmembrane protein involved in suppressing adaptive immunity during pregnancy, tissue allografts, autoimmune disease, and certain other disease states (e.g., hepatitis). Binding of PD-L1 to PD-1 transmits an inhibitory signal that reduces the proliferation of antigen-specific T-cells in lymph nodes while reducing apoptosis in regulatory T cells (anti-inflammatory, suppressive T cells). PD-L1, the ligand of PD-1, is highly expressed in some cancers, resulting in the failure of the immune system to target cancer cells. This upregulation may allow cancers to evade the host immune system. These cancers can include, but are not limited to, renal cell carcinoma, melanoma, bladder cancer, gastric cancer, non-small cell lung cancer, lymphomas, mesothelioma, urothelial carcinoma, Merkel-cell carcinoma, head and neck cancers, and squamous cell carcinoma.

Inhibitors of PD-1 and/or PD-L1 include, but are not limited to, respectively, anti-PD-1 antibodies and/or anti-PD-L1 antibodies. Inhibitors of PD-1 include, but are not limited to, pembrolizumab (KEYTRUDA™; MK-3475; lambrolizumab; MERCK™), nivolumab (OPDIVO™; BRISTOL-MYERS SQUIBB™), cemiplimab (LIBTAYO™; REGENERON™), pidilizumab (CT-011; CURETECH™), and BMS-926559 (BRISTOL-MYERS SQUIBB™). Inhibitors of PD-L1 include, but are not limited to, atezolizumab (TECENTRIQ™; MPDL3280A; ROCHE™), avelumab (BAVENCIO™; MERCK™ [Germany] & PFIZER™), and durvalumab (IMFINZI™). Additional inhibitors of PD-1 include, but are not limited to, spartalizumab (PDR001; NOVARTIS™); camrelizumab (SHR1210; JIANGSU HENGRUIE™), sintilimab (IBI308; INNOVENT™ & ELI LILLY™), tislelizumab (BGB-A317), toripalimab (JS001), AMP-224 (GLAXOSMITHKLINE™), AMP-514 (GLAXOSMITHKLINE™), and nivolumab (BMS-936558; BRISTOL-MYERS SQUIBB™). Additional inhibitors of PD-L1 include, but are not limited to, KN035, CK-301 (CHECKPOINT THERAPEUTICS™), AUNP12 (AURIGENE™ & LABORATOIRES PIERRE FABRE™), CA-170 (AURIGENE™/CURIS™), and BMS-986189 (BRISTOL-MYERS SQUIBB™).

Another protein, on T cells, that inhibits the immune system is CTLA-4. Ipilimumab (YERVOY™), a monoclonal antibody that inhibits CTLA-4, has been used to treat melanoma and other cancers.

In certain embodiments, the invention relates to any of the compositions described herein, wherein the payload comprises an anti-PD-1 antibody or an anti-PD-1-antigen-binding domain. In certain embodiments, the invention relates to any of the composition described herein, wherein the payload comprises an anti-PD-1 antibody or an anti-PD-1 antigen-binding domain in combination with either (a) an anti-PD-L1 antibody or an anti-PD-L1 antigen-binding domain or (b) an anti-CTLA-4 antibody or an anti-CTLA-4 antigen-binding domain.

Nucleic Acids, Antisense Molecules, and RNA Interference (RNAi) Molecules

In addition to the specific agents described above, it is further contemplated that a polypeptide, an antibody or antigen binding fragment thereof, a toxin, an RNA interfering molecule, an siRNA molecule, and shRNA molecule, an antisense oligonucleotide, a peptide, a peptidomimetic, an aptamer, and the like, as well as combinations thereof, that appropriately enhance or inhibit the targets of pro-survival signaling pathways can also be used as a therapeutic agent according to the invention. In particular, the nucleic acid sequence, amino acid sequence, functional domain, structural domain, gene locus, and other identifying information for the signaling pathway targets described herein are well known in the art.

In certain embodiments, the payload is an siRNA moiety comprised of a sense strand and an antisense strand; the sense strand comprising a 3′ end and a 5′ end; and the antisense strand comprising a 3′ end and a 5′ end.

“Antisense” nucleic acids refer to nucleic acids that specifically hybridize (e.g., bind) with a complementary sense nucleic acid, e.g., cellular mRNA and/or genomic DNA, under cellular conditions so as to inhibit expression (e.g., by inhibiting transcription and/or translation). The binding may be by conventional base pair complementarity or, for example, in the case of binding to DNA duplexes, through specific interactions in the major groove of the double helix.

Antisense technology is the process in which an antisense RNA or DNA molecule interacts with a target sense DNA or RNA strand. A sense strand is a 5′ to 3′ mRNA molecule or DNA molecule. The complementary strand, or mirror strand, to the sense is called an antisense. When an antisense strand interacts with a sense mRNA strand, the double helix is recognized as foreign to the cell and will be degraded, resulting in reduced or absent protein production. Although DNA is already a double stranded molecule, antisense technology can be applied to it, building a triplex formation.

One skilled in the art would appreciate that the terms “complementary” or “complement thereof” are used herein to encompass the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

RNA antisense strands can be either catalytic or non-catalytic. The catalytic antisense strands, also called ribozymes, cleave the RNA molecule at specific sequences. A non-catalytic RNA antisense strand blocks further RNA processing.

Antisense modulation of cells and/or tissue levels of the globulin genes of interest and/or desaturase genes of interest or any combination thereof may be effected by transforming the organism's cells or tissues with at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog.

Antisense modulation of cells and/or tissue levels of the globulin genes of interest and/or desaturase genes of interest or any combination thereof may be effected by transforming the organism's cells or tissues with at least one antisense compound, including antisense DNA, antisense RNA, a ribozyme, DNAzyme, a locked nucleic acid (LNA), and an aptamer. In some embodiments, the molecules are chemically modified. In other embodiments, the antisense molecule is antisense DNA or an antisense DNA analog.

RNAi refers to the introduction of homologous double stranded RNA (dsRNA) to target a specific gene product, resulting in post transcriptional silencing of that gene. This phenomenon was first reported in Caenorhabditis elegans by Guo and Kemphues (1995, Cell, 81(4):611-620) and subsequently Fire et al. (1998, Nature 391:806-811) discovered that it is the presence of dsRNA, formed from the annealing of sense and antisense strands present in the in vitro RNA preps, that is responsible for producing the interfering activity

In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available from commercial sources.

The dsRNA can be transcribed from the vectors as two separate strands. In other embodiments, the two strands of DNA used to form the dsRNA may belong to the same or two different duplexes in which they each form with a DNA strand of at least partially complementary sequence. When the dsRNA is thus produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

One skilled in the art would appreciate that the terms “promoter element,” “promoter,” or “promoter sequence” may encompass a DNA sequence that is located at the 5′ end (i.e. precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full-length of the gene or more.

The term “RNA interference” or “RNAi” refers to the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

One of ordinary skill in the art would appreciate that the term RNAi molecule refers to single- or double-stranded RNA molecules comprising both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available as exemplified herein below.

The dsRNA can be transcribed from the vectors as two separate strands. In other embodiments, the two strands of DNA used to form the dsRNA may belong to the same or two different duplexes in which they each form with a DNA strand of at least partially complementary sequence. When the dsRNA is thus produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full-length of the gene or more.

In some embodiments, the payload is a small interfering RNA (siRNA). The siRNA moiety may further include a guanosine at the 5′-end.

The sense and/or antisense strands of the siRNA moiety may equal to or less than 30, 25, 24, 23, 22, 21, 20, 19, 18 or 17 nucleotides in length. An siRNA moiety may include one or more overhangs. For example, the siRNA moiety may include one or two 3′ overhangs of 2-3 nucleotides. In certain embodiments, the invention relates to any of the compositions described herein, wherein the siRNA moiety is composed of 21-nt sense and 21-nt antisense strands, paired in a manner to have a 19-nucleotide duplex region and a 2-nt 3′ overhang at each 3′ terminus. In certain embodiments, the invention relates to any of the compositions describe herein, wherein the 2-nt 3′ overhang is either UU or dTdT. Symmetric 3′-overhangs ensure that the sequence-specific endonuclease complexes (siRNPs) are formed with approximately equal ratios of sense and antisense target RNA cleaving siRNPs. The 3′-overhang in the sense strand provides no contribution to recognition as it is believed the antisense siRNA strand guides target recognition. Therefore, the UU or dTdT 3′-overhang of the antisense sequences is complementary to the target mRNA but the symmetrical UU or dTdT 3′-overhang of the sense siRNA oligo does not need to correspond to the mRNA. The use of deoxythymidines in both 3′-overhangs may increase nuclease resistance, although siRNA duplexes with either UU or dTdT overhangs work equally well. 2′-Deoxynucleotides in the 3′ overhangs are as efficient as ribonucleotides, but are often cheaper to synthesize.

The targeted region in the mRNA, and hence the sequence in the siRNA duplex, are chosen using the following guidelines. The open reading frame (ORF) region from the cDNA sequence is recommended for targeting, preferably at least 50 to 100 nucleotides downstream of the start codon, most preferably at least 75-100. Both the 5′ and 3′ untranslated regions (UTRs) and regions near the start codon are not recommended for targeting as these may be richer in regulatory protein binding sites. UTR-binding proteins and/or translation initiation complexes may interfere with binding of the siRNP endonuclease complex.

The sequence of the mRNA or cDNA is searched seeking the sequence AA(N19)TT. Sequences with approximately 50% G/C-content (30% to 70%) are used. If no suitable sequences are found, the search is extended to sequences AA(N21). The sequence of the sense siRNA corresponds to 5′-(N19)dTdT-3′ or N21, respectively. In the latter case, the 3′ end of the sense siRNA is converted to dTdT. The rationale for this sequence conversion is to generate a symmetric duplex with respect to the sequence composition of the sense and antisense 3′ overhangs. It is believed that symmetric 3′ overhangs help to ensure that the siRNPs are formed with approximately equal ratios of sense and antisense target RNA-cleaving siRNPs. The modification of the overhang of the sense sequence of the siRNA duplex is not expected to affect targeted mRNA recognition, as the antisense siRNA strand glides target recognition.

If the target mRNA does not contain a suitable AA(N21) sequence, it is recommended to search for NA(N21) The sequence of the sense and antisense strand may still be synthesized as 5′ (N19)TT as the sequence of the 3′ most nucleotide of the antisense siRNA does not appear to contribute to specificity.

It is further recommended to search the selected siRNA sequence against EST libraries in appropriate databases (e.g., NCBI BLAST database search) to ensure that only one gene is targeted.

The appropriately designed siRNAs are either obtained from commercial sources (such as Dharmacon Research, Lafayette, Colo.; Xergon, Huntsville, Ala.; Ambion, Austin, Tex.) or chemically synthesized used appropriately protected ribonucleoside phosphoramidites and a conventional DNA/RNA synthesizer according to standard protocols. The RNA oligonucleotides are 2′-deprotected, desalted and the two strands annealed, according to manufacturer's specifications or conventional protocols, depending on how the siRNAs are obtained. All handling steps are conducted under strict sterile, RNase-free conditions.

In some embodiments, a nucleic acid aptamer is included. Nucleic acid aptamers are nucleic acid oligomers that bind other macromolecules specifically; such aptamers that bind specifically to other macromolecules can be readily isolated from libraries of such oligomers by technologies such as SELEX. In some embodiments, an oligosaccharide is included. Certain oligosaccharides are known ligands for certain extracellular or cell surface receptors.

Co-Suppression Molecules

In some embodiments, the payload is a co-suppression molecule. A co-suppression molecule is another agent capable of down-regulating the expression of a given gene, or a combination thereof. Co-suppression is a post-transcriptional mechanism where both the transgene and the endogenous gene are silenced.

Enzymatic Nucleic Acid Molecules

In some embodiments, the payload is an enzymatic nucleic acid molecule. The terms “enzymatic nucleic acid molecule” or “enzymatic oligonucleotide” refers to a nucleic acid molecule which has complementarity in a substrate binding region to a specified gene target and also has an enzymatic activity which is active to specifically cleave target RNA of a given gene, thereby silencing each of the genes. The complementary regions allow sufficient hybridization of the enzymatic nucleic acid molecule to the target RNA and subsequent cleavage. The term enzymatic nucleic acid is used interchangeably with for example, ribozymes, catalytic RNA, enzymatic RNA, catalytic DNA, aptazyme or aptamer-binding ribozyme, catalytic oligonucleotide, nucleozyme, DNAzyme, RNAenzyme. The specific enzymatic nucleic acid molecules described in the instant application are not limiting and an enzymatic nucleic acid molecule of this invention requires a specific substrate binding site which is complementary to one or more of the target nucleic acid regions, and that it have nucleotide sequences within or surrounding that substrate binding site which impart a nucleic acid cleaving and/or ligation activity to the molecule. U.S. Pat. No. 4,987,071 discloses examples of such molecules.

In some embodiments, the payload is a DNAzyme molecule. A DNAzyme molecule is another agent capable of down-regulating the expression of a given gene, the DNAzyme molecule being capable of specifically cleaving an mRNA transcript or a DNA sequence of said gene. DNAzymes are single-stranded polynucleotides that are capable of cleaving both single- and double-stranded target sequences. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (for review of DNAzymes, see: Khachigian, L. M. (2002) Curr Opin Mol Ther 4, 119-121).

Examples of construction and amplification of synthetic, engineered DNAzymes recognizing single- and double-stranded target cleavage sites are disclosed in U.S. Pat. No. 6,326,174.

Imaging Agents, Diagnostic Agents, and Biomarkers

In certain embodiments, the payload is an imaging agent or a diagnostic agent or a biomarker.

For example, the imaging agent may be a fluorescent imaging agent, such as a fluorophore or a radio-gadolinium chelator, a radiofluorinated or radioiodinated radiolabeled agent, or a magnetic imaging agent, such as a magnetite mineral, a paramagnetic metal ion, or a metal chelating peptide. The imaging agent may be bound to an endogenous site (e.g., a paramagnetic metal ion), bound to a chemically modified site (e.g., chemical modifications to covalently bind a fluorophore, radiolabeled or a chelator), or genetically incorporated (e.g., a metal chelating peptide).

Examples of imaging or diagnostic agents include fluorophores (e.g. Dy547), chromophores, chemoluminescing agents, radionuclides (e.g., In-111, Tc-99m, I-123, I-125 F-18, Ga-67, Ga-68) for Positron Emission Tomography (PET) and Single Photon Emission Tomography (SPECT), unpair spin atoms and free radicals (e.g., Fe, lanthanides, and Gd), and contrast agents (e.g., chelated (DTPA) manganese) for Magnetic Resonance Imaging (MRI). Additional examples include, but are not limited to, IRDye 800CW (IR800 or IR-800), ALEXA FLUOR® 594 (THERMOFISHER™), CF680, and green fluorescent protein (GFP). 4′,6-diamidino-2-phenylindole (DAPI; IUPAC 2-(4-amidinophenyl)-1H-indole-6-carboxamidine), a fluorescent stain, binds strongly to adenine-thymine rich regions in DNA. Many imaging or diagnostic agents are commercially available.

Additional examples include radionuclides (e.g. C-11, F-18, 1-124, 1-123, 1-125, 1-131, Re-186, Re-188, Y-90, Bi-212, At-211, Sr-89, Ho-166, Sm-153, Cu-67, Cu-64, In-111, Tc-99m, Ga-67, and Ga-68).

A skilled artisan would appreciate that the term “biomarker” comprises any measurable substance in a cell, cell line, tissue or organism whose presence is indicative of a biological state or a condition of interest. In some embodiments, the presence of a biomarker is indicative of the presence of a compound or a group of compounds of interest. In some embodiments, the concentration of a biomarker is indicative of the concentration of a compound or a group of compounds of interest. In some embodiments, the concentration of a biomarker is indicative of a cell, cell line, tissue, or organism phenotype.

In a non-limiting example, podoplanin (PDPN) is a mucin-type protein that is well-conserved between species and is generally receptive to detection via immunofluorescent staining. It is a specific lymphatic vessel marker. Alternatively, spliced transcript variants have been identified. PDPN has functions in a wide range of cells, including lung alveolar cells, kidney podocytes, and lymphatic endothelial cells, as well as having been observed in human and murine neural tissue. Because its upregulation correlates with poor patient prognosis, PDPN can be used as a diagnostic marker for some types of cancers, including, but not limited to, various squamous cell carcinomas, malignant mesothelioma, and brain tumors. It is believed to play a key role in invasiveness of squamous cell carcinomas.

In another non-limiting example, in addition to being a fluorescent marker, 4′,6-diamidino-2-phenylindole (DAPI; IUPAC 2-(4-amidinophenyl)-1H-indole-6-carboxamidine), a fluorescent stain, binds strongly to adenine-thymine rich regions in DNA.

Antibodies, Antigen-Binding Sites, and Other Immunogens

In certain embodiments, the payload is an immunogen, such as an antibody or other antigen-binding site moiety or an affinity reagent. An “antibody,” an “antigen-binding site” or an “affinity reagent,” is a molecule that binds to an antigen or receptor or another molecule. In some embodiments, an antigen-binding site is a molecule that specifically binds to an antigen or receptor or other molecule. In certain embodiments, some or all of the immunogen is composed of amino acids (including natural, non-natural, and modified amino acids), nucleic acids, or saccharides. In certain embodiments, an antibody-binding site or affinity reagent or other immunogens a small molecule. In certain embodiments, antibodies specifically bind to molecules or targets, such as a cell surface antigen, a cell surface receptor, or other cell surface molecule. Antibodies are discussed in more detail, infra.

In certain embodiments, the payload is an immunogen, for example, an immunogenic antigen. An immunogen is an antigen or any substance that may be specifically bound by components of the immune system (e.g., antibody, lymphocytes). An immunogen is capable of inducing humoral or cell-mediated immune response rather than immunological tolerance. For example, the immunogen may be selected from the group consisting of keyhole limpet hemocyanin (KLH), concholepas concholepas hemacyanin (CCH), bovine serum albumin (BSA), and ovalbumin (OVA). Further information may be found in Chen et al. (2013) Immunity 39:1-10; and Chen et al. (2012) Clin Cancer Res. 18:6580-6587 (both incorporated by reference).

In some embodiments, the composition comprises an antibody (IgG or IgM based or their truncated forms). In some embodiments the antibody is attached to the clathrin (Protin-101) with or without a payload; in some embodiments, the antibody is attached to the CAR. In some embodiments, it is attached to another element in the construct directly. In some embodiments, it is attached to another element in the construct via a linker. In some embodiments, the antibody targets a cancer or other tumor cell. Essentially, the antibody specifically targets a tumor antigen of interest on a tumor cell of interest. The antibody binds to the antigen on the surface of the tumor cell, triggering a signal in the tumor cell, which then absorbs or internalizes the antibody along with the linked CAR. The specific targeting of the cancer cell reduces side effects. Some embodiments include targeting by Protin101-CAR and a non-CAR antibody providing even more specific discrimination of the target cell. Antibody linkers include, but are not limited to disulfides, hydrazones, peptides, or thioethers. In some embodiments, they are cleavable (e.g., peptides), while in other embodiments, they are non-cleavable (e.g., thioethers). Cleavable linkers may be engineered to be enzyme-sensitive. Non-cleavable linkers typically offer increased stability and maintain the drug within the cell. Longer linkers provide greater physical flexibility in the linker region, potentially altering cleavage kinetics.

In some embodiments, the composition comprises a Protin-101-antibody-drug conjugate (ADC). An “antibody-drug conjugate” comprises a Protin-101-antibody and a drug payload, optionally joined by an “ADC linker.” In some embodiments, the ADC comprises a Protin-101-antibody that targets a cancer cell and the drug payload comprises a cytotoxic drug that destroys the cancer cell. This type of bioconjugate/immunoconjugate combines the targeting capability of a monoclonal antibody with the cancer cell-destroying ability of a cytotoxic drug. Essentially, the antibody specifically targets a tumor antigen of interest on a tumor cell of interest. The antibody-Protin-101-Payload binds to the antigen on the surface of the tumor cell, triggering a signal in the tumor cell, which then absorbs or internalizes the antibody-Protin-101-payload along with the linked cytotoxin, which in turn, kills the cancer cell. Some embodiments include targeting by both the CAR-T(B)-cells and/or CAR-Treg attached to a non-CAR antibody providing even more specific discrimination of the target cell. ADC linkers include, but are not limited to disulfides, hydrazones, peptides, or thioethers. In some embodiments, they are cleavable (e.g., peptides), while in other embodiments, they are non-cleavable (e.g., thioethers). Cleavable ADC linkers may be engineered to be enzyme-sensitive. Non-cleavable ADC linkers typically offer increased stability and maintain the drug within the cell. Longer ADC linkers provide greater physical flexibility in the ADC linker region, potentially altering cleavage kinetics.

In some embodiments, the Protin-101 attached payload comprises programmed cell death protein 1 (PD-1; cluster of differentiation 279 [CD279]), a cell surface protein, a member of the immunoglobulin superfamily, is expressed on T cells and pro-B cells and promotes self-tolerance by suppressing T-cell inflammatory activity, preventing autoimmune diseases, but also inhibiting the immune system from killing cancer cells. Programmed cell death protein 1 (PD-1; cluster of differentiation 279 [CD279]), is a cell surface protein that has a role in regulating the immune system's response to the cells of the human body by down-regulating the immune system and promoting self-tolerance by suppressing T cell inflammatory activity. An immune checkpoint protein, PD-1 promotes apoptosis of antigen-specific T-cells in lymph nodes and reduces apoptosis in regulatory T-cells (Tregs). PD-1 is a cell surface receptor that belongs to the immunoglobulin superfamily and is expressed on T cells and pro-B cells. PD-1 binds two ligands, programmed death-ligand 1 (PD-L1) and programmed death-ligand 2 (PD-L2). PD-L1 is highly expressed on the surface of cells of some types of cancers, including, but not limited to, melanoma, bladder cancer, and gastric cancer. As a result, PD-1 inhibitors block PD-1 and lower immune system activation when attacking tumors.

In certain embodiments, the invention relates to any of the compositions described herein, wherein the payload comprises an anti-PD-1 antibody or an anti-PD-1-antigen-binding domain.

Cytotoxic T-lymphocyte-associated protein 4 (CTLA4 or CTLA-4; CD152 [cluster of differentiation 152]) is a protein receptor that functions as an immune checkpoint and downregulates immune responses. CTLA-4 is constitutively expressed in regulatory T cells, but only upregulated in conventional T cells after activation. This situation is particularly observed in some cancers. For example, it can serve as an “off” switch when bound to CD80 or CD86 on the surface of antigen-presenting cells.

In certain embodiments, the invention relates to any of the composition described herein, wherein the payload comprises an anti-PD-1 antibody or an anti-PD-1 antigen-binding domain in combination with either (a) an anti-PD-L1 antibody or an anti-PD-L1 antigen-binding domain or (b) an anti-CTLA-4 antibody or an anti-CTLA-4 antigen-binding domain.

Lymph nodes (LN) are a critical cite of pathogenesis in immune-mediated diseases and cancer and are critical sites of targeting delivery of immunoregulatory molecules, check point inhibitors, and chemotherapy drugs. LN targeted delivery can markedly augment the therapeutic index of therapeutics, increasing their efficacy while reducing their toxicity. In certain embodiments, the construct relates to any of the compositions described herein, wherein the targeting composition is an anti-peripheral lymph node addressin (PNAd) or wherein the antigen-binding site is an anti-PNAd antigen-binding site.

In some embodiments, an antigen-binding domain may be comprised of proteinaceous structures other than antibodies that are able to bind to protein targets specifically, including but not limited to avimers, ankyrin repeats and adnectins, and other such proteins with domains that can be evolved to generate specific affinity for antigens, collectively referred to as “antibody-like molecules.” Modifications of proteinaceous affinity reagents through the incorporation of unnatural amino acids during synthesis may be used to improve their properties. Such modifications may have several benefits, including the addition of chemical groups that facilitate subsequent conjugation reactions. In some embodiments, the antigen-binding domain may be a peptide. In some embodiments, the peptide chain is a bispecific peptide. Peptides can readily be made and screened to create affinity reagents that recognize and bind to macromolecules such as proteins.

Bispecific affinity reagents may be constructed by separate synthesis and expression of the first and second affinity reagents. A polypeptide bispecific reagent can be expressed as two separately encoded chains that are linked by disulfide bonds during production in the same host cell, such as, for example, a bispecific scFv or diabody. Similarly, standard and widely used solid-phase peptide synthesis technology can be used to synthesize peptides, and chimeric bispecific peptides are well known in the art. A bispecific peptide strategy may be used to combine the first and second first and second affinity reagents in a single peptide chain. Alternatively, polypeptide chains or peptide chains can be expressed/synthesized separately, purified and then conjugated chemically to produce the bispecific affinity reagents useful in the compositions and methods described herein. Many different formats of antibodies may be used. Whole antibodies, F(ab′)2, F(ab′), scFv, as well as smaller Fab and single-domain antibody fragments may all be used to create the first and second affinity reagents. Following their expression and purification, the targeting agents can be chemically conjugated to the protein vehicle. Many conjugation chemistries may be used to effect this conjugation, including homofunctional or heterofunctional linkers that yield ester, amide, thioether, carbon-carbon, or disulfide linkages.

In some embodiments, a peptide aptamer is included. A peptide aptamer is a peptide molecule that specifically binds to a target protein and interferes with the functional ability of that target protein. Peptide aptamers consist of a variable peptide loop attached at both ends of a protein scaffold. Such peptide aptamers can often have a binding affinity comparable to that of an antibody (nanomolar range). Due to the highly selective nature of peptide aptamers, they can be used not only to target a specific protein, but also to target specific functions of a given protein (e.g., a signaling function). Peptide aptamers are usually prepared by selecting the aptamer for its binding affinity with the specific target from a random pool or library of peptides. Peptide aptamers can be isolated from random peptide libraries by yeast two-hybrid screens. They can also be isolated from phage libraries or chemically generated peptides/libraries.

In certain embodiments, the payload is an adjuvant. In certain embodiments, the invention relates to any of the second compositions described herein, wherein the payload is an immunogen and an adjuvant: recruiting of professional antigen-presenting cells (APCs) to the site of antigen exposure; increasing the delivery of antigens by delayed/slow release (depot generation); immunomodulation by cytokine production (selection of Th1 or Th2 response); inducing T-cell response (prolonged exposure of peptide-MHC complexes [signal 1] and stimulation of expression of T-cell-activating co-stimulators [signal 2] on the APCs' surface) and targeting (e.g. carbohydrate adjuvants which target lectin receptors on APCs). Examples of adjuvants include, but are not limited to Freund's Complete Adjuvant, lipopolysaccharides, muramyldipeptide from TB, synthetic polynucleotides, aluminum hydroxide, aluminum phosphate, cytokines, and squalene.

### Antibodies, Antigen-Binding Domains, and Other Immunogens

As used herein, an “antibody,” an “antigen-binding site” or an or “affinity reagent,” is a molecule that binds to an antigen or receptor or another molecule. In some embodiments, an antibody, an antigen-binding site, an affinity reagent, or other immunogen is a molecule that specifically binds to an antigen or receptor or other molecule. In certain embodiments, some or all of an antibody, antigen-binding site, affinity reagent, or immunogen is composed of amino acids (including natural, non-natural, and modified amino acids), nucleic acids, or saccharides. In certain embodiments, an antigen-binding site, affinity reagent, or immunogen is a small molecule. In certain embodiments, antibodies specifically bind to molecules or targets, such as a cell surface antigen, a cell surface receptor, or other cell surface molecule.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, C-gamma-1 (Cγ1), C-gamma-2 (Cγ2), and C-gamma-3 (Cγ3). In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to VH, Cγ1, Cγ2, Cγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are known to one skilled in the art.

As used herein, the term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. As used herein, the term “antibody” refers to a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes or classes, respectively.

The term “antibody” is meant to include full-length antibodies and may refer to a natural antibody from any organism, an engineered antibody, or an antibody generated recombinantly for experimental, therapeutic, or other purposes as further defined below. Furthermore, full-length antibodies comprise conjugates as described and exemplified herein. As used herein, the term “antibody” comprises monoclonal and polyclonal antibodies. Antibodies can be antagonists, agonists, neutralizing, inhibitory, or stimulatory. Specifically included within the definition of “antibody” are full-length antibodies described and exemplified herein. By “full length antibody” herein is meant the structure that constitutes the natural biological form of an antibody, including variable and constant regions.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e. bi-specific) hybrid antibodies (e.g., Lanzavecchia et al., (1987) Eur. J. Immunol. 17:105) and in single chains (e.g., Huston et al. (1988) Proc. Natl. Acad. Sci. U.S.A. 85: 5879-5883 and Bird et al. (1988) Science 242: 423-426 (and related Erratum (1989) Science 244: 409), which are incorporated herein by reference). (See, generally, Hood et al., “Immunology”, Benjamin, N.Y., 2nd ed. (1984), and Hunkapiller et al. 1(1986) Nature 323: 15-16). Bispecific antibodies are a technique for creating a single polypeptide that binds to two different determinants. Bispecific antibodies may be made in many different formats, including but not limited to quadroma, F(ab′)2, tetravalent, heterodimeric scFv, bispecific scFv, tandem scFv, diabody and minibody formats, or scFvs appended to or recombinantly fused with whole antibodies.

The term “epitope” as used herein refers to a region of the antigen that binds to the antibody or antigen-binding fragment. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “Fc domain” encompasses the constant region of an immunoglobulin molecule. The Fc region of an antibody interacts with a number of Fc receptors and ligands, imparting an array of important functional capabilities referred to as effector functions, as described herein. For IgG the Fc region comprises Ig domains CH2 and CH3. An important family of Fc receptors for the IgG isotype are the Fc gamma receptors (FcγRs). These receptors mediate communication between antibodies and the cellular arm of the immune system.

As used herein, the term “Fab domain” encompasses the region of an antibody that binds to antigens. The Fab region is composed of one constant and one variable domain of each of the heavy and the light chains.

In one embodiment, the term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)2, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

(6) scFv-Fc, is produced in one embodiment, by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tri(a)body, a di- or tri-tandem scFv, a scFv-Fc bivalent molecule, an Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

As used herein, the terms “bivalent molecule” or “BV” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983); Morley (1980) Trends Pharm Sci. 463-468; Hudson et al. (1979) Int. J. Pept. Prot. Res. 14: 177-185; Spatola et al. (1986) Life Sci. 38: 1243-1249; Hann (1982) J. Chem. Soc. Perkin Trans. I 307-314; Almquist et al. (1980) J. Med. Chem. 23: 1392-1398; Jennings-White et al. (1982) Tetrahedron Lett. 23: 2533-2534; Szelke et al., European Application EP 45665 B1; Szelke et al. U.S. Pat. No. 4,424,407; Chemical Abstracts 97, 39405 (1982); Holladay et al. (1983) Tetrahedron Lett. 24: 4401-4404; and Hruby (1982) Life Sci. 31: 189-199).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (KD) is less than about 1×10-5 M or less than about 1×10-6 M or 1×10-7 M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In addition to antibody sequences, an antibody according to the present invention may comprise other amino acids, e.g., forming a peptide or polypeptide, such as a folded domain, or to impart to the molecule another functional characteristic in addition to ability to bind antigen. For example, antibodies of the invention may carry a detectable label, such as fluorescent or radioactive label, or may be conjugated to a toxin (such as a holotoxin or a hemitoxin) or an enzyme, such as beta-galactosidase or alkaline phosphatase (e.g., via a peptidyl bond or linker).

In one embodiment, an antibody of the invention comprises a stabilized hinge region. The term “stabilized hinge region” will be understood to mean a hinge region that has been modified to reduce Fab arm exchange or the propensity to undergo Fab arm exchange or formation of a half-antibody or a propensity to form a half-antibody. “Fab arm exchange” refers to a type of protein modification for human immunoglobulin, in which a human immunoglobulin heavy chain and attached light chain (half-molecule) is swapped for a heavy-light chain pair from another human immunoglobulin molecule. Thus, human immunoglobulin molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half-antibody” forms when a human immunoglobulin antibody dissociates to form two molecules, each containing a single heavy chain and a single light chain. In one embodiment, the stabilized hinge region of human immunoglobulin comprises a substitution in the hinge region.

In one embodiment, the term “hinge region” as used herein refers to a proline-rich portion of an immunoglobulin heavy chain between the Fc and Fab regions that confers mobility on the two Fab arms of the antibody molecule. It is located between the first and second constant domains of the heavy chain. The hinge region includes cysteine residues which are involved in inter-heavy chain disulfide bonds. In one embodiment, the hinge region includes cysteine residues which are involved in inter-heavy chain disulfide bonds.

In some embodiments, the present invention comprises a first component protein comprising a first binding pair partner and a second component protein comprising a second binding pair partner, wherein the binding pair partners comprise two protein moieties that form a heterodimer.

A “dimer” is a macromolecular complex formed by two macromolecules, usually proteins (or portions thereof) or nucleic acids (or portions thereof). A “homodimer” is formed by two identical macromolecules (“homodimerization”), while a “heterodimer” is formed by two distinct macromolecules (“heterodimerization”). Many dimers are non-covalently linked, but some (e.g., NEMO homodimers) can link via, e.g., disulfide bonds. Some proteins comprise regions specialized for dimerization, known as “dimerization domains.” In some instances, a truncated protein containing or comprising a dimerization domain (or two truncated proteins containing or comprising corresponding dimerization domains) may be able to interact in the absence of one or both complete protein sequence(s). Similarly, a fusion protein comprising a dimerization domain (or two fusion proteins comprising corresponding dimerization domains) may be able to interact in the absence of one or both complete protein sequence(s). Mutations to these domains may increase, or alternatively reduce, the formation of a dimer. Examples of macromolecules that can form dimers include, but are not limited to, proteins, nucleic acids, antibodies, receptor tyrosine kinases, proteins with leucine zippers, peptide Velcro, nuclear receptors, 14-3-3 proteins, G proteins, G protein-coupled receptors, transcription factors, kinesin, triosephosphate isomerase (TIM), alcohol dehydrogenase, Toll-like receptors, fibrinogen, tubulin, some glycoproteins, and some clotting factors. Additional examples of particular pairs include, but are not limited to, c-Jun/c-Fos, RelA (or c-Rel or RelB)/p50 (or p51) (Rel/NF-kappaB), AP-1, C/EBP, ATF/CREB, c-Myc, and NF-1.

The cell surface antigen may be any cell surface molecule that undergoes internalization, such as a protein, sugar, lipid head group or other antigen on the cell surface. Examples of cell surface antigens useful in the context of the invention include but are not limited to the transferrin receptor type 1 and 2, the EGF receptor (e.g., IMC-225), HER2/Neu (e.g., tastuzumab or pertuzumab), VEGF receptors, integrins, CD33, CD19, CD20, CD22, CD4 and the asialoglycoprotein receptor.

In certain embodiments, the construct relates to any of the compositions described herein, wherein the antibody is an anti-PD-1 antibody or wherein the antigen-binding site is an anti-PD-1 antigen-binding site. In certain embodiments, the construct relates to any of the compositions described herein, wherein the antibody is an anti-PD-1 antibody or wherein the antigen-binding site is an anti-PD-1 antigen-binding site, each either alone or in combination with either (a) an anti-PD-L1 antibody or an anti-PD-L1 antigen-binding site or (b) an anti-CTLA-4 antibody or an anti-CTLA-4 antigen binding site.

Lymph nodes (LN) are a critical cite of pathogenesis in immune-mediated diseases and cancer and are critical sites of targeting delivery of immunoregulatory molecules, check point inhibitors, and chemotherapy drugs. LN targeted delivery can markedly augment the therapeutic index of therapeutics, increasing their efficacy while reducing their toxicity. In certain embodiments, the construct relates to any of the composition described herein, wherein the targeting are Protin-101, antibody to anti-peripheral lymph node addressin (PNAd) or other lymph nodes targets.

Antibodies for use in the invention may be raised through any conventional method, such as through injection of immunogen into mice and subsequent fusions of lymphocytes to create hybridomas. Such hybridomas may then be used either (a) to produce antibody directly, which is purified and used for chemical conjugation to create a bispecific antibody, or (b) to clone cDNAs encoding antibody fragments for subsequent genetic manipulation. To illustrate one method employing the latter strategy, mRNA is isolated from the hybridoma cells, reverse-transcribed into cDNA using antisense oligo-dT or immunoglobulin gene-specific primers and cloned into a plasmid vector. Clones are sequenced and characterized. They may then be engineered according to standard protocols to combine the heavy and light chains of each antibody, separated by a short peptide linker, into a bacterial or mammalian expression vector as previously described to produce a recombinant bispecific antibody, which are then expressed and purified according to well-established protocols in bacteria or mammalian cells. Antibodies, or other proteinaceous affinity molecules or targeting agents such as peptides, may also be created through display technologies that allow selection of interacting affinity reagents through the screening of very large libraries of, for example, immunoglobulin domains or peptides expressed by bacteriophage. Antibodies may also be humanized through grafting of human immunoglobulin domains or made from transgenic mice or bacteriophage libraries that have human immunoglobulin genes/cDNAs.

In some embodiments, an antigen-binding domain may be comprised of proteinaceous structures other than antibodies that are able to bind to protein targets specifically, including but not limited to avimers, ankyrin repeats and adnectins, and other such proteins with domains that can be evolved to generate specific affinity for antigens, collectively referred to as “antibody-like molecules.” Modifications of proteinaceous affinity reagents through the incorporation of unnatural amino acids during synthesis may be used to improve their properties. Such modifications may have several benefits, including the addition of chemical groups that facilitate subsequent conjugation reactions. In some embodiments, the antigen-binding domain may be a peptide. In some embodiments, the peptide chain is a bispecific peptide. Peptides can readily be made and screened to create affinity reagents that recognize and bind to macromolecules such as proteins.

Bispecific affinity reagents may be constructed by separate synthesis and expression of the first and second affinity reagents. A polypeptide bispecific reagent can be expressed as two separately encoded chains that are linked by disulfide bonds during production in the same host cell, such as, for example, a bispecific scFv or diabody. Similarly, standard and widely used solid-phase peptide synthesis technology can be used to synthesize peptides, and chimeric bispecific peptides are well known in the art. A bispecific peptide strategy may be used to combine the first and second first and second affinity reagents in a single peptide chain. Alternatively, polypeptide chains or peptide chains can be expressed/synthesized separately, purified and then conjugated chemically to produce the bispecific affinity reagents useful in the compositions and methods described herein. Many different formats of antibodies may be used. Whole antibodies, F(ab′)2, F(ab′), scFv, as well as smaller Fab and single-domain antibody fragments may all be used to create the first and second affinity reagents. Following their expression and purification, the targeting agents can be chemically conjugated to the protein vehicle. Many conjugation chemistries may be used to effect this conjugation, including homofunctional or heterofunctional linkers that yield ester, amide, thioether, carbon-carbon, or disulfide linkages.

In some embodiments, a peptide aptamer is included. A peptide aptamer is a peptide molecule that specifically binds to a target protein and interferes with the functional ability of that target protein. Peptide aptamers consist of a variable peptide loop attached at both ends of a protein scaffold. Such peptide aptamers can often have a binding affinity comparable to that of an antibody (nanomolar range). Due to the highly selective nature of peptide aptamers, they can be used not only to target a specific protein, but also to target specific functions of a given protein (e.g., a signaling function). Peptide aptamers are usually prepared by selecting the aptamer for its binding affinity with the specific target from a random pool or library of peptides. Peptide aptamers can be isolated from random peptide libraries by yeast two-hybrid screens. They can also be isolated from phage libraries or chemically generated peptides/libraries.

In some embodiments, a nucleic acid aptamer is included. Nucleic acid aptamers are nucleic acid oligomers that bind other macromolecules specifically; such aptamers that bind specifically to other macromolecules can be readily isolated from libraries of such oligomers by technologies such as SELEX.

In some embodiments, an oligosaccharide is included. Certain oligosaccharides are known ligands for certain extracellular or cell surface receptors.

In one embodiment, the antibody or antigen-binding fragment binds its target with a KD of 0.1 nM-10 mM. In one embodiment, the antibody or antigen-binding fragment binds its target with a KD of 0.1 nM-1 mM. In one embodiment, the antibody or antigen-binding fragment binds its target with a KD within the 0.1 nM range. In one embodiment, the antibody or antigen-binding fragment binds its target with a KD of 0.1-2 nM. In another embodiment, the antibody or antigen-binding fragment binds its target with a KD of 0.1-1 nM. In another embodiment, the antibody or antigen-binding fragment binds its target with a KD of 0.05-1 nM. In another embodiment, the antibody or antigen-binding fragment binds its target with a KD of 0.1-0.5 nM. In another embodiment, the antibody or antigen-binding fragment binds its target with a KD of 0.1-0.2 nM.

In some embodiments, the antibody or antigen-binding fragment thereof provided herein comprises a modification. In another embodiment, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is one as further defined herein below. In some embodiments, the modification is a N-terminus modification. In some embodiments, the modification is a C-terminal modification. In some embodiments, the modification is an N-terminus biotinylation. In some embodiments, the modification is a C-terminus biotinylation. In some embodiments, the secretable form of the antibody or antigen-binding fragment comprises an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises a C-terminal modification that allows binding to an enzymatically biotinylatable site. In some embodiments, biotinylation of said site functionalizes the site to bind to any surface coated with streptavidin, avidin, avidin-derived moieties, or a secondary reagent.

It will be appreciated that the term “modification” can encompass an amino acid modification such as an amino acid substitution, insertion, and/or deletion in a polypeptide sequence.

In one embodiment, a variety of radioactive isotopes are available for the production of radioconjugate antibodies and other proteins and can be of use in the methods and compositions provided herein. Examples include, but are not limited to, At21, Cu64, I131, I125, Y90, Re186, Re188, Sm153, Bi212, P32, Zr89, F-18, I-124 and radioactive isotopes of Lu. In a further embodiment, the amino acid sequences of the invention may be homologues, variants, isoforms, or fragments of the sequences presented. The term “homolog” as used herein refers to a polypeptide having a sequence homology of a certain amount, namely of at least 70%, e.g. at least 80%, 90%, 95%, 96%, 97%, 98%, 99% of the amino acid sequence it is referred to. Homology refers to the magnitude of identity between two sequences. Homolog sequences have the same or similar characteristics, in particular, have the same or similar property of the sequence as identified. The term ‘variant’ as used herein refers to a polypeptide wherein the amino acid sequence exhibits substantially 70, 80, 95, or 99% homology with the amino acid sequence as set forth in the sequence listing. It should be appreciated that the variant may result from a modification of the native amino acid sequences, or by modifications including insertion, substitution or deletion of one or more amino acids. The term “isoform” as used herein refers to variants of a polypeptide that are encoded by the same gene, but that differ in their isoelectric point (pI) or molecular weight (MW), or both. Such isoforms can differ in their amino acid composition (e.g. as a result of alternative splicing or limited proteolysis) and in addition, or in the alternative, may arise from differential post-translational modification (e.g., glycosylation, acylation, phosphorylation deamidation, or sulphation). As used herein, the term “isoform” also refers to a protein that exists in only a single form, i.e., it is not expressed as several variants. The term “fragment” as used herein refers to any portion of the full-length amino acid sequence of protein of a polypeptide of the invention which has less amino acids than the full-length amino acid sequence of a polypeptide of the invention. The fragment may or may not possess a functional activity of such polypeptides.

In an alternate embodiment, enzymatically active toxin or fragments thereof that can be used in the compositions and methods provided herein include, but are not limited, to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

A chemotherapeutic or other cytotoxic agent may be conjugated to the protein, according to the methods provided herein, as an active drug or as a prodrug. The term “prodrug” refers to a precursor or derivative form of a pharmaceutically active substance that is less cytotoxic to tumor cells compared to the parent drug and is capable of being enzymatically activated or converted into the more active parent form. (See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985.) The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as provided herein include but are not limited to any of the aforementioned chemotherapeutic.

### Linkers and Tethers

In some embodiments, the chimeric protein construct comprises a fusion protein, e.g., encoded by a transgenic nucleic acid sequence expressing the non-clathrin protein moiety comprising a CAR. In some embodiments, the chimeric protein construct comprises a fusion protein, e.g., encoded by a transgenic nucleic acid sequence expressing a clathrin moiety and a CAR. In some embodiments, the chimeric protein construct further comprises a linker or tether operably linking the clathrin protein moiety and the CAR. The clathrin moiety is attached, e.g., directly or via a tether or linker, to the CAR construct of interest.

Linkers may attach a clathrin moiety to a non-clathrin moiety and/or may attach two non-clathrin moieties. In some embodiments, a clathrin moiety is attached to a CAR-T or CAR-Treg via a linker. In some embodiments, a clathrin moiety is attached to an antibody via a linker while also being attached to a CAR-T or CAR-Treg via a linker. Various combinations of embodiments are possible. In some embodiments, the compositions are 1. CL-PL 2, CL-CAR, 3, CL-CAR-PL, 4. CAR-CL-AB, in each case attached by linkers or tethers, or fused in any order (CL=clathrin; PL=payload; AB=antibody; CAR=chimeric antibody).

In certain embodiments, linkers (also known as “tethers,” “linker molecules,” “cross-linkers,” or “spacers”) may be used to conjugate, e.g., the clathrin protein moiety or derivative to the CAR, the payload to an element of the chimeric protein construct, and/or the antibody to an element of the chimeric protein construct.

The majority of known cross-linkers react with amine, carboxyl, and sulfhydryl groups. Tethers/linker molecules may be responsible for different properties of the composition. The length of the tether or linker should be considered in light of molecular flexibility during the conjugation step, and the availability of the conjugated molecule for its target. Longer tethers or linkers may thus improve the biological activity of the compositions of the invention, as well as the ease of preparation of them. The geometry of the tether or linker may be used to orient a molecule for optimal reaction with a target. A tether or linker with flexible geometry may allow the entire composition to conformationally adapt as it binds a target sequence. The nature of the tether or linker may be altered for other various purposes. For example, the hydrophobicity of a polymeric linker may be controlled by the order of monomeric units along the polymer, e.g. a block polymer in which there is a block of hydrophobic monomers interspersed with a block of hydrophilic monomers.

There are many options for linking modules, including linking the clathrin to the CAR or other non-clathrin moiety. A variety of linkers may find use in the compositions and methods provided herein to generate conjugates. The term “linker”, “linker sequence”, “spacer”, “tethering sequence” or grammatical equivalents thereof refer to a molecule or group of molecules (such as a monomer or polymer) that connects two molecules and often serves to place the two molecules in a preferred configuration. A number of strategies may be used to covalently link molecules together. These include, but are not limited to, polypeptide linkages between N- and C-terminus of proteins or protein domains, linkage via disulfide bonds, and linkage via chemical cross-linking reagents. In one aspect of this embodiment, the linker is a peptide bond, generated by recombinant techniques or peptide synthesis. In another embodiment the linker is a cysteine linker. In yet another embodiment it is a multi-cysteine linker. Choosing a suitable linker for a specific case where two polypeptide chains are to be connected depends on various parameters, including but not limited to the nature of the two polypeptide chains (e.g., whether they naturally oligomerize), the distance between the N- and the C-termini to be connected if known, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 30 amino acid residues. In one embodiment, the linker is from about 1 to 30 amino acids in length. In another embodiment, the linker is from about 1 to 15 amino acids in length. In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. Useful linkers include glycine-serine polymers, glycine-alanine polymers, alanine-serine polymers, and other flexible linkers such as the tether for the shaker potassium channel, and a large variety of other flexible linkers, as will be appreciated by those in the art. Suitable linkers may also be identified by screening databases of known three-dimensional structures for naturally occurring motifs that can bridge the gap between two polypeptide chains. In one embodiment, the linker is not immunogenic when administered in a human subject. Thus, linkers may be chosen such that they have low immunogenicity or are thought to have low immunogenicity. Another way of obtaining a suitable linker is by optimizing a simple linker, e.g., (Gly4Ser)n, through random mutagenesis. Alternatively, once a suitable polypeptide linker is defined, additional linker polypeptides can be created to select amino acids that more optimally interact with the domains being linked. Other types of linkers that may be used in the compositions and methods provided herein include artificial polypeptide linkers and inteins. In another embodiment, disulfide bonds are designed to link the two molecules. In another embodiment, linkers are chemical cross-linking agents. For example, a variety of bifunctional protein coupling agents may be used, including but not limited to N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). In another embodiment, chemical linkers may enable chelation of an isotope. For example, Carbon-14-labeled 1-isothiocyanatobenzyl-3-methyldiethylene triaminepentaacetic acid (MX-DTPA) is an exemplary chelating agent for conjugation of radionucleotide to the antibody. The linker may be cleavable, facilitating release of the cytotoxic drug in the cell. For example, an acid-labile linker, peptidase-sensitive linker, dimethyl linker or disulfide-containing linker (Chari et al. (1992) Cancer Research 52: 127-131 [“Chari 1992” ]) may be used. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use to link the components of the conjugates of the compositions and methods provided herein.

The chemistry of preparing and utilizing a wide variety of molecular linkers or tethers is well-known in the art and many pre-made linkers for use in conjugating molecules are commercially available from vendors such as Pierce Chemical Co., Roche Molecular Biochemicals, United States Biological. Exemplary linker molecules or tethers for use in the compositions of the invention include, but are not limited to: aminocaproic acid (ACA); polyglycine, and any other amino acid polymer, polymers such as polyethylene glycol (PEG), polymethyl methacrylate (PMMA), polypropylene glycol (PPG); homobifunctional reagents such as APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS; heterobifunctional reagents such as ABH, AEDP, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, MBuS, M2C2H, MPBH, MSA, NHS-ASA, PDPH, PMPI, SADP, SAED SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS. Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS; and trifunctional linkers such as Sulfo-SBED.

Branched linkers or tethers may be prepared or used so that multiple moieties per linker are able to react. Such multiply reactive linkers or tethers allow the creation of multimeric binding sites.

An appropriate tether or linker may be a macromolecular polymer. Any of the above-mentioned polymers may comprise the macromolecular polymer. In certain embodiments, such macromolecular polymers may be comprised entirely of one type of polymeric molecule. In other embodiments, the macromolecular polymers may be comprised of more than one type of polymeric molecule. The macromolecular polymers may exist in many possible structures, for example, linear, comb-branched, dendrigraft, dendrimer, or a linear dendron architectural copolymer. For example, polyethylene glycol (PEG) and polypropylene glycol (PPG) may be used to create a variety of bi- and multivalent linkers. Methods of synthesizing, activating, and modifying branched PEG/PPG polymers and PEG/PPG block co-polymers are well-known in the art. PEG is hydrophilic, while PPG is hydrophobic. For instance, a tether or linker could be synthesized with a PPG core and PEG branches.

### Exemplary Methods of Production and Use

Provided herein are methods of producing a CAR-expressing target cell of interest in vivo. In some embodiments, the methods comprise providing a chimeric protein construct comprising: (a) a clathrin protein moiety or functional derivative thereof and a CAR comprising an ectodomain comprising an antigen-binding domain; a transmembrane domain; and an endodomain comprising an intracellular signaling domain; (b) administering the chimeric protein construct to the subject; and (c) transducing a target cell of interest in the subject with the chimeric protein construct to produce a CAR-expressing cell of interest. In some embodiments, transducing the target cell of interest comprises clathrin-mediated endocytosis of the CAR. In some embodiments, the clathrin and the CAR are linked.

In some embodiments, an antigen-binding domain of the CAR selectively binds to a T-cell or other immune effector cell. The CAR is internalized via clathrin-mediated endocytosis, and the CAR is transported to the surface of the T-cell or other immune effector cell in response to an appropriate intracellular signaling domain on the CAR. A second antigen-binding domain of the CAR selectively binds to a second target cell of interest, bringing the T-cell or other immune effector cell into close proximity with the second cell of interest.

In other embodiments, the chimeric protein construct comprises a non-CAR antigen-binding protein (e.g., linked to the clathrin, the CAR, or as a separately linked protein or antibody). The non-CAR antigen-binding protein selectively binds to a T-cell or other immune effector cell. The CAR is internalized via clathrin-mediated endocytosis, and the CAR is transported to the surface of the T-cell or other immune effector cell in response to an appropriate intracellular signaling domain on the CAR. An antigen-binding domain of the CAR selectively binds to a target cell of interest.

In some embodiments, an antigen-binding domain of the CAR selectively binds to a target cell of interest (e.g., to a tumor or other neoplastic cell or to a cancer cell). The CAR is internalized via clathrin-mediated endocytosis, and the CAR is transported to the surface of the cell in response to an appropriate intracellular signaling domain on the CAR. A second antigen-binding domain of the CAR selectively binds to a second target cell of interest (e.g., an immune effector cell or a regulatory cell) or to a pharmaceutical composition (e.g., a cytotoxic drug, a biomarker, or an imaging agent).

In some embodiment, the chimeric protein construct comprises a non-CAR antigen-binding protein (e.g., linked to the clathrin, the CAR, or as a separately linked protein or antibody). The non-CAR antigen-binding protein selectively binds to a target cell of interest (e.g., to a tumor or other neoplastic cell or to a cancer cell). The CAR is internalized via clathrin-mediated endocytosis, and the CAR is transported to the surface of the cell in response to an appropriate intracellular signaling domain on the CAR. An antigen-binding domain of the CAR selectively binds to a second target cell of interest (e.g., an immune effector cell or a regulatory cell) or to a pharmaceutical composition (e.g., a cytotoxic drug, a biomarker, or an imaging agent).

Provided herein are in vivo methods of producing immune effector cells having CAR surface proteins. In some embodiments, a transduced immune effector cell (e.g., a CAR-T-cell) can mount an immunological response against a mutant cell; a diseased cell; a tumor or other neoplastic cell; a cancer cell; a cell that promotes growth, mutagenesis, or metastasis of a tumor cell or other neoplastic cell; a cell expressing a specific marker of interest; a regulatory cell; a secretory cell; a cell that inhibits or promotes cell death; another immune effector cell; a cell that regulates an immune effector cell; or a cell belonging to an infectious agent.

Also provided herein are in vivo methods of producing cells having CAR surface proteins (e.g., comprising an antigen-binding receptor specific for a selected external stimulus or for an antigen significant for the selected external stimulus), the cells having CAR surface proteins thereby having an improved response to a selected external stimulus. In some embodiments, a transduced cell is, e.g., a mutant cell; a diseased cell; a tumor or other neoplastic cell; a cancer cell; a cell that promotes growth, mutagenesis, or metastasis of a tumor cell or other neoplastic cell; a cell expressing a specific marker of interest; a regulatory cell; a secretory cell; a cell that inhibits or promotes cell death; an immune effector cell; a cell that regulates an immune effector cell; or a cell belonging to an infectious agent. The cell having CAR surface proteins is then exposed to the selected external stimulus (e.g., an imaging agent, a biomarker, or a pharmaceutical composition).

Provided herein are methods of producing a chimeric antigen receptor (CAR)-expressing immune effector cell in vivo by administering the chimeric protein construct to a subject and transducing an immune effector cell in the subject with the chimeric protein construct to produce a CAR-expressing immune effector cell. In some embodiments, the immune effector cell comprises a T-cell (e.g., a regulatory T-cell [Treg]), a B-cell, a dendritic cell, or a natural killer cell (NK).

Also provided herein are methods of inhibiting the growth, mutagenesis or metastasis of a tumor or other neoplasm in a subject in need thereof, comprising obtaining the antigenic profile of the tumor or other neoplasm, providing an antigen binding domain that binds an antigen specific to the tumor or other neoplasm or binds an antigen specific to a target cell of interest (e.g., that promotes growth, mutagenesis, or metastasis of the tumor or other neoplasm), providing a chimeric protein construct with the antigen binding domain, administering the chimeric protein construct to the subject, and inhibiting the growth, mutagenesis or metastasis of the tumor or other neoplasm. In some embodiments, the method further comprises transducing an immune effector cell in the subject with the chimeric protein to yield a CAR-expressing immune effector cell in the subject and expanding the CAR-expressing immune effector cell in the subject to obtain a population of CAR-expressing immune effector cells.

In some embodiments, the biological activity is altered compared with a corresponding control protein.

In some embodiments, delivery is targeted to a lymph node or lymph node venule.

A skilled artisan would recognize that the term “biological activity” refers to any activity associated with a protein that can be measured by an assay. In some embodiments, an altered biological activity comprises increased enzyme activity. In some embodiments, an altered biological activity comprises decreased enzyme activity. In some embodiments, an altered biological activity comprises increased stability of the polypeptide. In some embodiments, an altered biological activity comprises decreased stability of the polypeptide.

In some embodiments, the subject or patient is a bird or a mammal. In some embodiments, the subject or patient is a human.

In some embodiments, the CAR-expressing cell of interest treats or alleviates a disease or an abnormal physiological condition. Examples of diseases and/or abnormal physiological conditions include, but are not limited to, a tumor, a cancer/other neoplasm, and neuroinflammation or a neurodegenerative disease (such as but not limited to Alzheimer's Disease, Parkinson's Disease, amyotrophic lateral sclerosis [ALS], Progressive Supranuclear Palsy [PSP], Frontotemporal Dementia [FTD], Corticobasal degeneration [CBD], multiple sclerosis [MS], or a prion disease).

A “neoplasm” is a type of abnormal and excessive growth (“neoplasia”) of tissue or cells. The growth of a neoplasm is uncoordinated with that of the normal surrounding tissue, and it persists growing abnormally, even if the original trigger is removed. This abnormal growth usually (but not always) forms a mass, typically known as a “tumor.” A neoplasm or a tumor may be benign (e.g., a cyst), potentially malignant (“precancerous”), or malignant (“cancerous”).

A cancerous neoplasm may be referred to as a “cancer.” “Cancer” may also refer to the medical disease/condition of having a cancerous neoplasm. “Cancer” is a disease caused by uncontrolled cell division. Cancer may “metastasize” or spread from the location of the initial (“primary”) malignancy to a “secondary” location. Types of cancers include, but are not limited to, sarcoma, bladder, brain, breast, colon, rectal, endometrial, kidney, leukemia (e.g., acute lymphoblastic leukemia, chronic lymphocytic leukemia), multiple myeloma, liver, lung, lymphoma (e.g., Hodgkin's or non-Hodgkin's), retinoblastoma, skin (e.g., melanoma, basal cell, squamous cell), ovarian, pancreatic (e.g., pancreatic ductal adenocarcinoma), prostate, thyroid, and uterine cancers.

A “sarcoma” is a cancer that arises from transformed cells of mesenchymal (connective tissue) origin, including, but not limited to, bone, cartilage, fat, muscle, vascular, or hematopoietic tissues.

“Hematologic” cancers include leukemias, lymphomas, and multiple myeloma.

“Neurodegeneration” involves progressive loss of structure or function of neurons and/or neuronal death. Neurodegenerative processes may result in neurodegenerative diseases. Other types of degeneration include, but are not limited to, age-related neurodegeneration, injury-related neurodegeneration (e.g., from physical, chemical, or other types of injury), neurodegeneration resulting from an infectious agent (e.g., prion, virus, bacterium, or parasite), and/or neurodegeneration caused by genetic mutation. Some neurodegenerative diseases are “proteopathies” associated with aggregation of misfolded proteins (e.g., prion, alpha-synuclein, tau, beta amyloid). Neurodegenerative diseases may involve aberrant protein degradation pathways, mitochondrial dysfunction, DNA damage, aberrant cell death. Neurodegeneration ranges from molecular to systemic. Cognitive performance may decrease in working, spatial, and episodic memory and/or in processing speed.

In some embodiments, the neurodegenerative disease is any one of the group of neurodegenerative diseases consisting of an Alzheimer's disease (AD) or AD dementia, a frontotemporal dementia (FTD), a vascular dementia, a Lewy body dementia (LBD), a dementia with Lewy bodies (DLB), Huntington's disease, Batten disease, a Parkinson's disease (PD) or PD dementia (PDD), an amyotrophic lateral sclerosis (ALS) or ALS dementia, or a prion disease or prion disease dementia. In some embodiments, wherein the neurodegenerative disease is Alzheimer's disease (AD) or AD dementia. In other embodiments, the neurodegenerative disease is Parkinson's disease (PD) or PD dementia (PDD). In still other embodiments, the neurodegenerative disease is amyotrophic lateral sclerosis (ALS) or ALS dementia. In yet other embodiments, the neurodegenerative disease is a prion disease or prion disease dementia.

In some embodiments, the CAR targets a cytokine having a neurodegenerative or neuroinflammatory activity.

In some embodiments, a clathrin/CAR construct is administered to a subject, such as by injection or by oral, subcutaneous, intravenous, intranasal, intraotical, transdermal, topical (e.g., gels, salves, lotions, creams, etc.), intraperitoneal, intramuscular, intrapulmonary, vaginal, parenteral, rectal, or intraocular administration. The clathrin/CAR construct activate a T cell, a tumor cell, a neuron, or another cell type of interest.

Alternatively, in some embodiments, one or more CAR moieties and one or more clathrin moieties are attached, either individually or as clathrin/CAR constructs, and the combination is administered to a subject, such as by injection or by oral, subcutaneous, intravenous, intranasal, intraotical, transdermal, topical (e.g., gels, salves, lotions, creams, etc.), intraperitoneal, intramuscular, intrapulmonary, vaginal, parenteral, rectal, or intraocular administration. The combination activate a T cell, a tumor cell, a neuron, or another cell type of interest of the immune cells to achieve their protective (e.g., lowering neuroinflammation) or cell killing (e.g., tumor cells).

In some embodiments, the chemotherapeutic agent is a nucleoside analog. In some embodiments, the chemotherapeutic agent interferes with the normal function of cell division (e.g., by interference with the function of microtubules).

In some embodiments, the chemotherapeutic agent is selected from the group consisting of gemcitabine or a derivative thereof, paclitaxel or a derivative thereof, carboplatin or a derivative thereof, and cisplatin or a derivative thereof.

In some embodiments, the chemotherapeutic agent is gemcitabine (e.g., 2′,2′-difluoro 2′-deoxycitidine [dFdC]; GEMZAR™ [LILLY USA™]) or a derivative thereof, a nucleoside analog (cytidine analog) and metabolic inhibitor that blocks manufacture of new DNA to result in cell death of dividing cell. Compositions and/or constructs comprising gemcitabine are used, e.g., for the treatment of ovarian cancer, breast cancer, non-small cell lung cancer, pancreatic cancer, or cholangiocarcinoma or other biliary tract cancers. In some embodiments, constructs and/or compositions comprising gemcitabine are used alone. In some embodiments, constructs and/or compositions comprising gemcitabine are used, e.g., in combination with other pharmaceuticals, including, but not limited to, other chemotherapeutic agents (e.g., paclitaxel, carboplatin, cisplatin). Other combinations include, but are not limited to, a platinum-based therapy or a gold-based therapy.

In some embodiments, the chemotherapeutic agent is a taxane or a derivative thereof. Examples of taxanes and their derivatives include, but are not limited to, paclitaxel, docetaxel, protein-bound paclitaxel, 10-deacetylbaccatin III, baccatin III, paclitaxel C, or 7-epipaclitaxel. In some embodiments, the taxane is paclitaxel (TAXOL® [BRISTOL-MYERS SQUIBB™]; NSC 125973; (1S,2A, 3R, 4S, 7R, 9S, 10S, 12R, 15S)-4,12-diacetoxy-15-{[(2R,3S)-3-(benzoylamino)-2-hydroxy-3-phylpropanoyl]oxy}-1,9-dihydroxy-10, 14, 17, 17-tetramethyl-11-oxo-6-oxatetracyclo[11.3.1.0˜3, 10˜.0˜4,7˜]heptadec-13-en-2-yl rel-benzoate), protein-bound paclitaxel (nanoparticle albumin-bound paclitaxel, nabpaclitaxel; ABRAXANE™ [CELGENE™]), or docetaxel (TAXOTERE™ [SANOFI-AVENTIS US™]; 3421782; DOCEFREZ™ [SUN PHARMA GLOBAL™]; ZYTAX™ [ZYDUS™]; [(1S,2S,3R,4S,7R,9S,10S,12R,15S)-4-acetyloxy-1,9,12-trihydroxy-15-[(2R,3S)-2-hydroxy-3-[(2-methylpropan-2-yl)oxycarbonylamino]-3-phenylpropanoyl]oxy-10, 14, 17, 17-tetramethyl-11-oxo-6-oxatetracyclo[11.3.1.03,10.04,7]heptadec-13-en-2-yl] benzoate). Compositions and/or constructs comprising paclitaxel or another taxane are used, e.g., for the treatment of ovarian cancer, cervical cancer, breast cancer, lung cancer (including non-small-cell lung cancer), bladder cancer, prostate cancer, pancreatic cancer, adenocarcinoma, melanoma, esophageal cancer, or other types of solid tumor cancers as well as Kaposi's sarcoma. Compositions and/or constructs comprising docetaxel or another taxane are used, e.g., for the treatment of breast, colorectal, lung, ovarian, prostate, liver, renal, gastric, or head and neck cancers or melanoma or adenocarcinoma. In some embodiments, constructs and/or compositions comprising a paclitaxel, docetaxel, or another taxane are used alone. In some embodiments, constructs and/or compositions comprising paclitaxel, docetaxel, or another taxane are used, e.g., in combination with other pharmaceuticals, including, but not limited to, other chemotherapeutic agents.

As noted in the U.S. Food & Drug Administration (FDA) label for TAXOL® (paclitaxel) Injection (Patient Information Included) (Ref. ID No. 2939751; BRISTOL-MYERS SQUIBB™) (“TAXOL® FDA Label”), which is incorporated in its entirety herein by reference, paclitaxel is a natural product obtained via semi-synthetic process from Taxus baccata (common yew; European yew) and having an anti-tumor activity. (Paclitaxel is also isolated or synthesized from Taxus brevifolia [Pacific yew] and other yew species, including, but not limited to, wild yew species in India and China.) Its chemical name is 5beta, 20-epoxy-1,2alpha, 4,7beta, 10beta, 13alpha-hexahydroxytax-11-en-9-one 4,10-diacetate 2-benzoate 13-ester with (2R,3S)—N-benzoyl-3-phenylisoserine (5β,20-epoxy-1,2α,4,7β,10β,13α-hexahydroxytax-11-en-9-one 4,10-diacetate 2-benzoate 13-ester with 2R,3S)—N-benzoyl-3-phenylisoserine; C47H51NO14; molecular weight 853.9; melting point 216-217° C.), and it has the chemical structure:

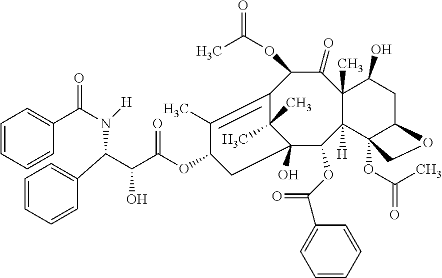

A lipophilic antimicrotubule agent, paclitaxel promotes assembly of microtubules and stabilizes microtubules by prevent depolymerization, resulting in inhibition of the normal reorganization of the microtubule network necessary for critical interphase and mitotic cellular functions, as well as inducing abnormal microtubule arrays throughout the cell cycle and multiple asters of microtubules during mitosis (TAXOL® FDA Label). In the wake of intravenous administration of TAXOL®, paclitaxel plasma concentrations have been observed to decline in a biphasic manner (TAXOL® FDA Label).

In some embodiments, a combination of the protein with the biological active agents specified above, i.e., a cytokine, an enzyme, a chemokine, a radioisotope, an enzymatically active toxin, or a chemotherapeutic agent can be applied.

In some embodiments, a variety of other therapeutic agents may find use for administration with the antibodies and conjugates of the compositions and methods provided herein. In some embodiments, the antibodies of the present invention are glycosylated. Antibodies contain carbohydrate structures at conserved positions in the heavy chain constant regions, with each isotype possessing a distinct array of N-linked carbohydrate structures, which variably affect protein assembly, secretion or functional activity. (Wright, A., and Morrison, S. L., Trends Biotech. 15:26-32 (1997)). In the context of antibodies, the oligosaccharides attached to each heavy chain may be the same or different.

In some embodiments, the antibodies of the present invention comprise one or more antibody isoforms. Several types of antibody isoforms are known in the art, including, inter alia, sequence isoforms and charge isoforms. One sequence antibody isoform is generated through heavy chain terminal lysine processing. Although human IgG heavy chain genes encode a C-terminal lysine, it is rapidly lost in vivo, and is mostly absent from the antibodies isolated from serum. Thus, in some embodiments, the antibodies of the invention lack the heavy chain C-terminal lysine residues.

In other embodiments, the antibodies of the present invention comprise multiple antibody charge isoforms. The antibody preparations can have several isoforms at comparable levels or a single primary charge isoform and one or more secondary charge isoform. Thus, in one embodiment, the antibodies of the present invention comprise a main charge isoform and secondary charge isoforms. In some embodiments, the term “secondary isoforms” refers to the set of charged isoforms that cumulatively amount to less than 30% of all isoforms present in an antibody preparation. In embodiments, the secondary charge isoforms comprise at most 30% of an antibody preparation. In other embodiments, the secondary charge isoforms comprise at most 25% of an antibody preparation. In other embodiments, the secondary charge isoforms comprise at most 20% of an antibody preparation. In other embodiments, the secondary charge isoforms comprise at most 15% of an antibody preparation. In other embodiments, the secondary charge isoforms comprise at most 10% of an antibody preparation. In other embodiments, the secondary charge isoforms comprise at most 5% of an antibody preparation. In some embodiments, the secondary charge isoforms are negatively charged (acidic) relative to the main isoform. In other embodiments, the secondary charge isoforms are positively charged (basic) relative to the main isoform. In further embodiments, the secondary charge isoforms comprise species that are both are positively charged (basic) and negatively charged (acidic) relative to the main isoform.

In some embodiments, the secondary isoforms have the same antigen affinity as the main isoform. In other embodiments, the secondary isoforms have higher antigen affinity as compared to the main isoform.

A “subject” for the purposes of the compositions and methods provided herein includes humans and other animals, preferably mammals and most preferably humans. In another embodiment the subject is a mammal, and in yet another embodiment the subject is human.

By “condition” or “disease” herein are meant a disorder that may be ameliorated by the administration of a pharmaceutical composition comprising the conjugate of the compositions and methods provided herein.

In some embodiments, the term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

In some embodiments, the protein moiety or moieties of the present invention, including the CAR, is constructed, e.g., by using a nucleic acid vector.

In one embodiment, provided herein are primers used for amplification and construction of the vectors and nucleic acids provided herein. It is to be understood by a skilled artisan that other primers can be used or designed to arrive at the vectors, nucleic acids and conjugates provided herein.

In one embodiment, provided herein is a vector comprising the nucleic acid encoding for the conjugate components provided herein. In another embodiment, the vector comprises nucleic acid encoding the protein, polypeptides, peptides, antibodies, and recombinant fusions provided herein. Modifications to the nucleic acid encoding the protein can be used to introduce a modification, truncation, or elongation of the expressed protein.

In some embodiments, the conjugates are purified or isolated after expression and prior to administration (e.g., CAR either before and/or after addition of the clathrin moiety; antibodies either before and/or after their addition). Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of conjugates. For example, the bacterial proteins A and G bind to the Fc region. Likewise, the bacterial protein L binds to the Fab region of some antibodies, as of course does the antibody's target antigen. Purification can often be enabled by a particular fusion partner. For example, proteins may be purified using glutathione resin if a GST fusion is employed, Ni+2 affinity chromatography if a His-tag is employed, or immobilized anti-flag antibody, if a flag-tag is used. The degree of purification necessary will vary depending on the screen or use of the conjugates. In some instances, no purification is necessary. For example, in one embodiment, if the conjugates are secreted, screening may take place directly from the media. As is well known in the art, some methods of selection do not involve purification of proteins. Thus, for example, if a library of conjugates is made into a phage display library, protein purification may not be performed.

Pharmaceutical compositions are contemplated wherein the compositions and methods provided herein and one or more therapeutically active agents are formulated. Formulations of the conjugates of the compositions and methods provided herein are prepared for storage by mixing said a conjugated protein having the desired degree of purity with optional pharmaceutically acceptable carriers, excipients or stabilizers, in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants or polyethylene glycol (PEG). In another embodiment, the pharmaceutical composition that comprises the conjugate of the compositions and methods provided herein is in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

The conjugate molecules disclosed herein may also be formulated as immunoliposomes. A liposome is a small vesicle comprising various types of lipids, phospholipids and/or surfactant that is useful for delivery of a therapeutic agent to a mammal. Liposomes containing the conjugates are prepared by methods known in the art, such as described in Eppstein et al. (1985) PNAS 82:3688-3692; Hwang et al. (1980) PNAS 77:4030-4034; U.S. Pat. Nos. 4,485,045; 4,544,545; and WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al. (1989) J. National Cancer Inst. 81:1484).

The conjugate molecules provided herein may also be entrapped in microcapsules prepared by methods including but not limited to coacervation techniques, interfacial polymerization (for example using hydroxymethylcellulose or gelatin-microcapsules, or poly-(methylmethacrylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers (which are injectable microspheres composed of lactic acid-glycolic acid copolymer and leuprolide acetate), and poly-D-(−)-3-hydroxybutyric acid) which is a microsphere-based delivery system composed of the desired bioactive molecule incorporated into a matrix of poly-DL-lactide-co-glycolide (PLG).

Administration of the pharmaceutical composition comprising the conjugates provided herein, preferably in the form of a sterile aqueous solution, may be done in a variety of ways, including, but not limited to orally, sublingually, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. As is known in the art, the pharmaceutical composition may be formulated accordingly depending upon the manner of introduction.

The term “subject” refers in one embodiment to a mammal including a human in need of therapy for, or susceptible to, a condition or its sequelae. The term “subject” refers in another embodiment to an avian or a bird in need of therapy for, or susceptible to, a condition or its sequelae. The term “subject” does not exclude an individual that is normal in all respects.

“Mammals” (class “Mammalia”) are endothermic vertebrates usually characterized by the presence of hair, three middle-ear bones, a neocortex, and in female mammals, mammary glands that secrete milk during lactation. With a few exceptions, mammals are viviparous. Mammals include, but are not limited to, humans, dogs, cats, rats, mice, bats, pigs, cows/cattle, buffalo, goats, sheep, camels, dromedaries, donkeys, horses, reindeer, yaks, moose, bison, bison/cow hybrids, lions, tigers, panda bears, leopards, giraffes, whales, and dolphins.

“Birds” or “avians” (class “Aves”), also known as avian dinosaurs, areendothermic vertebrates, usually characterized by feathers, toothless beaked jaws, the laying of hard-shelled eggs, a high metabolic rate, a four-chambered heart, and a strong yet lightweight skeleton. They are oviparous. They include, but are not limited to, chickens, ducks, turkeys, geese, and raptors.

The term “mammal-derived component” means a molecule or compound (e.g., a protein, a lipid, or a nucleic acid) obtained from the body of a mammal or a molecule obtained from a fluid or solid produced by a mammal.

The term “avian-derived component” or “bird-derived component” means a molecule or compound (e.g., a protein, a lipid, or a nucleic acid) obtained from the body of an avian/bird or a molecule obtained from a fluid or solid produced by an avian/bird.

The term “lipids” means one or more molecules (e.g., biomolecules) that include a fatty acyl group (e.g., saturated or unsaturated acyl chains). For example, the term lipids includes oils, phospholipids, free fatty acids, phospholipids, monoglycerides, diglycerides, and triglycerides. Additional examples of lipids are known in the art.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” optionally also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

One of ordinary skill in the art would appreciate that the term “gene” may encompass a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The skilled artisan would appreciate that the term “gene” optionally also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

In one embodiment, a gene comprises DNA sequence comprising upstream and downstream regions, as well as the coding region, which comprises exons and any intervening introns of the gene. In some embodiments, upstream and downstream regions comprise non-coding regulatory regions. In some embodiments, upstream and downstream regions comprise regulatory sequences, for example but not limited to promoters, enhancers, and silencers. Non-limiting examples of regulatory sequences include, but are not limited to, AGGA box, TATA box, Inr, DPE, ZmUbi1, PvUbi1, PvUbi2, CaMV, 35S, OsAct1, zE19, E8, TA29, A9, pDJ3S, B33, PAT1, alcA, G-box, ABRE, DRE, and PCNA. Regulatory regions, may in some embodiments, increase or decrease the expression of specific genes within a subject, tissue, cell, or cell line described herein.

In another embodiment, a gene comprises the coding regions of the gene, which comprises exons and any intervening introns of the gene. In another embodiment, a gene comprises its regulatory sequences. In another embodiment, a gene comprises the gene promoter. In another embodiment, a gene comprises its enhancer regions. In another embodiment, a gene comprises 5′ non-coding sequences. In another embodiment, a gene comprises 3′ non-coding sequences.

In one embodiment, the skilled artisan would appreciate that DNA comprises a gene, which may include upstream and downstream sequences, as well as the coding region of the gene. In another embodiment, DNA comprises a cDNA (complementary DNA). One of ordinary skill in the art would appreciate that cDNA may encompass synthetic DNA reverse transcribed from RNA through the action of a reverse transcriptase. The cDNA may be single stranded or double stranded and can include strands that have either or both of a sequence that is substantially identical to a part of the RNA sequence or a complement to a part of the RNA sequence. Further, cDNA may include upstream and downstream regulatory sequences. In still another embodiment, DNA comprises CDS (complete coding sequence). One of ordinary skill in the art would appreciate that CDS may encompass a DNA sequence, which encodes a full-length protein or polypeptide. A CDS typically begins with a start codon (“ATG”) and ends at (or one before) the first in-frame stop codon (“TAA”, “TAG”, or “TGA”). The skilled artisan would recognize that a cDNA, in one embodiment, comprises a CDS.

The terms “polynucleotide”, “polynucleotide sequence”, “nucleic acid sequence”, and “isolated polynucleotide” are used interchangeably herein. These terms encompass nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

The term “RNA interference” or “RNAi” refers to the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

Typically, the term RNAi molecule refers to single- or double-stranded RNA molecules comprising both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “construct” as used herein refers to an artificially assembled or isolated nucleic acid molecule which includes the polynucleotide of interest. In general, a construct may include the polynucleotide or polynucleotides of interest, a marker gene which in some cases can also be a gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

The terms “promoter element,” “promoter,” or “promoter sequence” as used herein, refer to a DNA sequence that is located at the 5′ end (i.e. precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

As used herein, the term an “enhancer” refers to a DNA sequence which can stimulate promoter activity and may be an innate element of the promoter or a heterologous element inserted to enhance the level or tissue-specificity of a promoter.

The term “expression”, as used herein, refers to the production of a functional end-product e.g., an mRNA or a protein.

Down-regulation or inhibition of the gene expression can be effected on the genomic and/or the transcript level using a variety of molecules that interfere with transcription and/or translation (e.g., antisense, siRNA, Ribozyme, or DNAzyme), or on the protein level using, e.g., antagonists, enzymes that cleave the polypeptide, and the like.

The silencing molecule (silencer) can be designed as is known to a person skilled in the art. According to certain embodiments, the silencer comprises a polynucleotide having a nucleic acid sequence substantially complementary to a region of a polynucleotide encoding the nucleic acid sequence targeted. According to certain embodiments, the silencer comprises a guide-RNA pair. According to certain embodiments, the guide-RNA pair is targeted to a 5′-translated region of a polynucleotide encoding the target nucleic acid sequence of interest. According to certain embodiments, multiple guide-RNA pairs target multiple nucleic acid sequences of interest. According to certain embodiments, multiple guide-RNA (gRNA) pairs are encoded by a guide-RNA expression multiarray complex under the control of an independent guide-RNA expression multiarray complex promoter and in an array cleavable by a CRISPR/CAS6 RNA endonuclease. According to certain embodiments, a CRISPR/CAS system for multiple gene targeting is used to construct the multiplex guide-RNA array of multiple guide-RNA pairs targeting the genes of interest.

The term “gene edited organism” refers to an organism comprising at least one cell comprising at least one gene edited by man. The gene editing includes deletion, insertion, silencing, or repression, such as of the “native genome” of the cell. Methods for creating a gene edited organism include techniques such as zinc-finger nucleases (ZFN), transcription activator-like effector nucleases (TALEN), and clustered regularly interspersed short palindromic repeats (CRISPR)/Cas systems.

The term “genetically modified organism” refers to an organism comprising at least one cell genetically modified by man. The genetic modification includes modification of an endogenous gene(s), for example by introducing mutation(s) deletions, insertions, transposable element(s) and the like into an endogenous polynucleotide or gene of interest. Additionally, or alternatively, the genetic modification includes transforming the cell with heterologous polynucleotide. A “genetically modified organism” and a “corresponding unmodified organism” as used herein refer to an organism comprising at least one genetically modified cell and to an organism of the same type or species lacking said modification, respectively.

One of ordinary skill in the art would appreciate that a genetically modified organism may encompass an organism comprising at least one cell genetically modified by man. In some embodiments, the genetic modification includes modification of an endogenous gene(s), for example by introducing mutation(s) deletions, insertions, transposable element(s) and the like into an endogenous polynucleotide or gene of interest. Additionally, or alternatively, in some embodiments, the genetic modification includes transforming at least one organism cell with a heterologous polynucleotide or multiple heterologous polynucleotides. The skilled artisan would appreciate that a genetically modified organism comprising transforming at least one organism cell with a heterologous polynucleotide or multiple heterologous polynucleotides may in certain embodiments be termed a “transgenic organism.”

A skilled artisan would appreciate that a comparison of a “genetically modified organism” to a “corresponding unmodified organism” as used herein encompasses comparing an organism comprising at least one genetically modified cell and to an organism of the same type or species lacking the modification.

The skilled artisan would appreciate that the term “transgenic” when used in reference to an organism as disclosed herein encompasses an organism that contains at least one heterologous transcribable polynucleotide in one or more of its cells. The term “transgenic material” encompasses broadly an organism or a part thereof, including at least one cell, multiple cells or tissues that contain at least one heterologous polynucleotide in at least one of cell. Thus, comparison of a “transgenic organism” and a “corresponding non transgenic organism”, or of a “genetically modified organism comprising at least one cell having altered expression, wherein said organism comprising at least one cell comprising a heterologous transcribable polynucleotide” and a “corresponding unmodified organism” encompasses comparison of the “transgenic organism” or “genetically modified organism” to an organism of the same type lacking said heterologous transcribable polynucleotide. A skilled artisan would appreciate that, in some embodiments, a “transcribable polynucleotide” comprises a polynucleotide that can be transcribed into an RNA molecule by an RNA polymerase.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. In contrast, the term “stable transformation” or “stably transformed” refers to the introduction and integration of one or more exogenous polynucleotides into the genome of a cell. The term “stable transformant” refers to a cell which has stably integrated one or more exogenous polynucleotides into the genomic or organellar DNA. It is to be understood that an organism, tissue, cell line, or cell transformed with the nucleic acids, constructs and/or vectors of the present invention can be transiently as well as stably transformed.

The skilled artisan would appreciate that the term “construct” may encompass an artificially assembled or isolated nucleic acid molecule which includes the polynucleotide of interest. In general, a construct may include the polynucleotide or polynucleotides of interest, a marker gene which in some cases can also be a gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The skilled artisan would appreciate that the term “expression” may encompass the production of a functional end-product e.g., an mRNA or a protein.

As used herein, the singular form “a”, “an” and “the” include plural references unless the context clearly dictates otherwise. For example, the term “a molecule” also includes a plurality of molecules.

The term “about” or “approximately” means within an acceptable error range for the particular value as determined by one of ordinary skill in the art, which will depend in part on how the value is measured or determined, i.e., the limitations of the measurement system. It should be understood that all ranges and quantities described below are approximations and are not intended to limit the invention. Where ranges and numbers are used these can be approximate to include statistical ranges or measurement errors or variation. In some embodiments, for example, measurements could be plus or minus 10%. For example, “about” can mean within 1 or more than 1 standard deviations, per practice in the art. Alternatively, when referring to a measurable value such as an amount, a temporal duration, a concentration, and the like, may encompass variations of ±20% or ±10%, more preferably ±5%, even more preferably ±1%, and still more preferably ±0.1% from the specified value, as such variations are appropriate to perform the disclosed methods.

The terms “comprises”, “comprising”, “includes”, “including”, “having” and their conjugates mean “including but not limited to”.

The term “consisting of” means “including and limited to”.

As used herein, the term “consisting essentially of” means that consisting largely, but not necessarily entirely, of a recited element.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “predominantly” or variations thereof will be understood to mean, for instance, a) in the context of fats the amount of a particular fatty acid composition relative to the total amount of fatty acid composition; b) in the context of protein the amount of a particular protein composition (e.g., β-casein) relative to the total amount of protein composition (e.g., α-, β-, and κ-casein).

The phrase “essentially free of” is used to indicate the indicated component, if present, is present in an amount that does not contribute, or contributes only in a de minimus fashion, to the properties of the composition. In various embodiments, where a composition is essentially free of a particular component, the component is present in less than a functional amount. In various embodiments, the component may be present in trace amounts. Particular limits will vary depending on the nature of the component, but may be, for example, selected from less than 10% by weight, less than 9% by weight, less than 8% by weight, less than 7% by weight, less than 6% by weight, less than 5% by weight, less than 4% by weight, less than 3% by weight, less than 2% by weight, less than 1% by weight, or less than 0.5% by weight.

Unless indicated otherwise, percentage (%) of ingredients refer to total % by weight.

Unless otherwise indicated, and as an example for all sequences described herein under the general format “SEQ ID NO:”, “nucleic acid comprising SEQ ID NO:#” refers to a nucleic acid, at least a portion of which has either (i) the sequence of SEQ ID NO:#, or (ii) a sequence complementary to SEQ ID NO:#. The choice between the two is dictated by the context. For instance, if the nucleic acid is used as a probe, the choice between the two is dictated by the requirement that the probe be complementary to the desired target.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1. A Chimeric Protein Construct and a Method of Producing a Chimeric Antigen Receptor In Vivo

A chimeric protein construct is constructed having a clathrin protein moiety and a non-clathrin protein moiety comprising a chimeric antigen receptor (CAR) comprising an ectodomain comprising an antigen binding domain, a transmembrane domain, and an endodomain comprising an intracellular signaling domain and a costimulatory domain. The ectodomain optionally comprises a transport signal peptide, and the endodomain optionally comprises one or more additional costimulatory domain(s) and/or at least one nuclear factor of activated T cell, B cell, Treg call responsive inducible expression element.

A patient is administered the chimeric protein construct, which taken up by endocytosis into an immune effector cell, leading to its activation. The antigen binding domain of the CAR recognizes the corresponding antigen on a target cell of interest.

### Example 2. A Clathrin Light Chain-Type Chimeric Protein Construct and a Method of Producing a Chimeric Antigen Receptor In Vivo

A chimeric protein construct comprises a chimeric antigen receptor (CAR) comprising an ectodomain comprising an antigen binding domain, a transmembrane domain, and an endodomain comprising an intracellular signaling domain and a costimulatory domain. The CAR is attached to clathrin light chain (Protin-101) connected through a tether or fusion. The CAR ectodomain optionally comprises a transport signal peptide, and the endodomain optionally comprises one or more additional costimulatory domain(s) and/or at least one nuclear factor to activated T cell, B cell, Treg cell-responsive inducible expression element.

A patient is administered the Protin-101-chimeric protein construct to target an immune effector cell (T-cell, B-cell or T-reg cell) specifically targeted by the chimeric construct, leading to their in vivo activation. The antigen binding domain of the CAR recognizes the corresponding antigen on a target cell of interest.

### Example 3. A Clathrin Heavy Chain-Type Chimeric Protein Construct and a Method of Producing a Chimeric Antigen Receptor In Vivo

A chimeric protein construct comprises a chimeric antigen receptor comprising an ectodomain comprising an antigen binding domain, a transmembrane domain, and an endodomain comprising an intracellular signaling domain and a costimulatory domain. The CAR is attached to a clathrin heavy chain connected through a tether or fusion. The CAR ectodomain optionally comprises a transport signal peptide, and the endodomain optionally comprises one or more additional costimulatory domain(s) and/or at least one nuclear factor to activated T cell, B cell, Treg cell responsive inducible expression element.

A patient is administered the Protin-102 (clathrin heavy chain) chimeric protein construct to target an immune effector cell (T-cell, B-cell or T-reg cell) specifically targeted by the chimeric construct, leading to their in vivo activation. The antigen binding domain of the CAR recognizes the corresponding antigen on a target cell of interest.

### Example 4. A Clathrin Light Chain/Heavy Chain-Type Chimeric Protein Construct and a Method of Producing a Chimeric Antigen Receptor In Vivo

A chimeric protein construct comprises a chimeric antigen receptor comprising an ectodomain comprising an antigen binding domain, a transmembrane domain, and an endodomain comprising an intracellular signaling domain and a costimulatory domain. The CAR is attached to a clathrin light chain and heavy chain combination connected through a tether or fusion. The CAR ectodomain optionally comprises a transport signal peptide, and the endodomain optionally comprises one or more additional costimulatory domain(s) and/or at least one nuclear factor to activated T cell, B cell, Treg cell-responsive inducible expression element.

The clathrin protein moiety (clathrin light chain and clathrin heavy chain) is optionally self-assembled. Optionally, the clathrin protein moiety comprises multiple clathrin light and heavy chains.

A patient is administered the Protin-101/102 (clathrin light chain and heavy chain combination) chimeric protein construct to target an immune effector cell (T-cell, B-cell or T-reg cell) specifically targeted by the chimeric construct, leading to their in vivo activation. The antigen binding domain of the CAR recognizes the corresponding antigen on a target cell of interest.

### Example 5. A Clathrin Light Chain/Heavy Chain-Type Chimeric Protein Construct with an Antibody and a Method of Producing a Chimeric Antigen Receptor In Vivo

A chimeric protein construct comprises a chimeric antigen receptor comprising an ectodomain comprising an antigen binding domain, a transmembrane domain, and an endodomain comprising an intracellular signaling domain and a costimulatory domain. The CAR is attached to clathrin light chain and heavy chain combination connected through a tether or fusion. The CAR ectodomain optionally comprises a transport signal peptide, and the endodomain optionally comprises one or more additional costimulatory domain(s) and/or at least one nuclear factor to activated T cell, B cell, Treg cell-responsive inducible expression element. This combo construct is attached to an additional non-chimeric antibody targeting specific tumor type.

A patient is administered the Protin-101/102 (clathrin light chain and heavy chain combination)-chimeric protein construct to target an immune effector cell (T-cell, B-cell or T-reg cell) specifically targeted by the chimeric construct, leading to their in vivo activation for a specific tumor type. The antigen binding domain of the CAR recognizes the corresponding antigen on a target cell of interest.

### Example 6. Clathrin Cage (Light Chain, Heavy Chain, and/or Light Chain/Heavy Chain Combination Cages) Chimeric Protein Constructs and a Method of Producing a Chimeric Antigen Receptor In Vivo

A chimeric protein construct comprises a chimeric antigen receptor comprising an ectodomain comprising an antigen binding domain, a transmembrane domain, and an endodomain comprising an intracellular signaling domain and a costimulatory domain. The CAR is attached to a clathrin cage any of the following forms: a) clathrin light chain cage, b) clathrin heavy chain cage, and/or c) clathrin light/heavy chain combination cage, each of which is connected through a tether or fusion. The CAR ectodomain optionally comprises a transport signal peptide, and the endodomain optionally comprises one or more additional costimulatory domain(s) and/or at least one nuclear factor to activated T cell, B cell, Treg cell-responsive inducible expression element. This combo construct is attached to an additional non-chimeric antibody targeting a specific tumor type.

A patient is administered the clathrin cage (e.g., Protin-101/102 [clathrin light chain and heavy chain combination])-chimeric protein construct to target an immune effector cell (T-cell, B-cell or T-reg cell) specifically targeted by the chimeric construct, leading to their in vivo activation for a specific tumor type. The antigen binding domain of the CAR recognizes the corresponding antigen on a target cell of interest.

### Example 7. A Clathrin Cage Chimeric Protein Construct with a Payload and a Method of Producing a Chimeric Antigen Receptor In Vivo

A chimeric protein construct comprises a chimeric antigen receptor with an ectodomain comprising an antigen binding domain, a transmembrane domain, and an endodomain comprising an intracellular signaling domain and a costimulatory domain. The CAR is attached to clathrin cage containing a payload to treat cancer where the cage is of any of the following forms: a) clathrin light chain cage, b) clathrin heavy chain cage, or c) clathrin light/heavy chain combination cage. The entire construct is connected through a tether or fusion. The CAR ectodomain optionally comprises a transport signal peptide, and the endodomain optionally comprises one or more additional costimulatory domain(s) and/or at least one nuclear factor to activated T cell, B cell, Treg cell-responsive inducible expression element. This combination construct is attached to an additional non-chimeric antibody targeting specific tumor type.

A patient is administered the (Protin-101/102 [clathrin light chain and heavy chain combination])-chimeric protein construct to target an immune effector cell (T-cell, B-cell or T-reg cell) specifically targeted by the chimeric construct, leading to their in vivo activation for a specific tumor type. The antigen binding domain of the CAR recognizes the corresponding antigen on a target cell of interest.

### Example 8. A Method of Producing a Chimeric Antigen Receptor in a T-Cell In Vivo

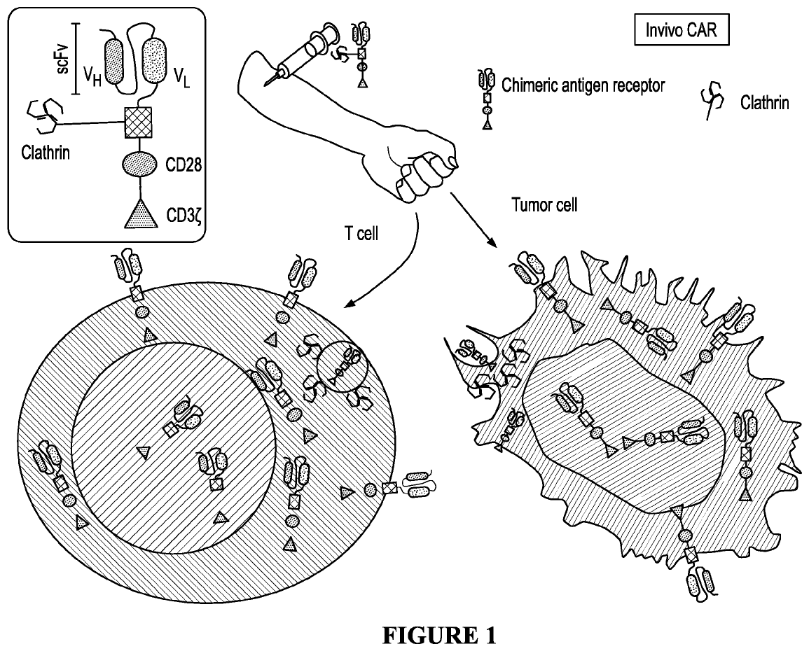

A patient with a tumor or other medical disease or condition to be treated is administered, via injection, a chimeric protein construct comprising a clathrin and chimeric antigen receptor (CAR) of the previous examples, where the CAR contains an ectodomain having a variable scFv; a transmembrane domain; and an endodomain comprising CD28 and CD3-zeta (CD3-ζ), that activates the T-cell, B-cell, Treg CAR receptor (FIG. 1).

### Example 9. A Method of Producing a Chimeric Antigen Receptor in a Tumor Cell In Vivo

A patient with a tumor is administered, via injection, a chimeric protein construct comprising a clathrin and a chimeric antigen receptor (CAR) comprising an ectodomain having a variable scFv; a transmembrane domain; and an endodomain comprising CD28 and CD3-zeta (CD3ζ), the clathrin in this example being attached via a linker (FIG. 1,) that activates the T or B-CAR receptor (FIG. 1). The procedure is also used to create CAR-Treg cells.

### Example 10. A Chimeric Clathrin Light Chain and CAR Specific for Programmed Death-Ligand 1 (PD-1L)

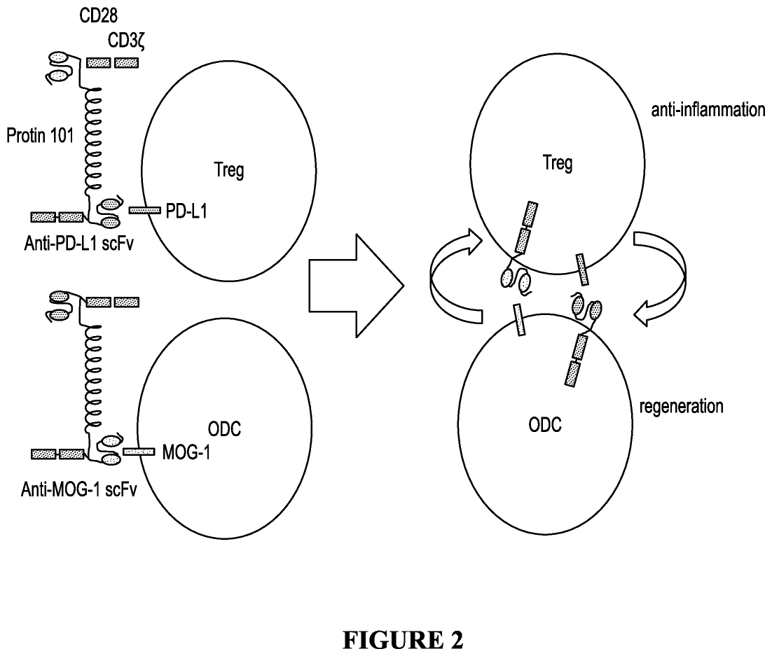

A chimeric clathrin light chain (Protin-101) molecule harboring a CAR on regulatory T cells (T regulatory cells [Tregs]) and specific for myelin oligodendrocyte glycoprotein-1 (MOG-1) (on oligodendrocytes [ODC]) is constructed as described herein (FIG. 2). Binding to cell surface facilitates the endocytosis of molecules into ODC. This results in Tregs expressing anti-MOG-1 and the subsequent interaction between Tregs and oligodeoxynucleotides (ODN).

### Example 11. A Chimeric Clathrin Light Chain and CAR Specific for Programmed Death-Ligand 1 (PD-1L)

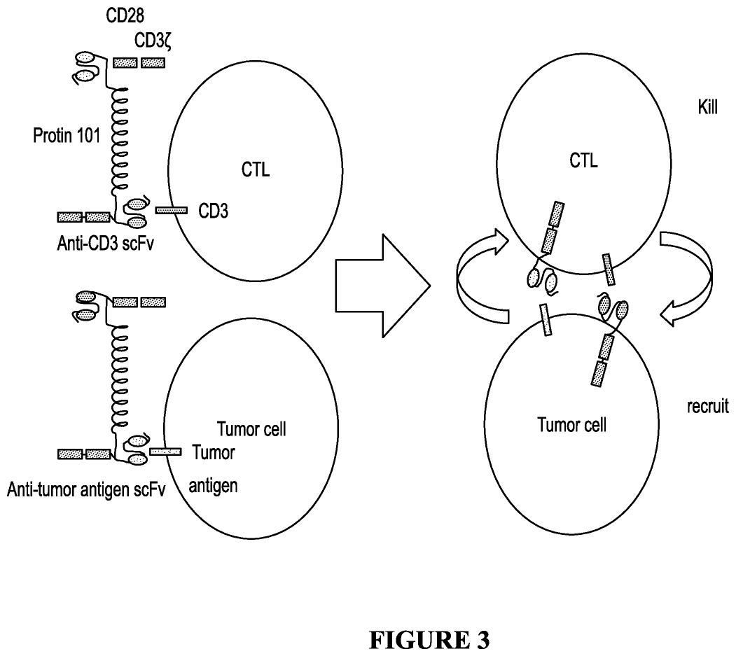

A chimeric clathrin light chain (Protin-101) molecule harboring a CAR specific for CD3 (on cytotoxic T cells [CTL]) and a CAR-specific for a tumor antigen protein (on tumor cells) is constructed as described herein (FIG. 3). Binding of anti-CD3 scFv to cell surface CD3 facilitates the endocytosis of molecules into CTL, and the binding CAR results in subsequent interaction between CTL and tumor cells.

### Example 12. Assembly and Disassembly of Clathrin Cages in Buffer

In first experiment, a clathrin cage was constructed by mixing the proteins with 2-(N-morpholino) ethanesulfonic acid (MES) buffer (pH 6.2) at time zero and measuring absorbance at 320 nm as a function of time. As shown in FIG. 4A, the absorbance at 320 increased with time, which confirms the clathrin cage formation. Representative transmission electron microscopy (TEM) images of cage assembly of the mixture of Protin-101-light chains and Protin-102-heavy chains show the presence of clathrin cages of different sizes (FIG. 4B).

To assess the stability of clathrin cages at a higher pH, the protein solution was mixed with MES buffer (pH 6.2) at time zero (initially as above), and the absorbance was read at 320 nm, as shown in the graph (FIG. 4C). At 500 seconds, disassembly of the clathrin cages was induced by addition of 1M tris(hydroxymethyl)aminomethane-HCl (Tris-HCl) buffer (pH 9) after an overall increase of the buffer pH to 6.5 (FIG. 4C). By increasing the pH, the cages disassembled, and so the absorbance at 320 nm decreased (FIG. 4C). A representative TEM image of a clathrin cage, disassembled after the increase of the pH is shown (FIG. 4D).

### Example 13. Trafficking of Clathrin Cages in Mice

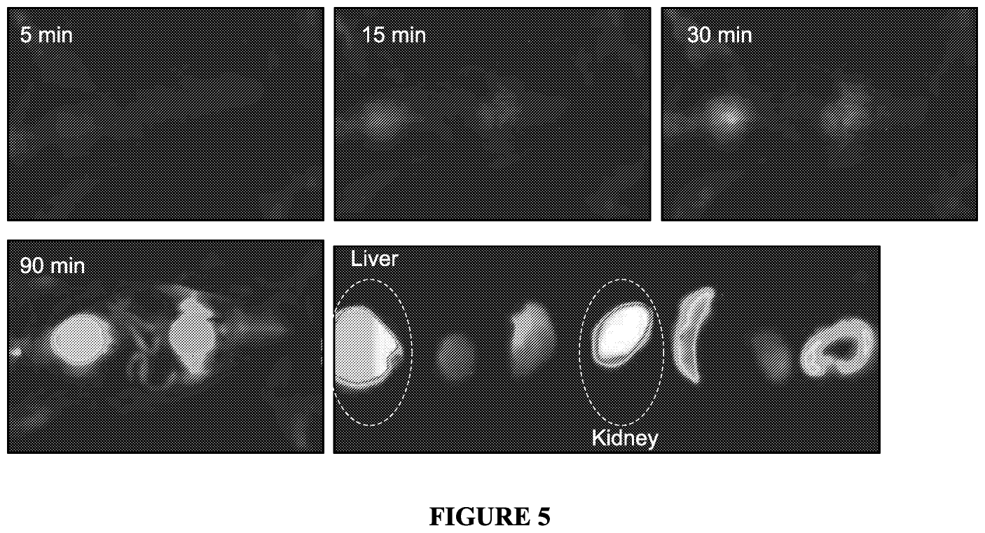

FIG. 5 shows a series of sequential photographic images depicting the trafficking of clathrin cages in a C57BL/6 mouse over the course of 90 minutes. The clathrin light chains were labeled with IR-800 fluorescent dye, then mixed with clathrin heavy chains, and the addition of MES buffer (pH 6.2) was used to synthesize clathrin cages. After concentrating the clathrin cages, 100 microliters (μl) of clathrin concentrate was injected into a C57BL/6 (B6) mouse, and live imaging was performed for 90 minutes (photographs taken at 5 min, 15 min and 30 min [top] and at 90 min [bottom]). The highest level of signal was observed in the liver and the kidneys (red ovals), followed by the spleen. The organs shown are, left to right: liver, heart, lung, kidney, spleen, pancreas, and intestine.

### Example 14. Comparison of Biodistribution and Leakage of Fluorescent Dye, Either Alone or Contained in Clathrin Cages, when Injected into Mice

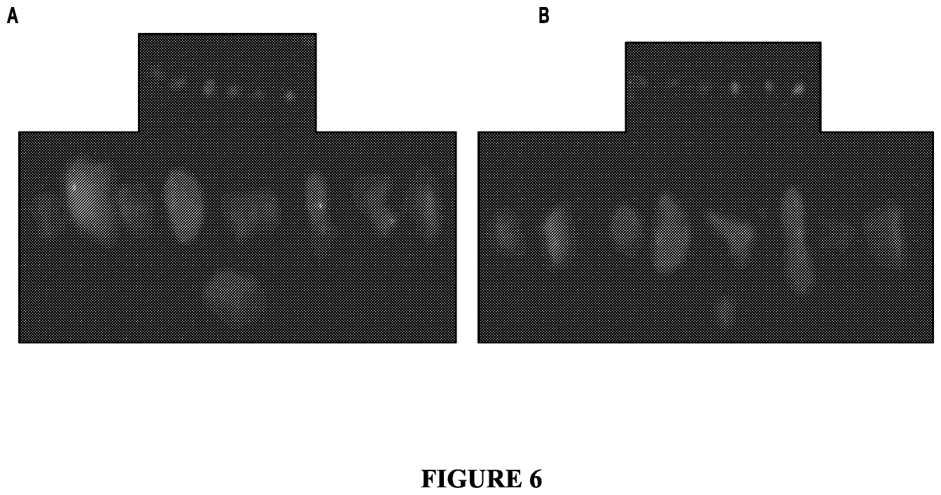

The physical loading of IR800-dye in a clathrin cage was studied as a function of leakage. In this experiment, IR800 fluorescent dye was loaded into clathrin cages and injected into C57BL/6 (B6) mice. As a control, IR800 fluorescent dye was injected directly into C57BL/6 control mice. Mice were sacrificed 24 hours post-injection, and the major organs and lymph nodes were imaged (FIGS. 6A-6B).

FIGS. 6A-6B are photographic images depicting the biodistribution of free IR800 fluorescent dye alone (FIG. 6A) in comparison to the biodistribution of clathrin cages loaded with IR800 fluorescent dye (FIG. 6B). As it is shown in FIGS. 6A-6B, no signal was observed, confirming the dye leaking from cages and clearing through the kidneys in 24 hours.

### Example 15. Conjugation of Anti-PD-1 by Protin-101 (Prot101) and Purification Thereof

Conjugation of anti-PD-1 (IgG2; molecular weight approximately 55 kDa) by Protin-101 (Prot101) was performed. Prot101 was labeled by ALEXA FLUOR® 594 via maleimide chemistry. (ALEXA FLUOR® 594 is an anti-mouse/human CD45R/B220 antibody. The CD45 isoform B220 identifies select subsets of human B cells and B-cell lymphoproliferative disorders.) The labeled Prot101 was activated by EDC/sulfo-NHS for 15 minutes, followed by quenching of excessive EDC by 2-mercaptoethanol (beta-mercaptoethanol) for 10 minutes. Anti-PD-1 antibody was added and conjugated to the activated Protin-101 overnight at 4° C.

The anti-PD-1/Prot101 conjugate was purified by 50 kDa centrifugal membrane filtration (10,000 rpm for 5 mins; concentration factor of 10).

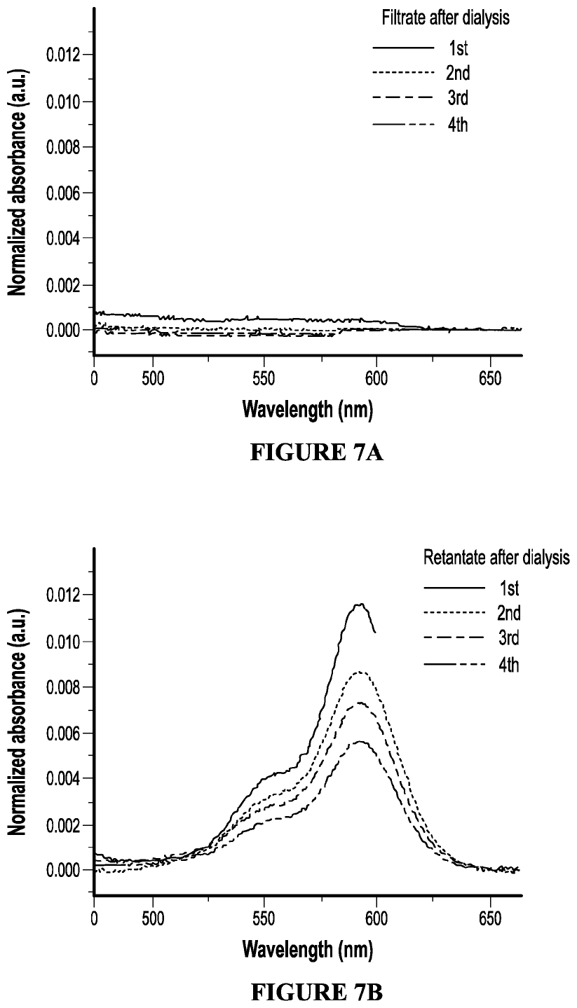

Absorbance from ALEXA FLUOR® 594 was measured in post-dialyses (1st, 2nd, 3rd, and 4th) filtrates and retentates. The filtrates showed no significant absorbance from ALEXA FLUOR® 594 (Alexa594) (FIG. 7A), indicating that the conjugation yield reached 100%. With respect to the rententates, decreasing absorbance from ALEXA FLUOR® 594 with respect to increasing rounds of dialyses was observed (FIG. 7B).

Alternatively, desalting is used for the purification or succinimidyl 4[N-maleimidomethyl]cyclohexane-1-carboxylate) (SMCC)/sulfosuccinimidyl 4[N-maleimidomethyl] cyclohexane-1-carboxylate (Sulfo-SMCC) chemistry is used for the conjugation.

### Example 16. Trafficking of Protin-101 (Prot101) in a B6 Mouse

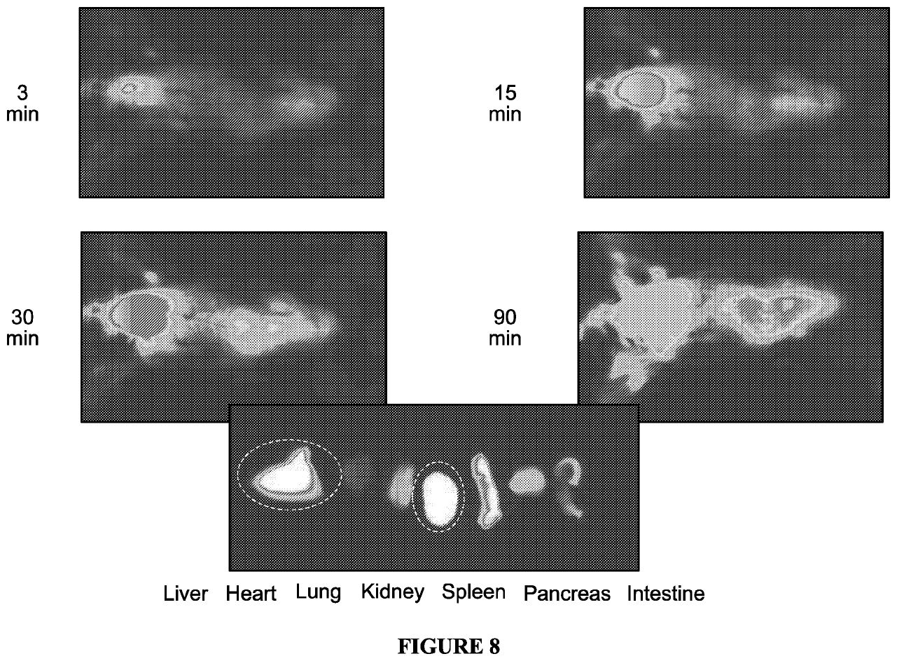

Trafficking of clathrin light chains (Protin-101) was observed in a B6 mouse over the course of 90 minutes. The clathrin light chains were labeled with IR-800 fluorescent dye. The clathrin light chains were then injected into a C57BL/6 (B6) mouse, and live imaging was performed for 90 minutes (photographs taken at 3 min and 15 min [FIG. 8, top] and at 30 min and 90 min [FIG. 8, middle]). The highest level of signal was observed in the liver and the kidney (indicated by the dashed red ovals), followed by the spleen. The organs shown are, left to right: liver, heart, lung, kidney, spleen, pancreas, and intestine (FIG. 8, bottom).

### Example 17. Trafficking of Protin-102 (Prot102) in a B6 Mouse

Trafficking of clathrin heavy chains (Protin-102) was observed in a B6 mouse. The clathrin heavy chains were labeled with CF680 dye, a cyanine-based dye that reacts with alkyne and is commonly used for labeling antibodies. The clathrin heavy chains were then injected into a C57BL/6 (B6) mouse, and imaging was performed 24 hours post-injection.

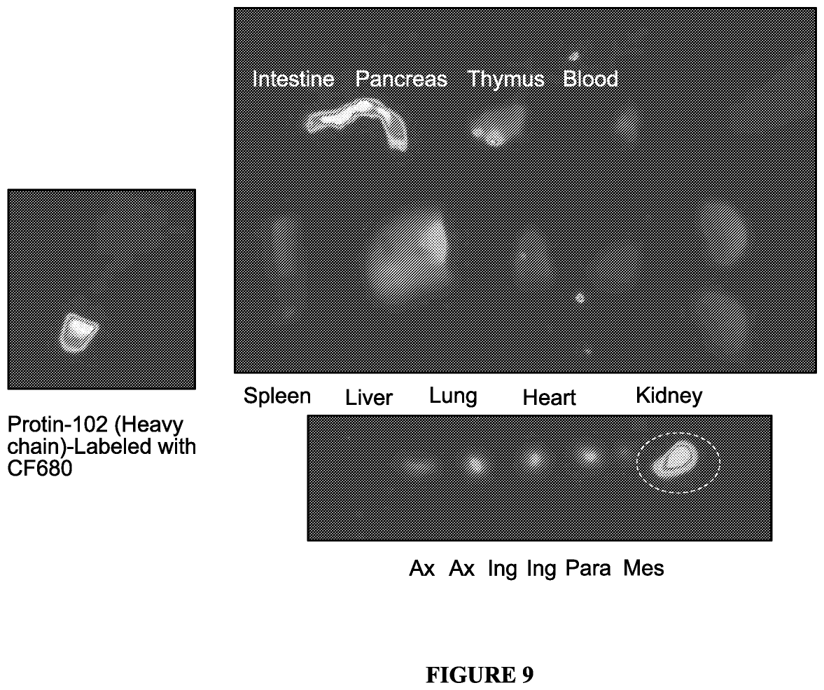

The results are shown in FIG. 9. On the left, the photographic image shows ex vivo images of the concentration in the mesenteric lymph nodes (left). The highest level of signal was observed in the liver (top right). Further review showed the concentration in the mesenteric lymph nodes (Mes) as indicated by the oval red dashed (bottom right), as compared with the axillary (Ax) and inguinal (Ing) lymph nodes.

### Example 18. Trafficking of Protin-101 (Prot101) Post-Implantation in a Mouse with Kidney Tumors

A B6 mouse was implanted with tumors in the kidneys. Eight days post-implantation, the mouse was injected with Protin-101 (clathrin light chains), and histological studies were subsequently performed on the left (left) and right (right) kidneys. The kidneys were stained.

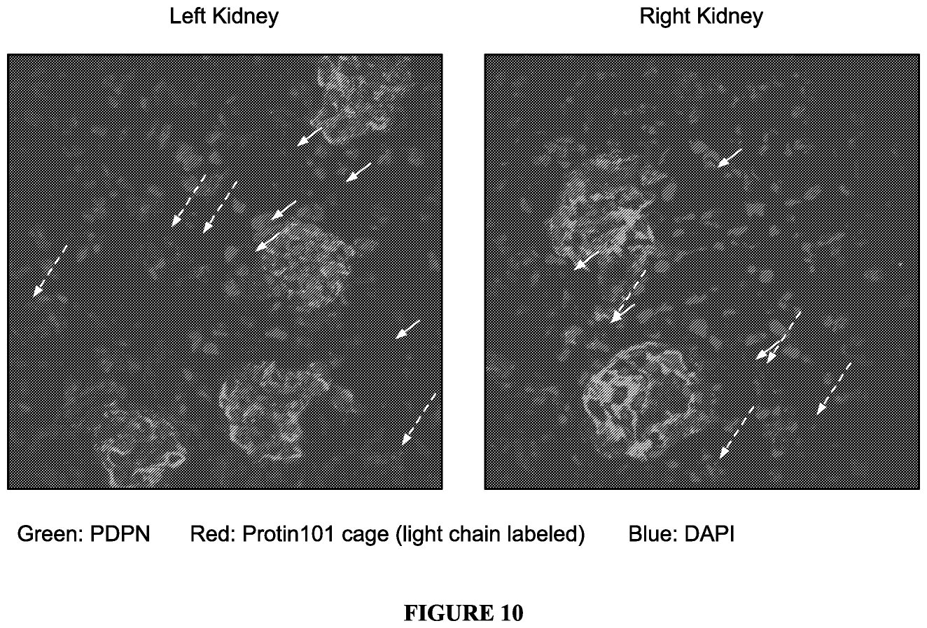

As shown in FIG. 10, the green regions indicate podoplanin (PDPN). PDPN is a mucin-type protein that is well-conserved between species and is generally receptive to detection via immunofluorescent staining. It is a specific lymphatic vessel marker that can be used as a diagnostic marker for some types of cancers.) The blue regions are DNA stained with 4′,6-diamidino-2-phenylindole (DAPI; IUPAC 2-(4-amidinophenyl)-1H-indole-6-carboxamidine), a fluorescent stain that binds strongly to adenine-thymine rich regions in DNA. The smaller red spots are Protin-101 (light chain) cages (Prot 101), and blue are directed to DAPI of DNA of kidney cancer.

### Example 19. Trafficking of Protin-101 (Prot101) to Mesenteric Lymph Nodes (MLN) in a Mouse

Trafficking of Protin-101 (clathrin light chains) to mesenteric lymph nodes (MLN) was observed in a B6 mouse injected with Protin-101.

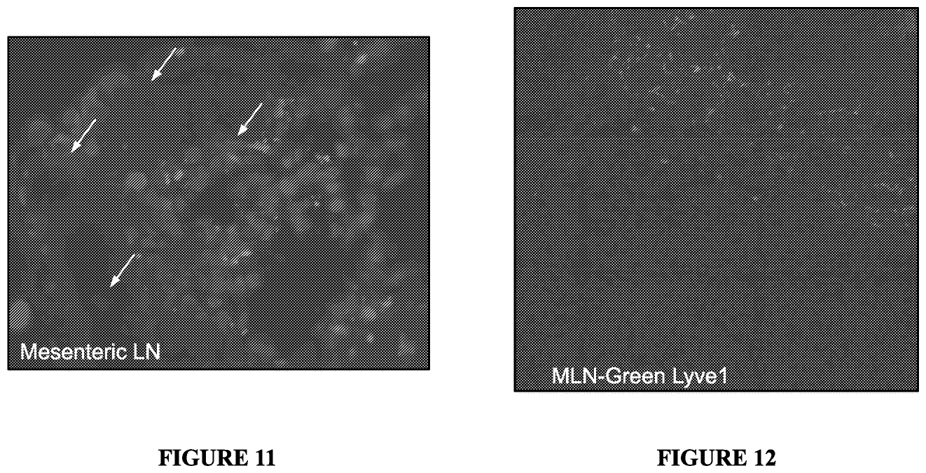

FIG. 11 shows a photographic image depicting a histological examination of MLN and showing trafficking of Protin-101 (Prot 101) (red spots, orange arrows) to the MLN from the B6 mouse.

### Example 20. Trafficking of Protin-101 (Prot101) to Mesenteric Lymph Nodes (MLN) in a Mouse

Trafficking of Protin-101 (clathrin light chains) to mesenteric lymph nodes (MLN) was observed in a B6 mouse injected with Protin-101. The mouse was injected with Protin-101 (clathrin light chains), and histological examination of the MLN was performed.

As shown in FIG. 12, histological examination of mesenteric lymph nodes (MLN) in the injected mouse demonstrated trafficking of Protin-101 (Prot101) (red spots). Green staining is specific for lymphatic vessel endothelial hyaluronan receptor 1 (LYVE1), also known as extracellular link domain containing 1 (XLKD1). LYVE1 is a Link domain-containing hyaladherin, a protein capable of binding to hyaluronic acid (HA), homologous to CD44, the main HA receptor.

### Example 21. Trafficking of Clathrin Light Chain Cages to Post-Implantation in a Mouse with Kidney Tumors

A B6 mouse was implanted with tumors in the kidneys. Eight days post-implantation, the mouse was injected with Protin-101 (clathrin light chains), and histological studies were subsequently performed on the left kidney. The kidney was stained.

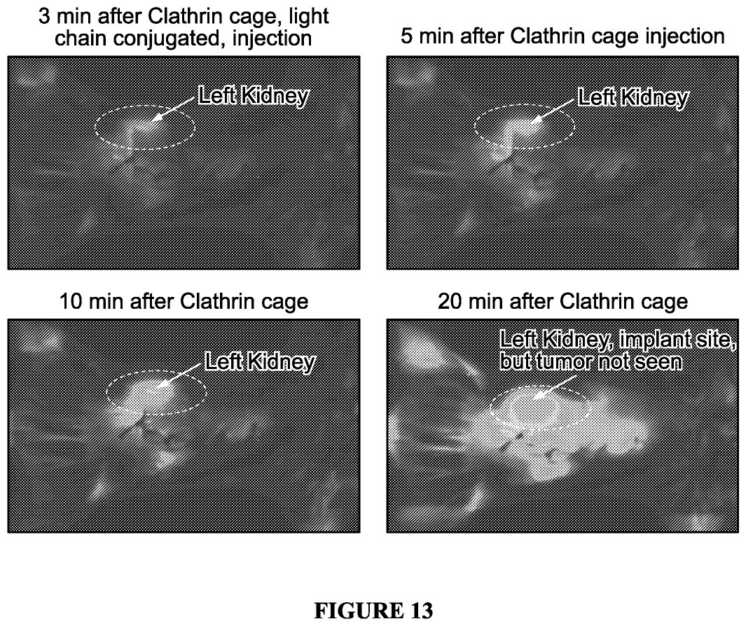

As shown in FIG. 13, the trafficking of clathrin light chain cages in a B6 mouse was observed over the course of 20 minutes. The clathrin light chain cages were then injected into a C57BL/6 (B6) mouse 8 days after tumor implantation in the left kidney, and live imaging was performed for 20 minutes (photographs taken at 3 min and 5 min [FIG. 13, top], and at 10 min and 20 min [FIG. 13, bottom] following injection). The highest level of signal was observed at 20 minutes following administration.

### Example 22. Dose Conversion Analysis of TAXOL®

To perform studies of mice using TAXOL® (paclitaxel), a dose conversion analysis was necessary to determine dosing levels comparable to those used in humans.

According to the U.S. Food & Drug Administration (FDA) label for TAXOL® (paclitaxel) Injection (Patient Information Included) (Ref. ID No. 2939751; BRISTOL-MYERS SQUIBB™) (“TAXOL® FDA Label”), the manufacturers studied pharmacokinetic parameters of paclitaxel following 3- and 24-hour infusions of TAXOL® at dose levels of 135 mg/m2 and 175 mg/m2 in a Phase 3 randomized study of ovarian cancer patients with the results shown in TABLE 2 (TAXOL® FDA Label; Table 1, p. 3).

TABLE 2
Summary of Pharmacokinetic Parameters - Mean Values.
SUMMARY OF PHARMACOKINETIC PARAMETERS-MEAN VALUES

| Dose | Infusion | N | Cₘₐₓ | AUC_{(0-∞)} | T-HALF | CL [symbol] |
|---|---|---|---|---|---|---|
| (mg/m²) | Duration (h) | (patients) | (ng/mL) | (ng*h/mL) | (h) | (L/h/m²) |
| 135 | 24 | 2 | 195 | 6300 | 52.7 | 21.7 |
| 175 | 24 | 4 | 365 | 7993 | 15.7 | 23.8 |
| 135 | 3 | 7 | 2170 | 7952 | 13.1 | 17.7 |
| 175 | 3 | 5 | 3650 | 15007 | 20.2 | 12.2 |

Cₘₐₓ = Maximum plasma concentration
AUC_{(0-∞)} = Area under the plasma concentration-time curve from time 0 to infinity
CL [symbol] = Total body clearance [symbol]
indicates data missing or illegible when filed

The manufacturer studied efficacy as a function of response rates, median survival and median time to progression, with the results shown in TABLE 3 (TAXOL® FDA Label; Table 3, p. 8).

TABLE 3
Efficacy in the Phase 3 Second-Line-Ovarian Carcinoma Study.
EFFICACY IN THE PHASE 3 SECOND-LINE OVARIAN CARCINOMA STUDY

| | 175/3 | 175/4 | 155/3 | 135/24 |
|---|---|---|---|---|
| | (u = 96) | (u - 106) | (u = 99) | (u = 106) |
| Response | | | | |
| rate (percent) | 14.6 | 21.7 | 15.2 | 13.2 |
| 95% Confidence Interval | (8.5-23.6) | (145-31.0) | (9.0-24.1) | (7.7-21.5) |
| Time to Progression | | | | |
| median (months) | 4.4 | 4.2 | 3.1 | 2.8 |
| 95% Confidence Interval | (3.0-5.6) | (3.5-5.1) | (2.8-4.2) | (1.9-4.0) |
| Survival | | | | |
| median (months) | 11.5 | 11.8 | 13.1 | 10.7 |
| 95% Confidence Interval | (8.4-14.4) | (8.9-14.6) | (9.1-14.6) | (8.1-13.6) |

The manufacturer also studied efficacy after failure of initial chemotherapy or within six months of adjuvant chemotherapy in the treatment of breast cancer, with the results shown in TABLE 4 (TAXOL® FDA Label; Table 5, p. 16).

TABLE 4
Efficacy in Breast Cancer After Failure of Initial
Chemotherapy or Within 6
Months of Adjuvant Chemotherapy.
EFFICACY IN BREAST CANCER AFTER FAILURE OF
INITIAL CHEMOTHERAPY OR WITHIN 6
MONTHS OF ADJUVANT CHEMOTHERAPY

| | 175/3 | | 135/3 |
|---|---|---|---|
| | (n = 235) | | (n = 236) |
| Response | | | |
| rate (percent) | 28 | | 22 |
| 95% Confidence Interval | | 0.135 | |
| Time to Progression | | | |
| median (months) | 4.2 | | 3.0 |
| 95% Confidence Interval | | 0.027 | |
| Survival | | | |
| median (months) | 11.7 | | 10.5 |
| 95% Confidence Interval | | 0.321 | |

According to the manufacturer, depending on the type of cancer and previous treatment thereof, the doses range between 135 mg/m2 (over 3-24 hours) to 175 mg/m2 (over 3 hours) (TAXOL® FDA Label, pp. 44-47).

As a result, it was necessary to calculate the equivalent animal dose. For purposes of the present calculation, the human dose of TAXOL® used for comparison was 175 mg/m2.

Per U.S. FDA guidance, the conversion of 175 mg/m2 to mg/kg was: 175 mg/m2 divided by a factor of 37 m2/kg (Km ratio), resulting in a human TAXOL® dose of 4.7297 mg/kg in humans. The human equivalent dose (HED) was calculated according to FDA guidance as shown in TABLE 5 (FDA Draft Guidelines; see also).

TABLE 5
Human Equivalent Dose Calculation.

| | Reference | Working | Body | To convert dose | To convert animal dose in mg/kg | |
|---|---|---|---|---|---|---|
| | body | weight | surface | in mg/kg to | to HED in mg/kg, either | |
| | weight | range | area | dose in mg/m², | Divide animal | Multiply animal |
| Species | (kg) | (kg) | (m²) | multiply by K [symbol] | dose by | dose by |
| Human | 60 | — | 1.62 | 37 | — | — |
| Mouse | 0.02 | 0.01 [symbol] -0.034 | 0.007 | 3 | 12.3 | 0.081 |
| Hamster | 0.08 | 0.0047-0.157 | 0.016 | 5 | 7.4 | 0.135 |
| Rat | 0.15 | 0.08-0.27 | 0.025 | 6 | 6.2 | 0.162 |
| Ferret | 0.30 | 0.16-0.54 | 0.043 | 7 | 5.3 | 0.18 [symbol] |
| Guinea pig | 0.40 | 0.208-0.700 | 0.05 | 8 | 4.6 | 0.216 |
| Rabbit | 1.8 | 0.90-3.0 | 0.15 | 12 | 3.1 | 0.324 |
| Dog | 10 | 5-17 | 0.50 | 20 | 1.8 | 0.541 |
| Monkeys (rhesus) | 3 | 1.4-4.0 | 0.25 | 12 | 3.1 | 0.324 |
| Marmoset | 0.35 | 0.14-0.72 | 0.06 | 6 | 6.2 | 0.1 [symbol] 2 |
| Squirrel monkey | 0.60 | 0.29-0.97 | 0.08 | 7 | 5.3 | 0.18 [symbol] |
| Baboon | 12 | 7-23 | 0.60 | 20 | 1.8 | 0.541 |
| Micro pig | 20 | 10-33 | 0.74 | 27 | 1.4 | 0.730 |
| Mini pig | 40 | 25-84 | 1.14 | 35 | 1.1 | 0.946 |

[symbol]

Data obtained from FDA draft guidelines. [symbol]
FDA: Food and Drug Administration, HED: Human equivalent dose [symbol]
indicates data missing or illegible when filed

Here, the animal dose for the present studies was 8 microgram/kg (μg/kg), equaling 0.008 mg/kg. The HED (mg/kg/) was equal to the animal dose (0.008 mg/kg)×0.081=0.000648 mg/kg HED. As a comparison, the 0.000648 mg/kg HED used in the study is only 0.014% of the 4.7297 mg/kg human dose recited in the TAXOL® FDA Label.

### Example 23. Therapeutic Studies of Protin-101-TAXOL® Conjugates (PTCs)

Therapeutic effects of Prot101-TAXOL® conjugates (PTCs; Protin-101-TAXOL®; clathrin light chain-TAXOL® conjugates) were studied in a xenograft BALB/c mouse model bearing 4T1 murine breast cancer, which is highly aggressive and refractory to many chemotherapeutics.

### Materials & Methods

Synthesis of 2′-Glutaryl TAXOL® and Conjugation to Prot101

Glutaric anhydride (100 mg, SIGMA-ALDRICH™) and TAXOL® (33 mg, LC LABORATORIES™) were prepared in a 4 mL vial dried under high vacuum for 24 hrs and dissolved in 1 mL of pyridine. The solution was stirred at room temperature under Ar atmosphere for 2 hrs. The mixture was diluted with 300 μL of methanol and 5 μL of solution was injected into LC/MS (AGILENT 1200™, USA) with a gradient reversed phase system (10% to 100% ACN/H2O with 0.1% formic acid for 20 min) using PHENOMENEX LUNA™ 5 μm C18 column (100×4.6 mm, flow rate 0.7 mL/min, ultraviolet [UV] 250 nm detection). The product was detected at 17.3 min and molecular weight was confirmed as 890 under the ESIMS analysis ([M-H]− m/z at 889]). 2′-Glutaryl TAXOL® was purified by reversed phase high performance liquid chromatography (HPLC) (PHENOMENEX LUNA™ 5 μm C18 250×10.0 mm, flow rate 2 mL/min, UV 600 nm detection) with a gradient solvent system (15% to 75% ACN/H2O with 0.1% formic acid for 40 min). The product was eluted at a retention time of 16.4 min under the HPLC conditions. Reaction yield of 2′ glutaryl TAXOL® was 98.1%, which was extracted by the integration of chromatographic peaks from the product and starting TAXOL®. 2′-Glutaryl TAXOL® dissolved in dimethyl sulfoxide (DMSO) was confirmed by 1H nuclear magnetic resonance (NMR), 13C NMR, correlation spectroscopy (COSY), and heteronuclear single quantum coherence (HSQC) spectra. A carboxylic group on 2′-glutaryl TAXOL® (20 mg) was activated with N,N′-carbonyldiimidazole (CDI, 200 mg, SIGMA-ALDRICH™) for 30 min at 45° C. in anhydrous DMSO (THERMO SCIENTIFIC FISHER™). Prot101 dissolved in phosphate buffered saline (PBS, pH 7.4, CORNING™) was mixed with the activated 2′-glutaryl TAXOL® at room temperature for 2 hrs (1:2 molar ratio of Prot101 to TAXOL®, final solution; approximately 1 mL in 10% DMSO). PTCs were purified by a centrifugal filter (AMICON®, 10 kD molecular weight cut-off [MWCO], SIGMA-ALDRICH™) at 10,000 rpm for 15 min with 3 times.

Ex Vivo Biodistribution of PTC

OREGON GREEN 488™ TAXOL® (TAXOL®*; λex=496 nm and λem=524 nm; THERMO FISHER SCIENTIFIC™) was reacted with glutaric anhydride. The resulting 2′-glutaryl TAXOL®* was activated with CDI and conjugated to Prot101. The detailed reaction condition was identical with the preparation of PTCs. Animal studies were approved and conducted according to the Institutional Animal Care and Use Committee of Brigham and Women's Hospital, Boston, Mass. For tissue biodistribution studies, C57BL/6 BALB/c (JAX #000651; female; 7-8 weeks; n=3) bearing 4T1 (100,000 cells; day 14 post-implant) were anesthetized with isoflurane and received a single intravenous injection of Prot101-TAXOL®* conjugate (2.5 mg/kg). 1, 2, 7, 24, and 72 hrs following injection, the mice were euthanized, and their major organs were harvested and imaged by using a UVP iBOX® EXPLORER™ Imaging Microscope (UVP; ANALYTIK JENA™) equipped with a 500 nm band-pass excitation filter and an 530 nm band-pass emission filter. Mean fluorescence intensity (MFI) for each organ was extracted by ImageJ (National Institutes of Health; Bethesda, Md.; https://imagej.nih.gov/ij/) with constant brightness values for each. All organ MFIs were subtracted by organ fluorescence of non-treated mice.

Therapeutic Studies in Tumor-Bearing Mice

BALB/c (female; 7-8 weeks) were subcutaneously implanted by 4T1 (100,000 cells; ATCC® CRL-2539™) on left 4th mammary glands. C57BL/6 (female; 7-8 weeks) were subcutaneously inoculated by B16 melanoma cells (100,000 cells; ATCC® CRL-6322™) or Lewis lung carcinoma line 1 cells (LLC1) (100,000 cells; ATCC® CRL-1642™) on right rear flanks. When the tumor size reached approximately 100 mm3, mice were randomly divided into three groups (n=6). All groups were treated intravenously with samples; the first group was injected with PBS (control), the second group with free TAXOL® (8 μg/kg), and final group with Prot101-TAXOL® conjugates (PTCs; TAXOL®=8 μg/kg; Prot101=240 μg/kg). The injection schedule was twice a week for two weeks. The tumor size and body weight of the mice were monitored during the treatment course. The length (l) and width (w) of the tumor were measured by a digital vernier caliper, and tumor volume (V) was defined as: V=1×w2/V=l×w2/2. The tumor growth inhibition (TGI) was estimated by a following equation: TGI (%)=(Vc−Vt)/(Vc−Vi)×100, where Vc is the volume of the control tumor at the end of the study, Vt is the volume of drug-treated tumor at the end of the study, and Vi is the volume of tumor at the initial treatment.

Before the diameter of tumor reached approximately 20 mm, the mice were euthanized, and lungs, livers, kidneys, hearts, pancreas, spleen, tumor, non-drain inguinal lymph nodes (far from tumor), and drain inguinal lymph nodes (located near tumor) were harvested and embedded in optimum cutting temperature (OCT) compound (TISSUE TEK™; SAKURA FINETEK™; Torrance, Calif.).

### Results & Discussion

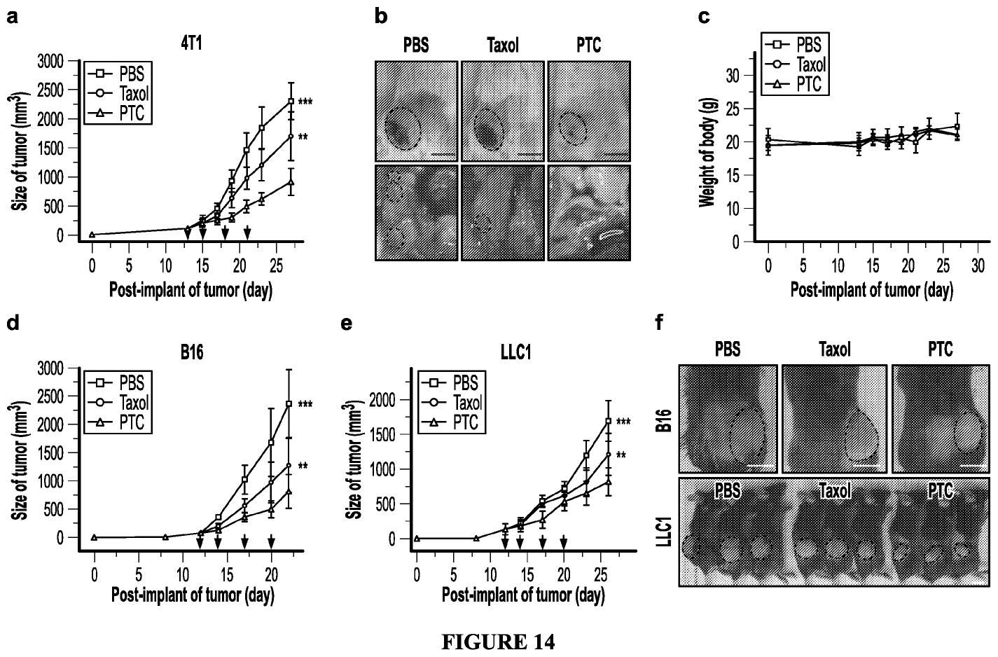

On day 13 post-implant of 1×105 4T1 cells when tumor volume reaches ˜100 mm3, the mice were randomly divided into 3 groups, based on the size of tumor, and were intravenously administrated by the following formulations 2 times per week for 2 weeks: PBS, free TAXOL®, and PTC. The group of PTC dramatically reduced tumor progression, compared with other treatment groups during the course of treatment (total dose of TAXOL®=32 μg/kg, FIG. 14A). The final volume of the tumor (day 27 post-implant) was significantly lower in mice treated with PTCs (0.91±0.23×103 mm3) than that in PBS group (2.30±0.33×103 mm3), or free TAXOL® treatment group (1.70±0.42×103 mm3) (**p<0.01, ***p<0.001, ANOVA, n=6 mice/group). Representative photos for tumors from each group on day 27 post-implant are shown in top images of FIG. 14B. We evaluated the tumor growth inhibition (TGI) rate of each group (see the details in Method). The tumor growth inhibition rate was doubled when mice were treated with PTCs, as compared to the mice treated with an identical dose of free TAXOL® (60.7±1.0 vs. 26.2±1.2, ***p<0.001, student's t-test, n=6 mice/group). These results suggest that PTC can serve as a therapeutic option for refractory tumor by tumor- and drain lymph node-targeting capability of Prot101. It is noted that the mice treated with PTCs showed no morbidity during the course of treatment (FIG. 14C). Furthermore, we confirmed that nodules were widely located in the lung of PBS and free TAXOL® groups as a symptom of metastasis of 4T1 tumor, whereas the mice treated with PTC had no nodules (bottom images in FIG. 14B).

Encouraged by the therapeutic effects for 4T1 tumor model, these protein-drug conjugates were further investigated for cancer therapy in a B16 (melanoma) or LLC1 (lung cancer)-bearing C57BL/6 mice. Drugs were now injected into mice having a tumor volume of approximately 100 mm3 according to the injection schedule identical to the 4T1 study. In FIGS. 14D-14F, PTC displayed the most potent antitumor effects which is consistent to the result for 4T1-bearing mice; on the final day of these studies, the tumor volume of the mice treated with PTC (0.81±0.30×103 mm3 for B16, 0.80±0.21×103 mm3 for LLC1) was the lowest, compared to that with PBS (2.36±0.61×103 mm3 for B16, 1.68±0.30×103 mm3 for LLC1) and with free TAXOL® (1.29±0.50×103 mm3 for B16, 1.21±0.31×103 mm3 for LLC1) (total dose of TAXOL®=32 μg/kg, *p<0.05, **p<0.01, ***p<0.001, analysis of variance [ANOVA], n=6 mice/group). TGI was 68.6±0.8% vs. 47.3±1.2% for B16 and 55.7±0.6% vs. 29.7±0.8% for LLC1. Importantly, mice treated with PTC did not show weight loss.

Most important, is the fact that the dose of taxol injected here as part of Protin-101 is about 1200 times lower than the free taxol used, indicating the potentially lower toxicity of a payload like taxol, gemcitabine or another chemoterapiotic toxic agents.

The results clearly confirm that PTCs induced potent therapeutic efficacy in a variety of tumor models without distinct side effects.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A chimeric protein construct comprising:
(a) a clathrin protein moiety or a functional derivative thereof; and
(b) a chimeric antigen receptor (CAR) comprising:
(i) an ectodomain comprising at least one antigen binding domain;
(ii) a transmembrane domain; and
(iii) an endodomain comprising an intracellular signaling domain.

2. The chimeric protein construct of claim 1, the CAR linked, conjugated, bound, tethered, or fused to the clathrin protein moiety.

3. The chimeric protein construct of claim 2, wherein the clathrin protein moiety and the CAR comprise a fusion protein.

4. The chimeric protein construct of claim 2, further comprising a protein linker operably linking the clathrin protein moiety and the CAR.

5. The chimeric protein construct of any one of claims 1-4, the clathrin protein moiety comprising a clathrin light chain or a clathrin heavy chain or a modified analog thereof.

6. The chimeric protein construct of claim 5, the clathrin protein moiety comprising a clathrin light chain and a clathrin heavy chain.

7. The chimeric protein construct of claim 6, the clathrin heavy chain at least 95% identical to SEQ ID NO: 1 or to SEQ ID NO: 3.

8. The chimeric protein construct of claim 6, the clathrin light chain at least 95% identical to SEQ ID NO: 2 or to SEQ ID NO: 4.

9. The chimeric protein construct of any one of claims 1-8, wherein the clathrin protein moiety comprises a clathrin cage.

10. The chimeric protein construct of any one of claims 1-9, wherein the clathrin protein moiety comprises a three-dimensional clathrin cage structure comprising an outer surface and an inner cavity, the CAR conjugated, bound, linked, tethered, or fused to the outer surface of the three-dimensional clathrin cage structure.

11. The chimeric protein construct of any one of claims 1-10, further comprising a payload.

12. The chimeric protein construct of claim 11, the payload conjugated, bound, linked, tethered, or fused to the clathrin protein moiety or to the CAR.

13. The chimeric protein construct of claim 12, wherein the clathrin protein moiety comprises a three-dimensional clathrin cage structure comprising an outer surface and an inner cavity, the CAR conjugated, bound, linked, tethered, or fused to the outer surface of the three-dimensional clathrin cage structure and the payload conjugated, bound, linked, tethered, fused, or at least partially contained within the inner cavity of the clathrin cage structure.

14. The chimeric protein construct of any one of claims 9-13, the payload comprising a medicament, an imaging agent, or a biomarker.

15. The chimeric protein construct of claim 14, the imaging agent comprising a fluorescence, a radionuclide or an MRI contrast agent.

16. The chimeric protein construct of any one of claims 13-15, the payload comprising a medicament or other pharmaceutical agent for treating or alleviating a disease or an abnormal physiological condition.

17. The chimeric protein construct of claim 16, the disease or abnormal physiological condition comprising a tumor, a cancer, a neurodegenerative disease or condition, an autoimmune disease, a transplant rejection, an inflammatory or neuroinflammatory disease or condition, or an infectious disease.

18. The chimeric protein construct of claim 16, wherein the medicament or other pharmaceutical agent comprises:
(a) gemcitabine or a functional derivative thereof;
(b) paclitaxel or a functional derivative thereof;
(c) docetaxel or a functional derivative thereof;
(d) carboplatin or a functional derivative thereof;
(e) cisplatin or a functional derivative thereof;
(f) azonafide or a functional derivative thereof;
(g) pembrolizumab or a functional derivative thereof;
(h) nivolumab or a functional derivative thereof;
(i) cemiplimab or a functional derivative thereof;
(j) pidilizumab or a functional derivative thereof;
(k) BMS-926559 or a functional derivative thereof;
(l) atezolizumab or a functional derivative thereof;
(m) avelumab or a functional derivative thereof;
(n) durvalumab or a functional derivative thereof;
(o) ipilimumab or a functional derivative thereof;
(p) an anti-programmed cell death protein 1 (anti-PD-1) antibody or antibody derived protein, or an anti-PD-1 antigen-binding domain;
(q) an anti-programmed death-linker 1 (anti-PD-L1) antibody or antibody derived protein, or an anti-PD-L1 antigen-binding domain; or
(r) an anti-cytotoxic T-lymphocyte-associated protein 4 (anti-CTLA4) antibody or antibody derived protein, or an anti-CTLA4 antigen-binding domain; or
(s) any combination thereof.

19. The chimeric protein construct of any one of claims 16-18, the payload comprising a pharmaceutical composition, an antibody, an antibody-drug conjugate, a nucleic acid, a protein, a peptide, or a polypeptide or polynucleotide vector.

20. The chimeric protein construct of claim 19, the payload comprising an antibody-drug conjugate, the antibody or antigen-binding domain thereof recognizing or binding to an antigen on a tumor cell or neoplastic cell, a cancer cell, a cell that promotes growth, mutagenesis, or metastasis of a tumor cell or other neoplastic cell, a neural cell, or an innate immune cell; and the drug thereof comprising a treatment drug.

21. The chimeric protein construct of any one of claims 1-20, the endodomain further comprising:
(a) at least one costimulatory domain;
(b) at least one nuclear factor of activated T cell, B Cell-responsive inducible expression element; or
(c) a combination thereof.

22. The chimeric protein construct of any one of claims 1-21, the ectodomain further comprising a transport signal peptide.

23. The chimeric protein construct of any one of claims 1-22, the CAR further comprising at least one additional intracellular signaling domain.

24. The chimeric protein construct of any one of claims 1-23, the at least one antigen-binding domain of the CAR recognizing or binding to an antigen specific to a target cell of interest.

25. The chimeric protein construct of claim 24, the target cell of interest comprising:
(a) a T-cell;
(b) a B-cell;
(c) a regulatory T (Treg) cell;
(d) a mutant cell;
(e) a diseased cell;
(f) a tumor cell or neoplastic cell;
(g) a cancer cell;
(h) a cell that promotes growth, mutagenesis, or metastasis of a tumor cell or other neoplastic cell;
(i) a cell expressing a specific marker of interest;
(j) a regulatory cell;
(k) an innate immune cell;
(l) a neural cell;
(m) a glial cell;
(n) a secretory cell;
(o) a cell that inhibits or promotes cell death;
(p) an immune effector cell;
(q) a cell that regulates an immune effector cell;
(r) a cell belonging to an infectious agent; or
(s) an immunosuppressive cell.

26. The chimeric protein construct of claim 25, the at least one antigen-binding domain of the CAR recognizing or binding to an antigen on an immune effector cell, a lymphoid cell, a cell that regulates an immune effector cell, a regulatory cell, a lymphoid cell, a secretory cell, or a cell that inhibits or promotes cell death.

27. The chimeric protein construct of claim 26, wherein the target cell of interest is an immune effector cell selected from the group consisting of a T-cell, a regulatory T (Treg) cell, a B-cell, a dendritic cell, or a natural killer (NK) cell.

28. The chimeric protein construct of claim 27, wherein the target cell of interest comprises a breast tumor or cancer cell, a pancreatic ductal adenocarcinoma (PDA) cell, a lymphocyte, a gamma-delta T-cell (γδ-T-cell), a lymph node venule cell, a renal tumor or carcinoma cell, a skin tumor, a melanoma cell, a bladder tumor or cancer cell, a gastric tumor or cancer cell, a lung tumor, a non-small cell lung cancer cell, a lymphoma cell, a mesothelioma cell, a urothelial carcinoma cell, a Merkel-cell carcinoma cell, a head or neck cancer cell, a squamous cell carcinoma cell, a Treg cell, a neural cell, an innate immune cell, an inflammatory cell, or a disease modulating cell.

29. The chimeric protein construct of claim 27 or claim 28, wherein the antigen comprises a proinflammatory or anti-inflammatory cytokine.

30. The chimeric protein construct of claim 29, wherein the antigen comprises interleukin-10 (IL-10), interleukin-2 (IL-2), interleukin-6 (IL-6) or another interleukin; transforming growth factor beta (TGF-β); programmed cell death protein 1 (PD-1); programmed death-ligand 1 (PD-L1); or cytotoxic T lymphocyte-associated protein 4 (CTLA-4).

31. The chimeric protein construct of claim 24, wherein the target cell of interest comprises a lymphoid cell.

32. The chimeric protein construct of claim 24, wherein the antigen comprises L-selectin.

33. The chimeric protein construct of claim 24, wherein the antigen-binding domain of the CAR comprises an anti-peripheral lymph node addressin (PNAd) binding domain.

34. The chimeric protein construct of claim 24, the antibody or antigen binding domain thereof recognizing or binding to an antigen on a tumor cell or neoplastic cell, a cancer cell, or a cell that promotes growth, mutagenesis, or metastasis of a tumor cell or other neoplastic cell.

35. The chimeric protein construct of any one of claims 1-34, the ectodomain of the CAR comprising two antigen-binding domains, each recognizing or binding to a different antigen.

36. The chimeric protein construct of claim 35, the ectodomain of the CAR comprising two antigen-binding domains, wherein a first antigen-binding domain recognizes or binds to an antigen on a first target cell of interest and a second antigen-binding domain recognizes or binds to an antigen on a second target cell of interest.

37. The chimeric protein construct of claim 36, the first target cell of interest comprising an immune effector cell, a cell that regulates an immune effector cell, a regulatory cell, a lymphoid cell, secretory cell, or a cell that inhibits or promotes cell death.

38. The chimeric protein construct of claim 37, wherein the second target cell of interest comprises a tumor cell or neoplastic cell, a cancer cell, or a cell that promotes growth, mutagenesis, or metastasis of a tumor cell or other neoplastic cell, a mutant cell, a diseased cell, or a cell belonging to an inflammatory or infectious agent.

39. The chimeric protein construct of claim 38, wherein the second target cell of interest comprises a lymphoid cell.

40. The chimeric protein construct of any one of claims 1-39, wherein the antigen binding domain comprises an antigen-binding single-chain Fv (scFv).

41. The chimeric protein construct of any one of claims 1-40, wherein the endodomain comprises CD28 or a costimulatory domain thereof, CD137 (4-1BB) or a costimulatory domain thereof, or CD134 (OX40) or a costimulatory domain thereof.

42. The chimeric protein construct of any one of claims 1-41, further comprising an antibody or an antigen-binding fragment thereof, the antibody or antigen-binding fragment thereof comprising an antigen-binding domain distinct from the antigen-binding domain of the CAR and recognizing or binding to an antigen of interest.

43. The chimeric protein construct of claim 42, the antibody or antigen-binding fragment thereof recognizing or binding to a secretory protein or to a cell surface protein.

44. The chimeric protein construct of claim 43, the antibody comprising an antibody-drug conjugate comprising an antibody or an antigen binding domain thereof linked to a drug.

45. The chimeric protein construct of claim 44, the antibody or antigen-binding domain thereof recognizing or binding to an antigen on a tumor cell or neoplastic cell, a cancer cell, or a cell that promotes growth, mutagenesis, or metastasis of a tumor cell or other neoplastic cell; and the drug comprising a cytotoxic drug.

46. A method of producing a chimeric antigen receptor (CAR)-engineered cell of interest in vivo, the method comprising:
(a) providing a chimeric protein construct comprising:
(i) a clathrin protein moiety or a functional derivative thereof; and
(ii) a chimeric antigen receptor (CAR) comprising:
(a) an ectodomain comprising at least one antigen binding domain;
(b) a transmembrane domain; and
(c) an endodomain comprising an intracellular signaling domain;
(b) administering the chimeric protein construct to a subject in need thereof; and
(c) transducing a target cell of interest in the subject with the chimeric protein construct to produce a CAR-engineered cell of interest.

47. The method of claim 46, wherein the step of transducing the target cell of interest with the chimeric protein construct comprises clathrin-mediated endocytosis of the CAR or the chimeric protein construct.

48. The method of claim 46 or claim 47, wherein the target cell of interest is an immune effector cell selected from the group consisting of a T-cell, a regulatory T (Treg) cell, a B-cell, a dendritic cell, or a natural killer (NK) cell.

49. The method of claim 48, wherein the target cell of interest is an immune modulating cell.

50. The method of claim 46 or claim 47, wherein the target cell of interest is a tumor cell or neoplastic cell; a cancer cell; or a cell that promotes growth, mutagenesis, or metastasis of a tumor cell, a neoplastic cell, or a cancer cell; or a pro-inflammatory cell, an anti-inflammatory cell, or an infectious cell.

51. The method of claim 50, wherein the target cell of interest comprises a pancreatic ductal adenocarcinoma (PDA) cell, a lymphocyte, a gamma-delta T-cell (γδ-T-cell), a lymph node venule cell, a breast tumor or cancer cell, a renal tumor or carcinoma cell, a skin tumor, a melanoma cell, a bladder tumor or cancer cell, a gastric tumor or cancer cell, a lung tumor, a non-small cell lung cancer cell, a lymphoma cell, a mesothelioma cell, a urothelial carcinoma cell, a Merkel-cell carcinoma cell, a head or neck cancer cell, a squamous cell carcinoma cell, a Treg cell, a neural cell, an innate immune cell, an inflammatory cell, or a disease modulating cell.

52. The method of claim 46, the CAR linked to the clathrin protein moiety.

53. The method of claim 46, the CAR conjugated, bound, tethered, or fused to the clathrin protein moiety.

54. The method of claim 46, wherein the clathrin protein moiety and the CAR comprise a fusion protein.

55. The method of any one of claims 46-53, the clathrin protein moiety comprising a clathrin light chain, a clathrin heavy chain, or a combination thereof.

56. The method of claim 55, the clathrin heavy chain at least 95% identical to SEQ ID NO: 1 or to SEQ ID NO: 3.

57. The method of claim 55, the clathrin light chain at least 95% identical to SEQ ID NO: 2 or to SEQ ID NO: 4.

58. The method of claim 55, wherein the clathrin protein moiety comprises a clathrin cage.

59. The method of claim 55, the clathrin protein moiety further comprising a payload.

60. The method of claim 59, the payload conjugated, bound, linked, tethered, or fused to the clathrin protein moiety or to the CAR.

61. The method of claim 59, wherein the clathrin protein moiety comprises a three-dimensional clathrin cage structure comprising an outer surface and an inner cavity, the CAR conjugated, bound, linked, tethered, or fused to the outer surface of the three-dimensional clathrin cage structure and the payload conjugated, bound, linked, tethered, fused, or at least partially contained within the inner cavity of the clathrin cage structure.

62. The method of any one of claims 59-61, the payload comprising an imaging agent, a biomarker, or a medicament or other pharmaceutical agent.

63. The method of any one of claims 46-62, wherein the antigen binding domain comprises an antigen-binding single-chain Fv (scFv).

64. The method of claim 63, wherein the antigen-binding scFv comprises a heavy chain and a light chain operably linked by an scFv linker.

65. A method of inhibiting the growth, mutagenesis, or metastasis of a cancer, a tumor, or other neoplasm in a subject in need thereof, the method comprising:
(a) obtaining an antigenic profile of the cancer, the tumor, or the other neoplasm;
(b) providing an antigen binding domain in response to the antigenic profile of the cancer, the tumor, or the other neoplasm, wherein the antigen binding domain binds an antigen specific to the tumor or other neoplasm or an antigen specific to a target cell of interest, wherein:
(i) the target cell of interest promotes or prevents growth, mutagenesis, or metastasis of the tumor or other neoplasm;
(ii) the target cell of interest is a tumor cell, a cancer cell, or another neoplastic cell; or
(iii) the target cell of interest is an immune effector cell selected from the group consisting of a T-cell, a B-cell, a dendritic cell, or a natural killer (NK) cell;
(c) providing a chimeric protein construct, the chimeric protein construct comprising:
(i) a clathrin protein moiety; and
(ii) a chimeric antigen receptor (CAR) comprising:
(a) an ectodomain comprising the antigen binding domain;
(b) a transmembrane domain; and
(c) an endodomain comprising an intracellular signaling domain;
(d) administering the chimeric protein construct to the subject; and
(e) inhibiting the growth, mutagenesis, or metastasis of the tumor or other neoplasm or reducing the size or amount of the tumor or other neoplasm.

66. A method of obtaining an image of a target cell of interest in a subject, the method comprising:
(a) obtaining an antigenic profile of the target cell of interest;
(b) providing an antigen binding domain in response to the antigenic profile of the target cell of interest, wherein the antigen binding domain binds an antigen specific to the target cell of interest, wherein;
(c) providing a chimeric protein construct, the chimeric protein construct comprising:
(i) a clathrin protein moiety;
(ii) a chimeric antigen receptor (CAR) comprising:
(a) an ectodomain comprising the antigen binding domain;
(b) a transmembrane domain; and
(c) an endodomain comprising an intracellular signaling domain; and
(iii) an imaging agent;
(d) administering the chimeric protein construct in a detectable amount to the subject;
(e) incubating the chimeric protein construct in the subject;
(f) detecting the imaging agent; and
(g) generating an image of the target cell of interest.
